(19) United States
(12) Patent Application Publication
(10) Pub. No.: US 2026/0165956 A1
(43) Pub. Date: Jun. 18, 2026

(54) IN VIVO TARGETING OF THERAPEUTIC MOLECULES TO THE RETINA VIA THE OPTICAL SYSTEM OF THE EYE

(71) Applicants: SEMMELWEIS EGYETEM, Budapest (HU); EXPERIMENTICA HOLDING OY, Kuopio (FI); VICHEM CHEMIE KUTATÓ KFT., Budapest (HU)

(72) Inventors: Krisztián András KOVÁCS, Remeteszolos (HU); Giedrius KALESNYKAS, Lempaala (FI); Bálint SZOKOL, Budapest (HU); Eszter ILLYÉS, Budapest (HU); István SZABADKAI, Budapest (HU)

(21) Appl. No.: 19/127,226

(22) PCT Filed: Oct. 16, 2023
(86) PCT No.: PCT/HU2023/050073

§ 371 Date: May 5, 2025

(30) Foreign Application Priority Data
Nov. 4, 2022 (HU) P2200436

(51) Int. Cl. A61K 9/00

(52) U.S. Cl. CPC A61K 9/0053; A61K 9/0048; A61K 2300/00

(57) ABSTRACT

In the present invention azidation is used to render a molecule photoactivable and to influence its binding to its cognate specific binding partner in the eye. Thus, the invention relates to a compound for use in a method of treating a subject with an ocular disease said compound comprising—a conjugated electron system—an active agent moiety, —an azide (N3) moiety comprising an azido group, wherein the 7t electrons of the azido group extend the conjugated electron system to form an extended conjugated electron system, whereby the active agent moiety can be bound to the binding site of the biological target, and the azide moiety can be photoactivated by the light entering via the optical system of the eye, whereby the engineered molecule becomes linked to the biological target via a covalent bond, whereby the compound modulates the said biological target in the eye to provide an improved treatment for said subject.

### FIELD OF THE INVENTION

In the present invention azidation is used to render a molecule photoactivable and to influence its binding to its cognate specific binding partner in the eye. The ambient light is focused onto the said ocular tissue by the optical system of the eye itself, without using an artificial photoactivation step by medical personnel. The photoactivation of the said molecule results in the formation of a covalent chemical bond between the said molecule and its said cognate specific binding partner, that leads to:
(1) the targeting of the said molecule to the said ocular tissue,
(2) the enrichment of the said molecule in the said ocular tissue,
(3) modification of the biological effect of the said molecule as compared to its non-azidated counterpart (hereinafter referred to as “parental molecule”) in a way that is beneficial for the subject to be treated.

### BACKGROUND ART

Gaining access to the retina for the purpose of drug delivery is extremely challenging given the known and well described ocular anatomical barriers [Yamada et al., 2016]. Any method for local delivery would offer the advantage of using extremely small doses of medication and minimizing the potential systemic toxicity in addition to providing a route of delivery. The corneal and conjunctival epithelium, furthermore, the diffusion across the cornea and scleral stroma constitute an important barrier for any molecule applied as eyedrops [Grass et al., 1988], while the blood retinal barrier strictly regulates the transport between the plasma circulating in the retinal capillaries and extracellular space of the neuroretina [Diaz-Coránguez, 2017] thereby restricting the access of drug molecules present in the circulation (such as molecules taken per os and absorbed by the gut).

However, if a method for local delivery is achieved for a given condition, the therapy will likely require extremely small doses relative to the amount required to achieve similar tissue levels using systemic therapy. Local therapy minimizes systemic drug levels and thus limits potential systemic toxicity in addition to providing a route for the therapeutic compounds to reach the retinal tissue [Yamada et al., 2016].

Therefore, several methods have been elaborated for local administration of compounds to the retina, the most important ones include the suprachoroideal, subretinal and the intravitreal delivery route. The suprachoroideal administration can target specific chorioretinal tissues, and provides circumferential spread of the administered molecules that can reach the posterior segment of the eye without affecting the untargeted anterior segment [Wan et al., 2021]. The subretinal injections are most often used for the delivery of viral vectors to locally express therapeutic transgenes [Peng et al., 2017]. Finally, the intravitreal injections are very frequently used to deliver drug molecules into the vitreus [Fagan et al. 2013] and according to the estimations 24.4 million of such injections were performed globally in 2019 [Wan et al., 2021].

Photodynamic therapy (PDT) uses a photosensitizer compound for destruction of an undesired tissue like tumor tissue or blood vessels arising via neovascularization, by introducing said compound to a target tissue of a patient followed by activation of the photosensitizer by low energy directed light. Photoreactive chemicals are typically injected into the patient and irradiated with laser light as they pass through the neovascular elements. This light should be strong enough to activate the chemicals, causing them to emit free radicals that destroy the undesired tissue e.g. blood vessels formed via neovascularization, but should not be strong enough to cause damage to the overlying retina. Clearly this constitutes a danger for healthy tissues and the laser should carefully be focused onto the undesired tissue. Therefore, laser irradiation when used as a part of PDT requires a trained doctor [Wormald 2005]. Of note, PDT never relies on a specific interaction between a drug molecule reaching the eye and its specific target molecule present in the eye. Similarly, PDT never relies on the formation of a covalent bond between a drug molecule photoactivated within the eye and its specific target molecule present in the eye.

In the last decade, the therapeutic decrease of the vascular endothelial growth factor (VEGF) signaling using intravitreally injected molecules became the gold standard to suppress ocular neovascularization. This approach is currently being used almost exclusively to treat, among others, age-related macular degeneration (AMD), proliferative diabetic retinopathy (PDR), diabetic macular oedema (DME), and has almost completely replaced the more invasive PDT that required the intravenous injection of a photosenzitizer substance and the subsequent laser irradiation of the eye to damage and eliminate the undesired blood vessels formed by neovascularization. Nonetheless, in addition to psychological and medical burden that intravitreal injections still place on the patient and thereby decrease the patient compliance, the risks of endophthalmitis [Seong et al., 2022] and submacular haemorrhages [Khoo et al., 2022] associated to intravitreal injections remain unacceptably high which necessitates the elaboration of novel ocular administration routes.

The present inventors have found a new, unexpected way of retinal targeting which also eliminates the need for intravitreal injections. As an exemplary application, a tailored modification of a small molecule VEGFR2 inhibitors such as sunitinib have been carried out to obtain their azido-derivatives.

Fahrenholtz et al. have suggested that an azidated vazopressine peptide hormone analogue of eight amino acids, after photolysis, binds covalently to hormonal receptors in toad bladder and forms an active hormone-receptor complex and proposed the use of this analog for studies of hydroosmotic receptor function and for receptor isolation [Fahrenholtz et al., (1983)].

However, as a light source activating a molecule carrying an azido group, the light naturally projected onto the retina by the optical system of the eye has never been proposed. WO2011/084571 [Rajagopalan, R. 2011] provides preparations and formulations comprising azide derivatives as Type I optical (phototherapeutic) agents in in vivo or ex vivo biomedical procedures, wherein selective tissue injury can be induced with light when the azide-photosensitizers bind to the target tissues, either directly or through attachment to a bioactive carrier or targeting moiety. Preferably the compounds have a photolabile azido group capable of undergoing photoactivation-mediated bond dissociation and/or nitrogen extrusion processes to produce reactive species, that achieve a desired therapeutic effect, such as selective and/or localized tissue damage and/or cell death. The optical agents typically include compositions having a substituted phenyl group with a combination of electron donating and electron withdrawing groups as ring substituents.

Importantly, WO2011/084571 uses the principle of photodynamic therapy. Correspondingly (i) it does not rely on the formation of a covalent bond between the azidated molecule and the target molecule (ii) it does not rely on a specific binding between the azidated molecule and the target molecule before covalent bond formation triggered by photoactivation (iii) it does not rely on the light naturally arriving into the eye, instead, it uses artificial irradiation (iv) it relies on the catalytic production of reactive species (such as radicals) to destroy the nearby tissue in a random manner.

The present inventors have recognized that rendering a molecule photoactivable by azidation allows its covalent binding to its cognate specific binding partner (preferably receptor or enzyme) in any ocular tissue (including but not limited to the retina), using the ambient light that is focused onto the said ocular tissue by the optical system of the eye itself.

As a particular example, a high number of inhibitors of VEGF signaling, in particular VEGFR2 inhibitor, is known in the art [Peng, Fan-Wei et al., 2017, Farghaly, T A et al. 2021, Khanwelkar, R. R, 0.2010, Yang T-H et al. 2017A and 2017B]. In an embodiment the present invention aims at converting such inhibitors by azidation to a compound which is still capable of inhibiting VEGF signaling and useful in the present invention. The compounds of the present invention are designed in such a way that they have a cognate binding partner in the eye (such as a receptor or enzyme), e.g. a biological target, e.g. a receptor in the VEGF signaling pathway, in particular VEGFR2. Therefore, the compounds of the present invention can be administered by routes other than injection, e.g. orally, the active agent moieties specifically bind to their cognate binding partner (e.g. a receptor) in the eye whereas covalent binding is reached due to activation of the azido group by natural light seen by the patient or animal to be treated. Thereby the active agents accumulate in the eye even when a low dose of the compounds is administered and a low concentration is maintained in the circulation by a correctly designed regimen of dosing, taking into consideration the pharmacokinetic properties of the azidated compound.

### BRIEF DESCRIPTION OF THE INVENTION

In the present invention azidation is used to render a molecule photoactivable and to influence its binding to its cognate specific binding partner (preferably receptor or enzyme) in any ocular tissue which is exposed to ambient light (including but not limited to the retina), using the ambient light that is focused onto the said ocular tissue by the optical system of the eye itself, without using an artificial photoactivation step by medical personnel (FIG. 1.). The photoactivation of the said molecule results in the formation of a covalent chemical bond between the said molecule and its said cognate specific binding partner, that leads to:
(4) the targeting of the said molecule to the said ocular tissue,
(5) the enrichment of the said molecule in the said ocular tissue,
(6) modification of the biological effect of the said molecule as compared to its non-azidated counterpart (hereinafter referred to as “parental molecule”) in a way that is beneficial for the subject to be treated.

Ambient light may be provided by natural light or by artificial light of an appropriate wavelength range. The light is never laser light but the light seen by the patient. In a preferred embodiment the ambient light is visible light.

For example, the illumination may be provided by a light source, e.g. lamp in a room where the patient resided for a period of treatment. Or, alternatively, illumination may be provided by sunlight during a period when the patient perambulated an outdoor area.

In an alternative embodiment the ambient light is provided by a wearable device, like an “eye-glass”, which mildly illuminates the eye i.e. the ocular tissues (such as the retina) the light is naturally projected onto.
1. Thus, the invention relates to a compound for use in a method of treating a subject with an ocular disease said compound comprising
   a conjugated electron system
   an active agent moiety, said moiety being a modulating entity (preferably a ligand or a substrate, including a functional analogue of a natural ligand or a functional analogue of a natural substrate) and thereby being useful for treatment of said ocular disease,
   an azide (N₃) moiety comprising an azido group,
   wherein the π electrons of the azido group extend the conjugated electron system to form an extended conjugated electron system,
   whereby the active agent moiety can be bound to the binding site of the biological target, and the azide moiety can be photoactivated and linked to the biological target via a covalent bond,
   whereby the compound modulates the said biological target in the eye to provide treatment for said subject, in particular an improved treatment for said subject.

Thus, the invention also relates to a compound for use in a method of treating a subject with an ocular disease said compound comprising
an active agent moiety, said moiety being a modulating entity (preferably a ligand or a substrate, including functional analogues of a natural ligand or functional analogues of a natural substrate) of a biological target (preferably a receptor or an enzyme), preferably exerting full or partial activating or inhibiting effect, and thereby being useful for treatment of said ocular disease,
a conjugated moiety having a conjugated electron system,
an azide (N₃) moiety comprising an azido group,
wherein the π electrons of the azido group form a conjugated electron system together with the conjugated electron system of the conjugated moiety,
whereby the active agent moiety can be bound to the binding site of the biological target, the azide moiety can be photoactivated and linked to the biological target via a covalent bond,
whereby the compound modulates the said biological target in the eye to provide treatment for said subject.

In a preferred embodiment the conjugated moiety is part of the active agent moiety.

In a preferred embodiment the conjugated moiety is a moiety different from the active agent moiety. In a preferred embodiment the conjugated moiety is a linker moiety having a conjugated electron system.

In an embodiment the conjugated moiety and the active agent moiety overlap, optionally identical or optionally forming part of each other.

In an embodiment the linker moiety provides flexibility to the azide moiety to bind to an appropriate amino acid of the biological target.

Preferably the conjugated π (pi) electron system of the compound comprises at least 3, more preferably at least 4, even more preferably at least 5 non-sigma electron pairs excluding the π (pi) electron system of the azide moiety.

Preferably the extended conjugated π (pi) electron system of the compound comprises at least 3, more preferably at least 4, even more preferably at least 5 non-sigma electron pairs plus the π (pi) electron system of the azide moiety.

Preferably, due to the conjugated system, the azide moiety is excitable with a light of a wavelength of at least 350 nm, preferably at least 400 nm, and up to 800 nm, preferably up to 600 nm. Preferably the light is white light, in particular 400-800 nm, preferably 400-600 nm.

Preferably the azide moiety, upon excitation forms a reactive radical capable of covalently binding to the biological target in the illuminated eye tissue. The activated azide moiety may form, preferably, a nitrene group or a reactive cyclic ketene-imine created via ring-expansion. In a preferred embodiment the conjugated moiety is part of the active agent moiety, e.g. the conjugated moiety is the same as the active agent moiety.

In the present invention the active agent moiety and thus the compound of the invention can be bound to the binding site of the biological target which can be tested by a binding assay. Such binding assays are known to a person skilled in the art. For example, the active agent moiety or the compound of the invention is contacted with the biological target and binding is measured. As an other example, the active agent moiety or the compound of the invention is contacted with the biological target and the activity of the biological target (or a change in activity thereof) is measured. In case the active agent moiety is an inhibitor the compound is an inhibitor of the biological target. For example, the active agent moiety or the compound of the invention is contacted with the biological target and the activity of the biological target (or a reduction of the activity thereof) is measured.

In a further embodiment said compound is an inactive prodrug, comprising a metabolizable group linked to the molecule that is preferentially cleavable by intracellular esterases. In an embodiment the metabolizable group may be linked to a ring nitrogen atom.
2. In a preferred embodiment said azidated compound is administered orally and/or formulated for oral administration and is delivered into the eye wherein its azido group is converted to a reactive radical upon exposure to ambient light. In a preferred embodiment light protection is provided by a protective coating e.g. a capsule wall.

Preferably the azidated compound is formulated to allow transfer of the compound through the blood-retina barrier. In an embodiment, the azidated compound comprises a moiety that permits the active transporter (such as vitamin transporter) mediated uptake into the desired part of the retinal tissue or into the desired cell. Preferably the azidated compound is a ligand or an enzyme substrate and therefore in the eye (once transferred through the blood-retina barrier) is targeted to its receptor or enzyme.
3. Preferably said compound for use is an aryl-azide compound, wherein the azido group forms a reactive radical upon illumination by light, preferentially a nitrene radical or a reactive cyclic ketene-imine in the eye via contacting ambient light, e.g. light naturally entering into the eye. The π electrons of the azido group extend the conjugated electron system to form an extended conjugated electron system.
4. Preferably said compound for use according to any of paragraphs 1 to 3 in treating the said subject suffering from an ocular disease that involves a targetable endogenous biomolecule (such as a receptor or an enzyme). Preferably in the ocular disease a targetable endogenous biomolecule is part of the pathomechanism. The group of the said ocular diseases includes but is not limited to ocular neovascularization.

Preferably the said ocular disease is selected from the group of ocular diseases defined in paragraph 9 and preferred groups defined therein.
5. Preferably the compound for use according to any of paragraphs 1 to 4 (and thus the active agent moiety) is a VEGF or PDGF signaling inhibitor, preferably a VEGF receptor (VEGFR) inhibitor (in particular a VEGFR inhibitor selected from the group consisting of VEGFR1, VEGFR2 and VEGFR3 inhibitors), more preferably a VEGFR2 inhibitor.

In this preferred embodiment of the invention the active agent moiety and thus the compound of the invention can be bound to a VEGFR inhibitor selected from the group consisting of VEGFR1, VEGFR2 and VEGFR3 inhibitors, preferably to VEGFR2 which can be tested by a VEGFR2 binding assay or a VEGFR2 signaling assay. In the preferred embodiment described in this paragraph, the second option is preferred given that it yields functional results. Such an assay is known to a person skilled in the art and, as is described in the present document as tool to demonstrate the efficiency of our exemplary compounds.

Briefly, the compound is applied in an assay where VEGFR2-dependent signaling can be measured and the effect of the light is quantified in the said assay.
6. Preferably the compound for use according to any of paragraphs 1 to 5, in particular paragraph 5, is an indole derivative comprising an indole moiety, even more preferably it comprises an indole-2-one moiety (hereinafter also referred to as an “oxindole” moiety) wherein the benzene ring of the oxindole is substituted with an azido group. Such preferred compound can be considered (and will be hereinafter referred to) as an oxindole derivative.
7. In a preferred embodiment the oxindole derivative is an oxindole derivative VEGFR-inhibitor, preferably an oxindole derivative with stronger inhibitory potential against VEGFR2 than against other VEGFR proteins (such as VEGFR1 or VEGFR3), preferably a compound for use according to any of paragraphs 1 to 6, in particular paragraph 6.
8. In a preferred embodiment the invention relates to a compound for use according to any of paragraphs 1 to 7, in particular paragraph 6 or 7, wherein preferably the oxindole derivative VEGFR2 inhibitor has a general formula (X) or optionally (X.1)

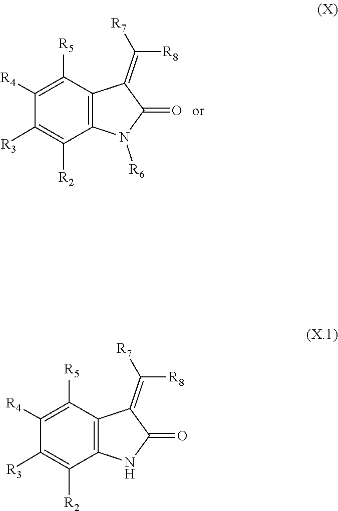

wherein in the formula
at least one of R₂, R₃, R₄ and R₅ is an azido group (N₃);
wherein any one of R₂, R₃, R₄ and R₅ which is different from an azido group, is selected from the group consisting of
   H, Me, halogenide, pseudohalogenide k), —OH, —SH, —OMe, —OEt, —NO₂, —NH₂, —NHMe, —COOH, CONH₂—CF₃; preferably H, halogenide, pseudohalogenide —O, —SH, in particular H or halogenide, more particularly halogenide,
   substituted or unsubstituted C1-C8 alkyl, C2-C8 alkenyl, C2-C8 alkynyl, C1-C8 alkoxy, C1-C8 alkenyloxy, C1-C8 alkynyloxy, C1-C8 alkylamide, C1-C8 alkenylamide, C1-C8 alkynylamide, (wherein optionally C8 alkynylxy, said alkynyloxy, C1-C8 alkenylamide C1-C8 alkynylamide are left out) C6-C10 aryl, C7-C12 alkylaryl (aralkyl), 5 to 10 membered heteroaryl, 6-12 membered alkyl-heteroaryl, C1-C5 amide a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, if any, being selected from halogenide, pseudohalogenide, —OH, —SH, —OMe, —NO₂, —NH₂, —NHMe,
      NR₂₁R₂₂, wherein R₂₁ and R₂₂ are, independently selected from H, methanesulfonyl, ethanesulfonyl, phenylsulfonyl, substituted or unsubstituted C1-C8 alkyl and C1-C8 alkoxy, said substituent, if any, preferably being selected from halogenide, pseudohalogenide, —OH, —SH, —OMe, —NO₂, —NH₂, —NHMe, more preferably halogenide, wherein preferably at least one of R₂₁ and R₂₂ is H, Me or Et,
      SO₂NR₂₃R₂₄, wherein R₂₃ and R₂₄ are, independently selected from H, substituted or unsubstituted C1-C8 alkyl, preferably C1-C4 alkyl, C6-C10 aryl, C7-C12 alkylaryl (aralkyl), 5 to 10 membered heteroaryl, 6-12 membered alkyl-heteroaryl, said substituent, if any, preferably being selected from halogenide, pseudohalogenide, —OH, —SH, —OMe, —NO₂, —NH₂, —NHMe, more preferably halogenide, wherein preferably at least one of R₂₃ and R₂₄ is H, Me or Et
   ureido, preferably aryl-ureido or heteroaryl-ureido, preferably C1-C20 aryl-ureido, more preferably a phenyl-ureido optionally substituted with (preferably in para position) C1-C4 alkyl, C2-C4 alkenyl, C2-C4 alkynyl, C1-C4 alkoxy, C1-C4 carbonyl (preferably C2-C4 alkylcarbonyl, C3-C4 alkenylcarbony or C3-C4 alkynylcarbonyl), C1-C4 alkylamide, C6-C10 aryl, C7-C12 alkylaryl (aralkyl), 5 to 10 membered heteroaryl, 6-12 membered alkyl-heteroaryl, C1-C5 amide, C1-C6 carboxyl (preferably carboxyl, C2-C6 alkylcarboxyl, a C3-C6 alkenylcarboxyl, a C3-C6 alkynylcarboxyl), C2-C6 carboxylate ester, halogenide, pseudohalogenide, —OH, —SH, —OMe, —NO₂, —NH₂, —NHMe, highly preferably (para-metoxy-phenyl)-ureido,
   R₆ is selected from H and an in vivo metabolizable (preferably an intracellularly metabolizable) moiety whereby the compound is a prodrug; and/or a moiety selected from the group consisting of the following moieties:
      a substituted or unsubstituted C1-C4 alkyloxy group linked via a carbon to the nitrogene atom of the oxindole structure (in particular a C1-alcoxy, preferably a CH₂—O— moiety) preferably acylated to be an ester by an —C(O)—R₃₁ group, wherein R₃₁ is selected from the group consisting of OR₃₂, SR₃₂, and N(R₃₂)₂; and
         R₃₁ or R₃₂ is selected from the group consisting of
         H, unsubstituted or substituted C1-C30 alkyl, preferably a C1-C12 alkyl, more preferably a C1-C8 or a C1-C6 alkyl, in particular a C1-C4 alkyl, C2-C30 alkenyl preferably a C1-C12 alkenyl, more preferably a C1-C8 or a C1-C4 alkenyl, in particular a C1-C6 alkenyl, C2-C30 alkynyl, preferably a C1-C12 alkynyl, more preferably a C1-C8 or a C1-C6 alkynyl, in particular a C1-C4 alkynyl; C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group; or wherein the said alkyl, alkenyl or alkyl group has a substitutent (is substituted) by said cycloalkyl, aryl, heterocyclyl, heteroaryl, hydroxylalkyl, carboxylalkyl or alkylamido group,
         said substituent of the C1-C4 alkyloxy group if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe,
         wherein it is noted that esters can typically be cleaved by intracellular esterases that are not present in the extracellular space,
   or R₆ is a or a C(O)—R₃₃ group forming an amide bond with the ring N, wherein R₃₃ group is selected from C1-C30 alkyl, preferably a C1-C12 alkyl, more preferably a C1-C8 or a C1-C6 alkyl, in particular a C1-C4 alkyl, C2-C30 alkenyl, preferably a C1-C12 alkenyl, more preferably a C1-C8 or a C1-C6 alkenyl, in particular a C1-C4 alkenyl, C2-C30 alkynyl, preferably a C1-C12 alkynyl, more preferably a C1-C8 or a C1-C6 alkynyl, in particular a C1-C4 alkynyl; C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group; or wherein the said alkyl, alkenyl or alkyl group has a substitutent (is substituted) by said cycloalkyl, aryl, heterocyclyl, heteroaryl, hydroxylalkyl, carboxylalkyl or alkylamido group, wherein in a particular embodiment said R₃₃ group is selected from a C1-C8 alkyl, in particular a C1-C4 alkyl, wherein the said alkyl, has a substituent (is substituted by a group selected from) a 4-15 membered heterocyclyl,
   in a particularly preferred embodiment R₆ comprises a biotinyl group with or without a linker,
   or in an alternative embodiment R₆ is selected from a substituted or unsubstituted C1-C4 alkyl, C2-C4 alkenyl, C2-C4 alkynyl, C2-C8 alkylester, C3-C8 alkenylester or C3-C8 alkynylester, said substituent, if any, being selected from halogenide, pseudohalogenide, —OH, —SH, —OMe, —NO₂, —NH₂, —NHMe,
   or R₆ is a substituted or unsubstituted C1-C4 alkyloxy group linked via the alkyloxy oxygen to the ring N forming an N—O bond, said substituent on the C1-C4 alkyloxy group (in particular a C1-alcoxy, preferably a CH₂—O— moiety), if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe,
   R₇ and R₈ are selected from the group consisting of a substituted or unsubstituted aryl, in particular a C6-C10 aryl, a heteroaryl, in particular a 5 to 10 membered heteroaryl, a H, amine, preferably a C1-C5 amine, amide, preferably a C1-C5 amide, C2-C6, preferably a C2-C3 alkenyl, a C1-C6 carbonyl, (preferably a C2-C6 alkylcarbonyl, C3-C6 alkenylcarbonyl, C3-C6 alkynylcarbonyl,) a C1-C6 carboxyl, (preferably a C2-C6 alkylcarboxyl, C3-C6 alkenylcarboxyl, C3-C6 alkynylcarboxyl) a C1-C6 carboxylate ester, (preferably a C2-C6 alkylester, C3-C6 alkenylester or C3-C6 alkynylester), wherein if any of R₇ and R₈ is substituted, said substituent, if any, being selected from halogenide, pseudohalogenide, —OH, —SH, —OMe, —NO₂, —NH₂, —NHMe,
with the proviso that at least one of R₇ and R₈ is different from H,
   preferably at least one, preferably one of R₇ and R₈ is selected from the group consisting of a substituted aryl, in particular a C6-C10 aryl, and a substituted heteroaryl, in particular a 5 to 10 membered heteroaryl and
   preferably at least one of R₇ and R₈ is selected from a group having the formula A1 or in the non-N-substituted form A1.1

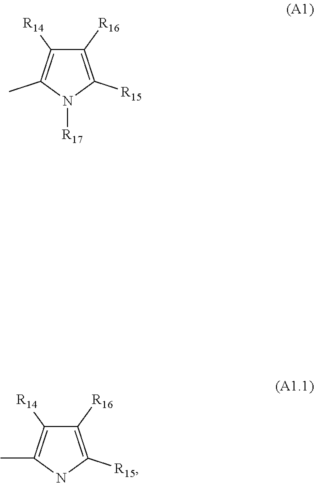

wherein
R₁₄ is selected from H and a C1-C3 alkyl or C2-C3 alkenyl preferably wherein the π electron pair of said C2-C3 alkenyl is conjugated with the π electron system of the pyrrole ring, said C1-C3 alkyl or C2-C3 alkenyl being optionally substituted with a group selected from a halogenide, a C6-C10 aryl or a 5-10 membered heteroaryl, R₁₄ is selected from H and methyl,
   (or in an alternative embodiment R₁₄ is a group as defined for R₁₆, provided that R₁₆ is a group as defined for R₁₄ in the previous paragraph),
R₁₅ is selected from H and a C1-C3 alkyl or C2-C3 alkenyl preferably wherein the π electron pair of said C2-C3 alkenyl is conjugated with the π electron system of the pyrrole ring, said C1-C3 alkyl or C2-C3 alkenyl being optionally substituted with a group selected from a halogenide, a C6-C10 aryl or a 5-10 membered heteroaryl, R₁₅ is selected from H and methyl,
   (or in an alternative embodiment R₁₅ is a group as defined for R₁₆, provided that R₁₆ is a group as defined for R₁₅ in the previous paragraph),
R₁₆ is selected from
H, substituted or unsubstituted C1-C4 alkyl, C2-C4 alkenyl, C2-C4 alkynyl, C1-C4 alkoxy, C1-C6 carbonyl (preferably C2-C6 alkylcarbonyl, C3-C6 alkenylcarbony or C3-C6 alkynylcarbonyl), C1-C6 alkylamide, C6-C10 aryl, C7-C12 alkylaryl (aralkyl), 5 to 10 membered heteroaryl, 6-12 membered alkyl-heteroaryl, C1-C5 amide, C1-C6 carboxyl (preferably carboxyl, C2-C6 alkylcarboxyl, a C3-C6 alkenylcarboxyl, a C3-C6 alkynylcarboxyl) and a C2-C6 carboxylate ester said substituent, if any, being selected from halogenide, pseudohalogenide, —OH, —SH, —OMe, —NO₂, —NH₂, —NHMe, and
a substituted amine or amide wherein preferably said amide is bound via the carbonyl to the pyrrole ring thereby the π electrons of the oxo group forming part of the conjugated system of the pyrrole ring,
said amine or amide substituent in R₁₆ being selected from
substituent 1 (S₁) being a C1-C8 alkyl (preferably C1-C4 or C2-C3 alkyl) preferably substituted with a substituent selected from
   an amine; said amine being optionally substituted with one or two C1-C4 or C2-C3 alkyl or a group as defined as substituent S₃ below, optionally a cyclic polyether forming a tertier amine, (i.e. said amine being a secondary or tertiary amine), and
   a group as defined as substituent S₂ below,
substituent 2 (S₂) being a 5 to 10 membered (preferably 5 to 6 membered) heterocycle, preferably heteroaryl and a C6-C10 aryl, said heterocycle or aryl being optionally substituted with a group having the formula X—R₁₀ wherein X is selected from NH, NR₁₁, R₁₁ being selected from C1-C3 alkyl, C1-C3 alkenyl and C1-C3 alkoxy), O, S, C1-C3 alkyl and C1-C3 alkenyl, and R₁₀ is selected from a 5 to 10 membered heterocycle or a C6-C10 aryl, optionally further substituted with 1 to 4 membered group selected from alkyl, alkenyl, amide, carboxyl alkylcarbonyl, alkoxy and halogenide,
substituent 3 (S₃) being a polyether, preferably a polyethylene glycol, wherein the number ether —O— is 2 to 12, preferably 3 to 9, (or in an alternative embodiment R₁₆ is a group as defined for R₁₅ or a salt or solvate thereof, and
   R₁₇, if present, is selected from H and optionally an in vivo metabolizable group whereby the compound is a prodrug; preferably an intracellularly metabolizable group; preferably R₁₇, once present, is
      a substituted or unsubstituted C1-C4 alkyloxy group linked via a carbon to the nitrogene atom of the oxindole structure (in particular a C1-alcoxy, preferably a CH₂—O— moiety) preferably acylated to be an ester by an —C(O)—R₄₁ group, wherein R₄₁ is selected from the group consisting of OR₄₂, SR₄₂, and N(R₄₂)₂; and
         R₄₁ or R₄₂ is selected from the group consisting of
         H, unsubstituted or substituted C1-C30 alkyl, preferably a C1-C12 alkyl, more preferably a C1-C8 or a C1-C6 alkyl, in particular a C1-C4 alkyl, C2-C30 alkenyl preferably a C1-C12 alkenyl, more preferably a C1-C8 or a C1-C4 alkenyl, in particular a C1-C6 alkenyl, C2-C30 alkynyl, preferably a C1-C12 alkynyl, more preferably a C1-C8 or a C1-C6 alkynyl, in particular a C1-C4 alkynyl; C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group; or wherein the said alkyl, alkenyl or alkyl group has a substitutent (is substituted) by said cycloalkyl, aryl, heterocyclyl, heteroaryl, hydroxylalkyl, carboxylalkyl or alkylamido group,
said substituent of the C1-C4 alkyloxy group if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe,
or R₁₇ is a or a C(O)—R₃₃ group forming an amide bond with the ring N, wherein R₃₃ group is selected from C1-C30 alkyl, preferably a C1-C12 alkyl, more preferably a C1-C8 or a C1-C6 alkyl, in particular a C1-C4 alkyl, C2-C30 alkenyl, preferably a C1-C12 alkenyl, more preferably a C1-C8 or a C1-C6 alkenyl, in particular a C1-C4 alkenyl, C2-C30 alkynyl, preferably a C1-C12 alkynyl, more preferably a C1-C8 or a C1-C6 alkynyl, in particular a C1-C4 alkynyl; C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group; or wherein the said alkyl, alkenyl or alkyl group has a substitutent (is substituted) by said cycloalkyl, aryl, heterocyclyl, heteroaryl, hydroxylalkyl, carboxylalkyl or alkylamido group, wherein in a particular embodiment said R₃₃ group is selected from a C1-C8 alkyl, in particular a C1-C4 alkyl, wherein the said alkyl, has a substituent (is substituted by a group selected from) a 4-15 membered heterocyclyl,
in a particularly preferred embodiment R₁₇ comprises a biotinyl group with or without a linker,
   or in an alternative embodiment R₁₇ is selected from a substituted or unsubstituted C1-C4 alkyl, C2-C4 alkenyl, C2-C4 alkynyl, C2-C8 alkylester, C3-C8 alkenylester or C3-C8 alkynylester, said substituent, if any, being selected from halogenide, pseudohalogenide, —OH, —SH, —OMe, —NO₂, —NH₂, —NHMe,
   or R₁₇ is a substituted or unsubstituted C1-C4 alkyloxy group linked via the alkyloxy oxygen to the ring N forming an N—O bond, said substituent on the C1-C4 alkyloxy group (in particular a C1-alcoxy, preferably a CH₂—O— moiety), if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe.

In a preferred embodiment the compound for use according to the invention, preferably any of paragraphs 1 to 7, in particular paragraph 6 or 7, has formula (X.1) whereas the substitutents, preferably the substitutents R₂, R₃, R₄, R₅, R₇ and R₈, are as defined above or as defined herein:

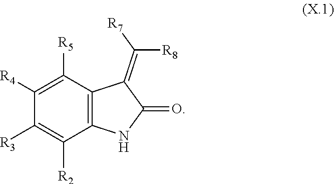

In an alternative wording, R₂, R₃, R₄ and R₅ are selected from the substituents listed above or below in any one of the respective paragraphs and an azide substituent, wherein at least one of R₂, R₃, R₄ and R₅ is an azide. In a preferred embodiment one or two, preferably one of R₂, R₃, R₄ and R₅ is an azide. In a preferred embodiment one, two or three, in particular two or three, more particularly two of R₂, R₃, R₄ and R₅ is H.

In a particular embodiment any one of R₂, R₃, R₄ and R₅ which is different from an azido group, is selected from the group consisting of H, Me, halogenide, pseudohalogenide, —OH, —SH, —OMe, OEt, —NO₂, —NH₂, —NHMe, —COOH, —CONH₂—CF₃; preferably H, halogenide, pseudohalogenide, —OMe, —OH, —SH, in particular H or a halogenide, more particularly a halogenide.

In a preferred embodiment the oxindole derivative of the invention for use as defined herein, preferably the oxindole derivative VEGFR2 inhibitor has a general formula (X.2.1) or its N-substituted prodrug variant (X.2.)

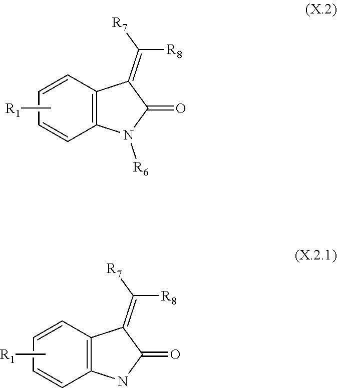

wherein in the formula
R₁ is an azido group and may be connected to any carbon atom of the benzene ring of the indole moiety, preferably to carbon 5 or 6,
R₇ and R₈ are as defined above or herein.

The skilled person will understand that the benzene ring of the oxindole group may be substituted at any other position as taught for any of formulae X1, X.2, I or II, preferably by a single further substitutent, preferably the said single further substituent is a halogenide, preferably in particular Cl or F,
wherein, R₆, if present, is as defined herein, in paragraph 8.
9. The invention relates to a compound for use in treating a subject suffering from an ocular disease that involves a targetable endogenous biomolecule (such as a receptor or an enzyme),
said compound comprising
   a conjugated electron system
   an active agent moiety, said moiety being a modulating entity (preferably a ligand or a substrate, including a functional analogue of a natural ligand or a functional analogue of a natural substrate) and thereby being useful for treatment of said ocular disease,
   an azide (N₃) moiety comprising an azido group,
wherein the π electrons of the azido group extend the conjugated electron system to form an extended conjugated electron system.

In a preferred embodiment the invention relates to a compound for use according to any of paragraphs 1, 2, 3, 4, 5, 6, 7 or 8, in particular any one of paragraphs 5 to 8, for use in treating a subject suffering from an ocular disease that involves a targetable endogenous biomolecule (such as a receptor or an enzyme).

Preferably the subject suffers from ocular neovascularization and/or the mechanism of action of the said compound is based on inhibiting the said ocular neovascularization.

Preferably the pharmaceutical composition is for use in the prevention or reduction of ocular neovascularization in the subject.

Preferably the said ocular disease being selected from the group consisting of
macular degeneration, in particular age-related macular degeneration (AMD),
retinopathies, in particular diabetic retinopathies, proliferative retinopathies, e.g proliferative diabetic retinopathy (PDR),
macular oedema, in particular diabetic macular oedema (DME),
retinal vein occlusion (RVO),
open angle glaucoma (OAG),
angle closure glaucoma (ACG),
congenital glaucoma (CoG).

Preferably said ocular disease being selected from neurodegenerative conditions in glaucoma, preferably neurodegeneration in OAG, ACG or CoG.

Preferably said ocular disease being selected from any other ocular disease where a targetable endogenous biomolecule is part of the pathomechanism.

More preferably the said ocular disease involves neovascularization, preferably neovascularization that can be blocked by the inhibition of ocular VEGF signaling or by the inhibition of the ocular VEGFR2 receptor.
10. In a particularly preferred embodiment the invention relates to a compound having general formula (I.1) or the non-N-substituted variant (I.1.1), preferably a compound for use according to any of paragraphs 1 to 9, in particular paragraphs 1, 2, 3, 4, 5 or 9,

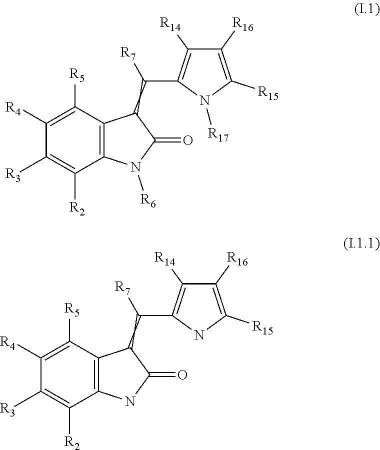

preferably said compound having general formula (I.1) or (I.1.1) has general formula (I.3) or (I.3.1), respectively,

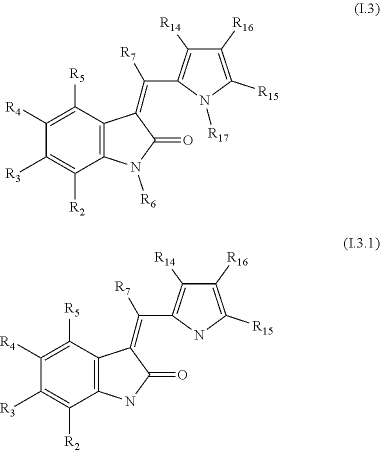

i.e. said compound comprising a “pyrrol-methylidene-oxindole” (3-[(1H-pyrrol-2-yl)methylidene]-1,3-dihydro-2H-indol-2-one) moiety,
wherein in the formula
wherein R₂, R₃, R₄ and R₅ and, if present, R₆, are as defined in paragraph 8 and at least one of R₂, R₃, R₄ and R₅ is azido,
preferably
   at least one of R₂, R₃, R₄ and R₅ is an azido group (N₃);
wherein any one of R₂, R₃, R₄ and R₅ which is different from an azido group, is selected from the group consisting of
   H, Me, halogenide, pseudohalogenide, —OH, —SH, —OMe, —OEt, —NO₂, —NH₂, —NHMe, —COOH, CONH₂—CF₃; preferably H, halogenide, pseudohalogenide, —OMe, —OH, —SH, in particular H or halogenide,
   substituted or unsubstituted C1-C4 alkyl, C1-C4 alkenyl, C1-C4 alkoxy, C1-C4 alkylcarbonyl, C1-C4 alkenylcarbonyl, C6 aryl, C7-C8 alkylaryl (aralkyl), 5 to 6 membered heteroaryl, 6-8 membered alkyl-heteroaryl, said substituent, if any, being selected from halogenide, pseudohalogenide, —OH, —SH, —OMe, —NO₂, —NH₂, —NHMe, preferably —OH, —OMe, —NH₂, —NHMe and halogenide, more preferably halogenide, wherein preferably at least one of R₂₁ and R₂₂ is H, Me or Et, preferably H or Me,
   NR₂₁R₂₂, wherein R₂₁ and R₂₂ are, selected from H, methanesulfonyl, ethanesulfonyl, phenylsulfonyl, substituted or unsubstituted C1-C4 alkyl and C1-C4 alkoxy, said substituent, if any, preferably being selected from halogenide, pseudohalogenide, —OH, —SH, —OMe, —NO₂, —NH₂, —NHMe, preferably —OH, —OMe, —NH₂, —NHMe and halogenide, more preferably halogenide, wherein preferably at least one of R₂₁ and R₂₂ is H, Me or Et, preferably H or Me,
   SO₂NR₂₃R₂₄, wherein R₂₃ and R₂₄ are, independently selected from H, substituted or unsubstituted C1-C4, C6 aryl, C7-C8 alkylaryl (aralkyl), 5 to 6 membered heteroaryl, 6-8 membered alkyl-heteroaryl, said substituent, if any, preferably being selected from halogenide, pseudohalogenide, —OH, —SH, —OMe, —NO₂, —NH₂, —NHMe, preferably —OH, —OMe, —NH₂, —NHMe and halogenide, more preferably halogenide, wherein preferably at least one of R₂₁ and R₂₂ is H, Me or Et, preferably H or Me, wherein preferably at least one of R₂₃ and R₂₄ is H, Me or Et,
   ureido, preferably aryl-ureido or heteroaryl-ureido, preferably phenyl-ureido optionally substituted with (preferably in para position) C1-C3 alkyl, C2-C4 alkenyl, C2-C4 alkynyl, C1-C4 alkoxy, halogenide, pseudohalogenide, —OH, —SH, —OMe, —NO₂, —NH₂, —NHMe, in particular C1-C2 alkoxy, halogenide, methyl or unsubstituted, highly preferably (para-metoxy-phenyl)-ureido,
wherein at least one of R₂, R₃, R₄ and R₅ is azido (preferably one or two, preferably one of R₂, R₃, R₄ and R₅ is azido);
   R₆ is selected from H and a group as described above in paragraph 8; and
   R₃₂ is selected from the group consisting of H, C1-C30 alkyl, C2-C30 alkenyl, C2-C30 alkynyl, C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group,
   said substituent on the C1-C4 alkyloxy group (in particular a C1-alcoxy, preferably a CH₂—O— moiety), if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe,
R₁₄ is selected from H and a C1-C3 alkyl or C2-C3 alkenyl preferably wherein the π electron pair is conjugated with the π electron system of the pyrrole ring, said C1-C3 alkyl or C2-C3 alkenyl being optionally substituted with a group selected from a halogenide, a C6-C10 aryl or a 5-10 membered heteroaryl, R₁₄ is selected from H and methyl,
R₁₅ is selected from H and a C1-C3 alkyl or C2-C3 alkenyl preferably wherein the π electron pair is conjugated with the π electron system of the pyrrole ring, said C1-C3 alkyl or C2-C3 alkenyl being optionally substituted with a group selected from a halogenide, a C6-C10 aryl or a 5-10 membered heteroaryl, R₁₅ is selected from H and methyl,
R₁₆ is selected from
H, substituted or unsubstituted C1-C4 alkyl, C2-C4 alkenyl, C2-C4 alkynyl, C1-C4 alkoxy, —C(O)H, C2-C4 alkylcarbonyl, C3-C4 alkenylcarbonyl, C3-C4 alkynylcarbonyl, C1-C4 alkylamide, C1-C4 amide, carboxyl, C2-C4 alkylcarboxyl, a C3-C4 alkenylcarboxyl, a C3-C4 alkynylcarboxyl, and a C2-C4 carboxylate ester said substituent, if any, being selected from halogenide, pseudohalogenide, —OH, —SH, —OMe, —NO₂, —NH₂, —NHMe, and,
preferably, a substituted amine or amide wherein preferably said amid is bound via the carbonyl to the pyrrole ring thereby the π electrons of the oxo group forming part of the conjugated system,
said amine or amide substituent being selected from
substituent S₁ being a C1-C4 alkyl (preferably C1-C4 or C2-C3 alkyl) preferably substituted with a substituent selected from
   an amine; said amine being optionally substituted with one or two C1-C4 or C2-C3 alkyl or a group as defined as substituent S₃ below, optionally a cyclic polyether forming a tertier amine, (i.e. said amine being a secondary or tertiary amine), and
      a group as defined as substituent S₂ below,
substituent S₂ being a 5 to 10 membered (preferably 5 to 6 membered) heterocycle, preferably heteroaryl and a C6-C10 aryl, said heterocycle or aryl being optionally substituted with a group having the formula X—R₁₀ wherein X is selected from NH, NR₁, R₁₁ being selected from C1-C3 alkyl, C1-C3 alkenyl and C1-C3 alkoxy), O, S, C1-C3 alkyl and C1-C3 alkenyl, and R₁₀ is selected from a 5 to 10 membered heterocycle or a C6-C10 aryl, optionally further substituted with 1 to 4 membered group selected from alkyl, alkenyl, amide, carboxyl alkylcarbonyl, alkoxy and halogenide,
substituent S₃ being a polyether, preferably a polyethylene glycol, wherein the number ether —O— is 2 to 12, preferably 3 to 9, (or in an alternative embodiment R₁₆ is a group as defined for R₁₅
R₇ is H or a C1-C4 alkyl, a C6-C10 aryl or a 5 to 6 membered heterocycle, preferably heteroaryl, H, amine, preferably C1-C4 amide, amide preferably C1-C5 amide C2-C4 alkenyl, a a C1-C4 carbonyl, (preferably a C2-C4 alkylcarbonyl, C3-C4 alkenylcarbonyl, C3-C4 alkynylcarbonyl,) a C1-C4 carboxyl, (preferably a C2-C4 alkylcarboxyl, C3-C4 alkenylcarboxyl or C3-C4 alkynylcarboxyl), a C2-C5 carboxylate ester (preferably a C2-C5 alkylester, C3-C5 alkenylester or C3-C5 alkynylester) wherein if any of R₇ and R₈ is substituted, said substituent, if any, being selected from halogenide, pseudohalogenide, —OH, —SH, —OMe, —NO₂, —NH₂, —NHMe.

In a particular embodiment wherein R₂, R₃, R₄ and R₅ is selected from ureido (preferably phenyl-ureido optionally substituted with (preferably in para position) C1-C3 alkyl, C2-C4 alkenyl, C2-C4 alkynyl, C1-C4 alkoxy, halogenide, pseudohalogenide, —OH, —SH, —OMe, —NO₂, —NH₂, —NHMe, in particular C1-C2 alkoxy, halogenide, methyl or unsubstituted, highly preferably (para-metoxy-phenyl)-ureido), then
R₁₄ and R₁₅ are, independently, selected from H and C1-3, preferably C1-C2 alkyl, in particular methyl,
R₁₆ is selected from H, substituted or unsubstituted C1-C4 alkyl, C2-C4 alkenyl, C2-C4 alkynyl, C1-C4 alkoxy, —C(O)H, C2-C4 alkylcarbonyl, C3-C4 alkenylcarbonyl, C3-C4 alkynylcarbonyl, C1-C4 alkylamide, C1-C4 amide, carboxyl, C2-C4 alkylcarboxyl, a C3-C4 alkenylcarboxyl, a C3-C4 alkynylcarboxyl, and a C2-C4 carboxylate ester said substituent, if any, being selected from halogenide, pseudohalogenide, —OH, —SH, —OMe, —NO₂, —NH₂, —NHMe, preferably H, carboxyl and C2-C4 alkylcarboxyl,
   R₁₇, if present, is selected from H and a group as defined in paragraph 8; and
   R₄₂ is selected from the group consisting of H, C1-C30 alkyl, C2-C30 alkenyl, C2-C30 alkynyl, C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group,
said substituent C1-C4 alkyloxy group (in particular a C1-alcoxy, preferably a CH₂—O— moiety), if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe.

In a preferred embodiment said compound having general formula (I.1) or (I.1.1) has general formula (I.2) or (I.2.1), respectively,

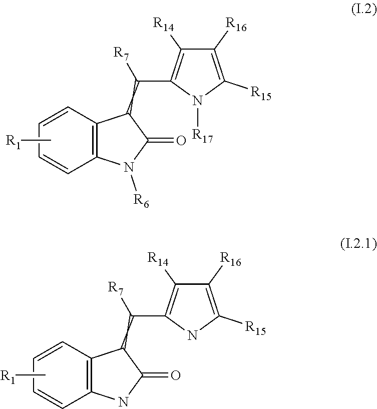

wherein R1 is an azido group
and any or each other substitutents are as defined in the present paragraph or in any numbered paragraph below.

Preferably the subject to be treated suffers from ocular neovascularization and/or the mechanism of action of the said compound is based on inhibiting the said ocular neovascularization. Preferably, the pharmaceutical composition is for use in the prevention or reduction of ocular neovascularization in the subject.

Preferably said ocular disease being selected from any other ocular disease where a targetable endogenous biomolecule is part of the pathomechanism.

Preferably the said ocular disease being selected from the group consisting of
macular degeneration, in particular age-related macular degeneration (AMD),
retinopathies, in particular diabetic retinopathies, proliferative retinopathies, e.g proliferative diabetic retinopathy (PDR),
macular oedema, in particular diabetic macular oedema (DME),
retinal vein occlusion (RVO),
open angle glaucoma (OAG),
angle closure glaucoma (ACG),
congenital glaucoma (CoG).

Preferably said ocular disease being selected from neurodegeneration in glaucoma, preferably neurodegeneration in OAG, ACG or CoG.

More preferably the said ocular disease involves neovascularization, preferably neovascularization that can be blocked by the inhibition of ocular VEGF signaling.
11. In a preferred embodiment the compound has general formula (II) or (11.1), preferably a compound for use according to paragraph 10,

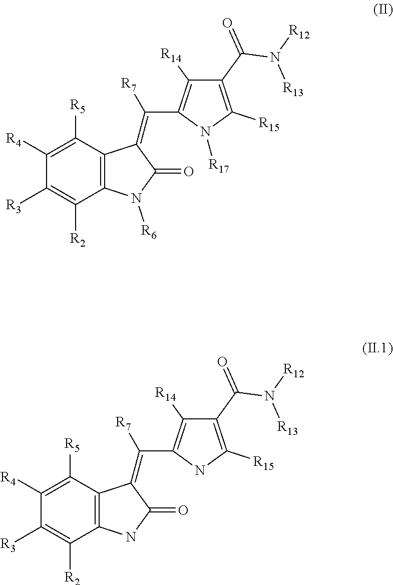

wherein in the formula R₂, R₃, R₄ and R₅ are selected from the group as defined above in paragraph 7, 8 or in paragraph 10, preferably paragraph 8 or 10 (preferably for formula X.1 or preferably for formula I.1),
wherein at least one of R₂, R₃, R₄ and R₅ is azido
   or preferably one of R₂, R₃, R₄ and R₅ is an azido group (N₃);
   wherein any one of R₂, R₃, R₄ and R₅ which is different from an azido group, is selected from the group consisting of
   —H, Me, halogenide, pseudohalogenide, —OH, —SH, —OMe, —OEt, —NO₂, —NH₂, —NHMe, —COOH, CONH₂—CF₃; preferably —H, halogenide, pseudohalogenide, —OMe, —OH, —SH,
   substituted or unsubstituted C1-C4 alkyl, C1-C4 alkenyl, C1-C4 alkoxy, C1-C4 alkylcarbonyl, C1-C4 alkenylcarbonyl, C6 aryl, C7-C8 alkylaryl (aralkyl), 5 to 6 membered heteroaryl, 6-8 membered alkyl-heteroaryl, said substituent, if any, being selected from halogenide, pseudohalogenide, —OH, —SH, —OMe, —NO₂, —NH₂, —NHMe, preferably halogenide, —OH, —OMe, —NH₂, —NHMe, preferably H and halogenide,
   NR₂₁R₂₂, wherein R₂₁ and R₂₂ are, selected from H, methanesulfonyl, ethanesulfonyl, phenylsulfonyl, substituted or unsubstituted C1-C3 alkyl and C1-C3 alkoxy, said substituent, if any, preferably being selected from halogenide, pseudohalogenide, —OH, —SH, —OMe, —NO₂, —NH₂, —NHMe, preferably —OH, —OMe, —NH₂, —NHMe and halogenide, more preferably halogenide, wherein preferably at least one of R₂₁ and R₂₂ is H, Me or Et, preferably H or Me,
   SO₂NR₂₃R₂₄, wherein R₂₃ and R₂₄ are, independently selected from H, substituted or unsubstituted C1-C3, C6 aryl, C7-C8 alkylaryl (aralkyl), 5 to 6 membered heteroaryl, 6-8 membered alkyl-heteroaryl, said substituent, if any, preferably being selected from halogenide, pseudohalogenide, —OH, —SH, —OMe, —NO₂, —NH₂, —NHMe, more preferably halogenide, wherein preferably at least one of R₂₃ and R₂₄ is H, Me or Et,
   phenyl-ureido optionally substituted in para position with C1-C3 alkyl, C2-C4 alkenyl, C2-C4 alkynyl, C1-C4 alkoxy, halogenide, pseudohalogenide, —OH, —SH, —OMe, —NO₂, —NH₂, —NHMe, in particular C1-C2 alkoxy, halogenide, methyl or unsubstituted, highly preferably (para-metoxy-phenyl)-ureido,
wherein R₁₄, R₁₅ and R₇ is as defined in paragraph 8 or 10, preferably as defined paragraph 10,
wherein preferably R₁₂ and R₁₃ is selected from C1-C4 or C2-C3 alkyl, preferably substituted with a substituent selected from one or two C1-C4 or C2-C3 alkyl or a polyether, preferably a polyethylene glycol, wherein the number ether —O— is 2 to 12, preferably 3 to 9, optionally a cyclic polyether forming a tertiary amine, (i.e. said amine being a secondary or tertiary amine), wherein optionally R₁₅ and R₁₃ or R₁₄ (together with the backbone atoms) form a 5 to 8 membered heterocycle, preferably a 5 or 6 to 7 membered, preferably a 5 or 6 membered, in particular a 6 membered heterocycle.

In a preferred embodiment in formula (II)
one of R₂, R₃, R₄ and R₅ is azido,
and one or two preferably one of R₂, R₃, R₄ and R₅ is a halogen, pseudohalogen, OH, or OMe, preferably a halogen
and the other one or two preferably two of R₂, R₃, R₄ and R₅ is H.

Preferably, R₁₄, R₁₅ and R₇ is as defined in paragraph 10, preferably
R₁₄ is selected from H and a C1-C3 alkyl or C2-C3 alkenyl preferably wherein the π electron pair is conjugated with the π electron system of the pyrrole ring,
R₁₅ is selected from H and a C1-C3 alkyl or C2-C3 alkenyl preferably wherein the π electron pair is conjugated with the π electron system of the pyrrole ring,
R₇ is H or a C6-C10 aryl or a 5 to 10 membered heterocycle, preferably heteroaryl
R₁₂ and R₁₃ is selected from H, C1-C8 alkyl (preferably C1-C4 or C2-C3 alkyl) preferably substituted with a substituent selected from one or two C1-C4 or C2-C3 alkyl or a polyether, preferably a polyethylene glycol, wherein the number ether —O— is 2 to 12, preferably 3 to 9, optionally a cyclic polyether forming a tertiary amine, (i.e. said amine being a secondary or tertiary amine), wherein optionally R₁₅ and R₁₃ or R₁₄ (together with the backbone atoms) form a 5 to 8 membered heterocycle, preferably a 5 or 6 to 7 membered, preferably a 5 or 6 membered, in particular a 6 membered heterocycle, or amine; said amine being optionally substituted with one or two C1-C4 or C2-C3 alkyl or a group as defined as substituent S₃ below, optionally a cyclic polyether forming a tertiary amine, (i.e. said amine being a secondary or tertiary amine),
R₆ and R₁₇ are, independently, as defined in paragraph 8 or 9, or preferably,
   R₆ is selected from H and a substituted or unsubstituted C1-C4 alkyloxy group (in particular a C1-alcoxy, preferably a CH₂—O— moiety) preferably acylated to be an ester by an —C(O)—R₃₁ group, wherein R₃₁ is selected from the group consisting of OR₃₂, SR₃₂, and N(R₃₂)₂; and
   R₃₁ and/or R₃₂ is/are, independently, as defined in paragraph 8 or 9,
   or in a particularly preferred embodiment R₆ is a biotinyl group
   R₁₇, if present, is selected from H and an intracellularly metabolizable group; preferably R₁₇, once present, is a substituted or unsubstituted C1-C4 alkyloxy group (in particular a C1-alcoxy, preferably a CH₂—O— moiety) preferably acylated to be an ester by an —C(O)—R₄₁ group, wherein R₄₁ is selected from the group consisting of OR₄₂, SR₄₂, and N(R₄₂)₂; and
   R₃₁ and/or R₄₂ is/are, independently, as defined in paragraph 8 or 9
   or in a particularly preferred embodiment R₆ comprises a biotinyl group.
12. The invention relates to a compound for use according to any of paragraphs 10 to 11 for use in the treatment of a disease as defined in paragraph 9.
13. The invention relates to a compound for use according to any of paragraphs 10 to 11 for use in the treatment of an ocular disease selected from the group consisting of
   macular degeneration, in particular age-related macular degeneration (AMD),
   retinopathies, in particular diabetic retinopathies, proliferative retinopathies, e.g proliferative diabetic retinopathy (PDR),
   macular oedema, in particular diabetic macular oedema (DME),
   retinal vein occlusion (RVO),
   open angle glaucoma (OAG),
   angle closure glaucoma (ACG),
   congenital glaucoma (CoG).

Preferably said ocular disease being selected from neurodegenerative conditions in glaucoma, preferably neurodegeneration in OAG, ACG or CoG.

Preferably said ocular disease being selected from any other ocular disease
that involves neovascularization, preferably neovascularization that can be blocked by the inhibition of ocular VEGF signaling.

In a particular embodiment the invention relates to a compound for use according to any of paragraphs 10 to 11 for use in the treatment of an ocular disease where ocular neovascularization is part of the pathomechanism. In a particular embodiment the invention relates to a compound for use according to any of paragraphs 10 to 11 for use in the prevention or reduction of ocular neovascularization in a mammalian subject.

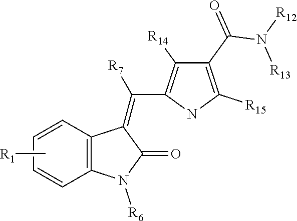

14. In a preferred embodiment the compound for use according to any of paragraphs 1 to 13, said compound having general formula selected from the group consisting of general formulae (V.1), (VI.1), (VII.1) and (VIII.1):

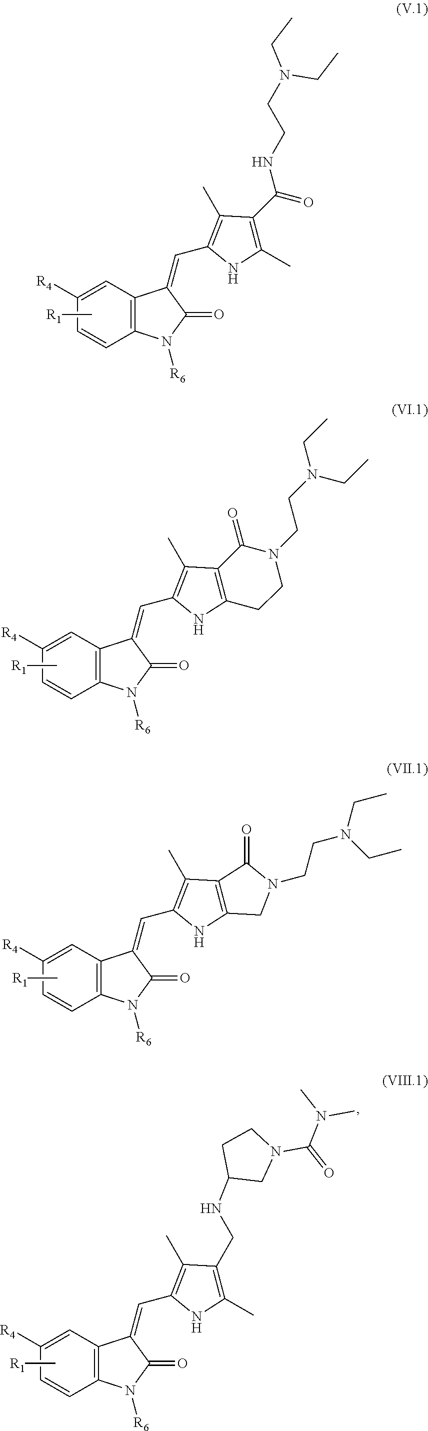

or in an embodiment said compound having general formula selected from the group consisting of general formulae (V), (VI), (VII) and (VIII):

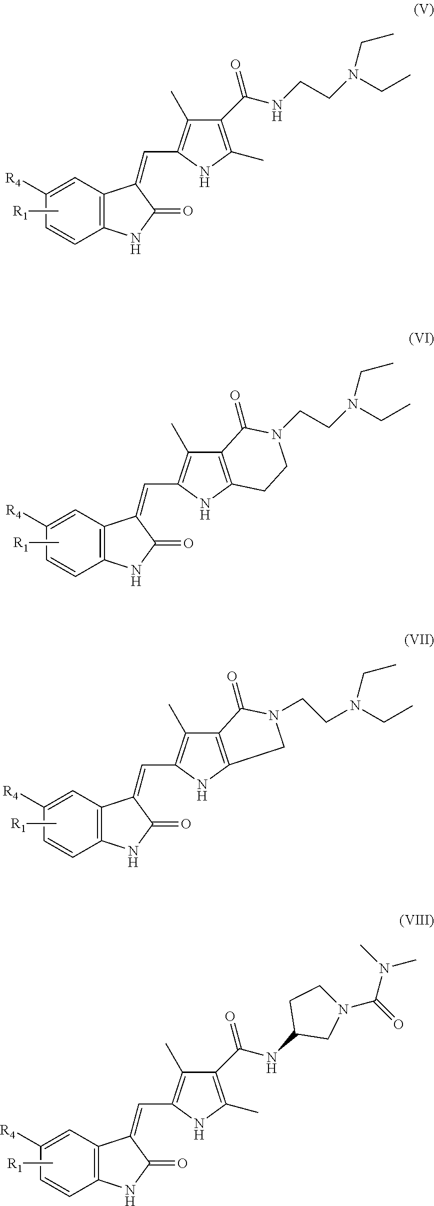

wherein R₁ is an azido group connected to carbon 4, 6 or 7 of the indole-2-one moiety, and
R₄ is selected from the group consisting of
H, Me, halogenide, pseudohalogenide, —OH, —SH, —OMe, —OEt, —NO₂, —NH₂, —NHMe, —COOH, CONH₂—CF₃; preferably H, halogenide, pseudohalogenide, —OMe, —OH, —SH, in particular halogenide, highly preferably F or Cl; and
in preferred embodiments, R₆ group on the nitrogen atom of the indoline-2-one moiety is present to create a prodrug and is as defined in paragraphs 8, 9, 10 or 11.

In still alternative, less preferred embodiments, R₁₇ group is present on the nitrogen atom of the indoline-2-one moiety may be present and is as defined in paragraphs 8, 9, 10 or 11.
15. In a preferred embodiment the compound for use according to any of paragraphs 1 to 13, said compound having general formula selected from the group consisting of general formulae (V.1), (VI.1), (VII.1) and (VIII.)
or in an embodiment general formulae (V), (VI), (VII) and (VIII):
wherein R₁ is connected to carbon 4, 6 or 7 of the indole-2-one moiety and is selected from the group consisting of H, Me, halogenide, pseudohalogenide, —OH, —SH, —OMe, —NO₂, —NH₂, —NHMe, in particular H and halogenide, highly preferably F or Cl and
R₄ is an azido group, and
in preferred embodiments, R₆ group on the nitrogen atom of the oxindole moiety is present to create a prodrug and is as defined in paragraphs 8, 9, 10 or 11.

In still alternative less preferred embodiments, R₁₇ group on the nitrogen atom of the oxindole moiety is present and is as defined in paragraphs 8, 9, 10 or 11.
16. In a preferred embodiment the compound for use according to any of paragraphs 1 to 13, obtained by modifying benzene ring of the PMO in sunitinib and is selected from the following compounds having formula (1), (2), (3), (4), (5), and (6), that were also synthetized to provide a proof-of-concept of the invention (FIGS. 2-7) or compounds having the same formula but having an R₆ group on the nitrogen atom of the oxindole moiety to create a prodrug and said R₆ group being defined in any of paragraphs 8, 9, 10 or 11.

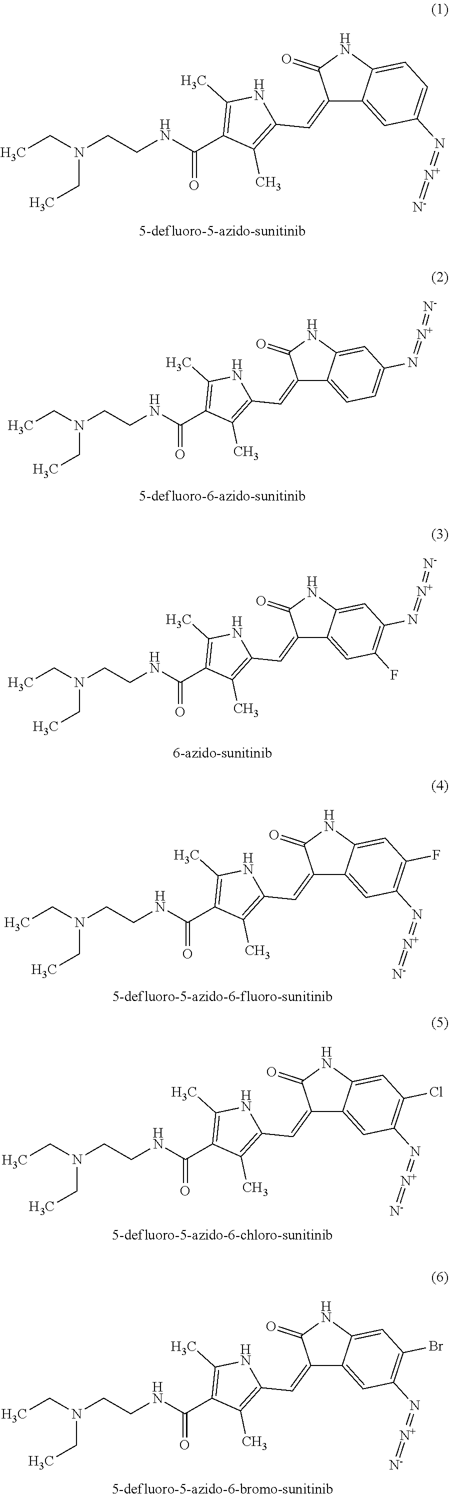 [SMILES: CCN(CC)CCNC(=O)c1c(C)[nH]c(/C=C2\C(=O)Nc3ccc(N=[N+]=[N-])cc32)c1C ; CCN(CC)CCNC(=O)c1c(C)[nH]c(/C=C2\C(=O)Nc3cc(N=[N+]=[N-])ccc32)c1C ; CCN(CC)CCNC(=O)c1c(C)[nH]c(/C=C2\C(=O)Nc3cc(N=[N+]=[N-])c(F)cc32)c1C ; CCN(CC)CCNC(=O)c1c(C)[nH]c(/C=C2\C(=O)Nc3cc(F)c(N=[N+]=[N-])cc32)c1C ; CCN(CC)CCNC(=O)c1c(C)[nH]c(/C=C2\C(=O)Nc3cc(Cl)c(N=[N+]=[N-])cc32)c1C ; CCN(CC)CCNC(=O)c1c(C)[nH]c(/C=C2\C(=O)Nc3cc(Br)c(N=[N+]=[N-])cc32)c1C]

17. In a preferred embodiment the compound for use according to any of paragraphs 1 to 13, preferably obtained by modifying benzene ring of the PMO in vorolanib and is selected from the following compounds that were also synthetized to provide a proof-of-concept of the invention (FIGS. 8-9), and compounds having the same formula but having an R₆ group on the nitrogen atom of the oxindole moiety to create a prodrug and said R₆ group being defined in any of paragraphs 8, 9, 10 or 11:

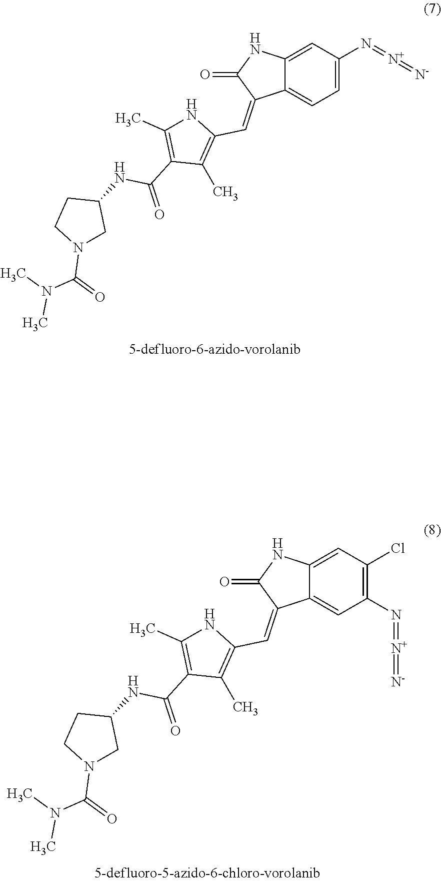 [SMILES: Cc1[nH]c(/C=C2\C(=O)Nc3cc(N=[N+]=[N-])ccc32)c(C)c1C(=O)N[C@H]1CCN(C(=O)N(C)C)C1 ; Cc1[nH]c(/C=C2\C(=O)Nc3cc(Cl)c(N=[N+]=[N-])cc32)c(C)c1C(=O)N[C@H]1CCN(C(=O)N(C)C)C1]

18. In a preferred embodiment said compound is capable of binding to the biological target (preferably a receptor or an enzyme) in an assay, preferably in vitro.
19. In a preferred embodiment said compound is capable of inhibiting VEGFR2 in a VEGFR2 inhibition assay, preferably in vitro.

In an embodiment the compound is capable of modulating, preferably inhibiting multiple kinases, i.e. preferably is a multikinase inhibitor; wherein said kinases are preferably biological targets as defined herein.

These functional features can be tested by a skilled person and a particularly useful compound can be selected by a person skilled in the art.
20. The invention also relates to a pharmaceutical composition, including but not limited to oral compositions for ophthalmic use said composition comprising a compound as defined in any one of paragraphs 1 to 19, in particular in any one of paragraphs 8, 10, 11, 14, 16 or 17 and a pharmaceutically acceptable excipient. Preferably said oral compositions are protected from ambient light in their packaging.

The invention also relates to a pharmaceutical composition, including but not limited to eyedrop compositions for ophthalmic use, the said composition comprising a compound as defined in any one of paragraphs 1 to 19, in particular in any one of paragraphs 8, 10, 11, 14, 16 or 17 and a pharmaceutically acceptable excipient. The said eyedrop compositions are protected from ambient light in their packaging. The active compound in of the eyedrop formulation is also protected from light when applied on the corneal surface by using (i) a fully solubilized complex with a photoprotective cyclodextrin, (ii) a suspension with photoprotective liposomes, (iii) or an equivalent solution that serves the protection of the active product ingredient from light.
21. The invention preferably relates to an oral pharmaceutical composition for ophthalmic use, said pharmaceutical composition being protected from light. In one possible embodiment a non-transparent capsule protects the formulation from light. In a further embodiment a photoprotective liposome or a photoprotective cyclodextrin protects the compound.
22. The invention preferably relates to an oral pharmaceutical composition for use according to any of paragraphs 20 to 21 against a disease as defined in any of paragraphs 1 to 9, preferably paragraph 4, 9 or 13, preferably paragraph 9.
23. In a preferred embodiment the pharmaceutical composition is for use in the treatment of an ocular disease where a targetable ocular biomolecule is part of the pathomechanism, preferably as defined in any one of the respective paragraphs herein.

Preferably, the pharmaceutical composition is for use in the prevention or reduction of ocular neovascularization in a subject.

Preferably the invention relates to the pharmaceutical composition for use according to any of paragraphs 8, 10, 11, 14, 16 or 17 in particular any of paragraphs 10, 11, 16, 17 for use in the treatment of an ocular disease selected from the group consisting of
macular degeneration, in particular age-related macular degeneration (AMD),
retinopathies, in particular diabetic retinopathies, proliferative retinopathies, e.g proliferative diabetic retinopathy (PDR),
macular oedema, in particular diabetic macular oedema (DME),
retinal vein occlusion (RVO).

In an embodiment the ocular disease is glaucoma.
24. The invention also relates to a method of treating a patient in need of ophthalmic treatment, having a disease as defined in any of the above paragraphs, preferably in any of paragraphs 4, 9 and 13 comprising administering the pharmaceutical composition to said patient.

In an embodiment the patient's eye is illuminated after administration by ambient light.

The invention also relates to and teaches a method for treatment of a disease as defined in paragraph 9 in a subject in need thereof comprising
administering orally a compound as defined in any of paragraphs 1 to 18, preferably in paragraph 8, 10, 11, 16 or 17 or a pharmaceutical composition according to any of paragraphs 20 to 22,
allowing ambient light entering the eye to photoactivate said compound and elicit the covalent binding of the said compound to the ocular target biomolecule.

The invention also relates to a method of treating a patient in need of ophthalmic treatment, having a disease as defined in any of the above paragraphs, preferably in any of paragraphs 4, 9 and 13 comprising administering the orally formulated pharmaceutical composition orally to said patient.

The invention also relates to a method of treating a patient in need of ophthalmic treatment, having a disease as defined in any of the above paragraphs, preferably in any of paragraphs 4, 9 and 13 comprising administering the pharmaceutical composition, formulated as an eye-drop, to said patient via direct application onto the eye.

The invention also relates to and teaches a method for treatment of a disease as defined in paragraph 4, 9, 13 or 23, preferably in paragraph 9 in a subject in need thereof comprising:
administering an eyedrop formulation that comprises the azidated compound and a photoprotective coating (such as a liposome, a cyclodextrin or an equivalent photoprotective agent) to target the said compound to the desired part of the eye.
allowing enough time for the photoprotected active product ingredient to leave the surface of the eye, to be absorbed and to accumulate in the targeted ocular tissue
allowing ambient light entering the eye to photoactivate said compound an elicit the covalent binding of the said compound to the ocular target biomolecule.
25. The method for treatment as defined in paragraph 24 wherein the ambient light is provided artificially, preferably by illumination by a light source providing polychromatic light.

Preferably the light has a spectrum having a wavelength of at least 350 nm, preferably at least 400 nm, and up to 800 nm, preferably up to 600 nm or as defined herein.

Preferably the light has a spectrum of at least 100 nm wide, preferably at least 200 nm wide, in a wavelength-range spanning from at least 350 nm, preferably at least 400 nm, and up to 800 nm, preferably up to 600 nm.

Preferably the light is or comprises visible light.

Preferably the light is white light.
26. The invention also relates to and teaches a method of treatment wherein the light is provided artificially.

In an embodiment the light is provided by a wearable apparatus.

In an embodiment the light is provided by an apparatus which can engage the subject.

In an embodiment the light is provided in a room wherein the subject is present.
27. According to the invention, preferably in any one of paragraphs 1 to 26, the subject is a vertebrate subject, preferably a mammalian subject. Highly preferably the subject is a human subject.

Preferably the subject is a patient diagnosed with an ocular disease.

Preferably the patient is diagnosed with a disease as defined in paragraph 9.

Preferably the patient is diagnosed with a disease as defined in paragraph 23.
28. A compound having general formula (I.1) or (I.1.1)

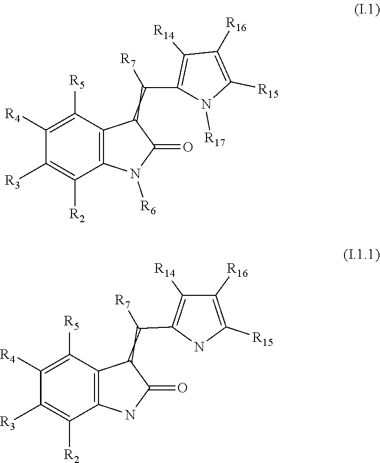

wherein in the formula
R₂, R₃, R₄, R₅ R₇, R₁₄, R₁₅ and R₁₆ are as defined in paragraph 10,
preferably, where appropriate, as defined in paragraph 11.

Preferably said compound having general formula (I.1) has general formula (I.3) or (I.1.1) has general formula (I.3.1),

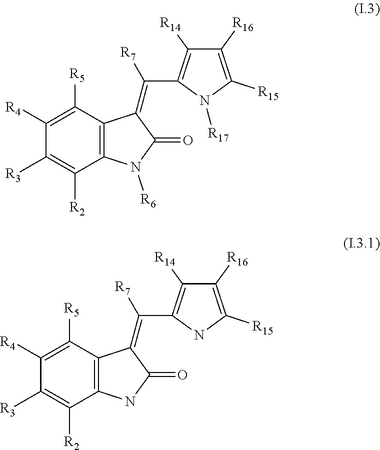

i.e. compound comprising a “pyrrol-methylidene-oxindole” (3-[(1H-pyrrol-2-yl)methylidene]-1,3-dihydro-2H-indol-2-one) moiety,
wherein any or each of the substituents are R₂, R₃, R₄, R₅ R₇, R₁₄, R₁₅ and R₁₆ are as defined in paragraph 10.
preferably, where appropriate, as defined in paragraph 11.

In a preferred embodiment said compound having general formula (I.1) has general formula (I.2),

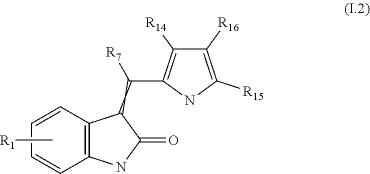

wherein R₁ is an azido group
and any or each other substituents are as defined in the present paragraph or in any numbered paragraph below.
29. The compound according paragraph 28, wherein said compound has general formula (II) or (II.1)

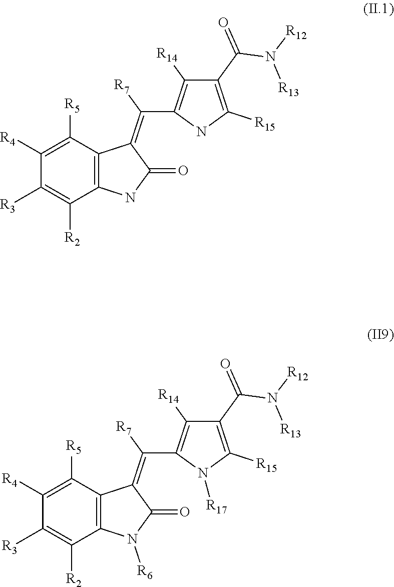

wherein R₂, R₃, R₄, R₅, R₇, R₁₂, R₁₃, R₁₄, R₁₅ and R₁₆ are as defined in paragraph 11.
30. The compound according to any of paragraphs 28 to 29, said compound having general formula selected from the group consisting of general formulae (V.1), (VI.1), (VII.1) and (VIII.1) or general formulae (V), (VI), (VII) and (VIII) as defined in paragraph 14, preferably
wherein R₁ is an azido group connected to carbon 4, 6 or 7 of the indole-2-one moiety, and
R₄ is selected from the group consisting of
H, Me, halogenide, pseudohalogenide, —OH, —SH, —OMe, —OEt, —NO₂, —NH₂, —NHMe, —COOH, CONH₂—CF₃; preferably H, halogenide, pseudohalogenide, —OMe, —OH, —SH, in particular halogenide, highly preferably Cl or F; and
in preferred embodiments, R₆ group on the nitrogen atom of the indoline-2-one moiety is present to create a prodrug and is as defined in paragraphs 8, 9, 10 or 11.
31. The compound according to any of claims 28 to 29, said compound having general formula selected from the group consisting of general formulae (V.1), (VI.1), (VII.1) and (VIII.1) or general formulae (V), (VI), (VII) and (VIII) as defined in paragraph 14, preferably
wherein R₁ is connected to carbon 4, 6 or 7 of the indole-2-one moiety and is selected from the group consisting of H, Me, halogenide, pseudohalogenide, —OH, —SH, —OMe, —NO₂, —NH₂, —NHMe, in particular H and halogenide, highly preferably Cl or F and
R₄ is an azido group.
32. The compound according to any of paragraphs 28-29, preferably obtained by modifying benzene ring of the PMO in sunitinib, wherein said compound is selected from the following compounds:
5-defluoro-5-azido-sunitinib, shown on formula (1),
5-defluoro-6-azido-sunitinib, shown on formula (2),
6-azido-sunitinib, shown on formula (3),
5-defluoro-5-azido-6-fluoro-sunitinib, shown on formula (4),
5-defluoro-5-azido-6-chloro-sunitinib, shown on formula (5),
5-defluoro-5-azido-6-bromo-sunitinib, shown on formula (6),
that were also synthetized to provide a proof-of-concept of the invention (FIGS. 2-7):
33. The compound according to any of paragraphs 28-29, preferably obtained by modifying benzene ring of the PMO in vorolanib, said compound being selected from the following compounds that were also synthetized to provide a proof-of-concept of the invention (FIGS. 8-9):
(7) 5-defluoro-5-azido-vorolanib, shown on formula (7)
(8) 5-defluoro-5-azido-6-chloro-vorolanib, shown on formula (8).

Preferably, unless otherwise defined herein,
C1-C8 may be C1-C6, in particular C1-C4, highly preferably C1-C3,
C2-C8 may be C2-C6, in particular C2-C4, highly preferably C2-C3, and
C3-C8 may be C3-C6, in particular C3-C4, highly preferably C3,
throughout the paragraphs above or the claims.
34. Use of a compound described in any of paragraphs 1 to 17 or a compound according to any of paragraphs 23 to 33 in an assay, said compound binding to the biological target (preferably a receptor or an enzyme) preferably in vitro.
35. Use of a compound described in any of paragraphs 1 to 17 or a compound according to any of paragraphs 23 to 33 in an assay said compound inhibiting VEGFR2 in a VEGFR2 inhibition test, preferably in vitro.

### Definitions

A “subject” as used herein is an individual of an animal species, preferably a vertebrate, more preferably a mammalian or avian species, in particular a mammalian species, highly preferably the individual is a primate, a hominid or a human. A “patient” is a subject who is or intended to be under medical or veterinarian observation, supervision, diagnosis or treatment.

A “treatment” of a subject refers to any process, action, therapy, or the like, wherein the subject or patient is under aid, in particular medical or veterinarian aid with the object of improving the subject's or patient's condition, either directly or indirectly. Improving the subject's condition may include restoring or maintaining normal function of an organ or tissue, preferably at least partly restoring or maintaining health (medical or veterinarian treatment). Treatment typically refers to the administration of an effective amount of a compound or composition described herein. In a broader sense treatment includes both medical or veterinarian treatment and prevention (or prophylaxis) i.e. prevention of the onset of a disease as well, in a more limited sense prevention is not covered.

“Ambient light” in accordance with the invention is the light useful for illumination of the eye of the patient which is provided by a natural or artificial light source and which is the light actually seen by the patient and which illuminates the area of retina onto which light is projected by the optical apparatus of the eye; said light is polychromic and always covers a wavelength range including the wavelength activating the compounds of the invention. The ambient light is different from a coherent monochromatic light in particular a laser light. In a preferred embodiment the ambient light is not artificially focused (i.e. is non-directed); preferably it is not focused or directed on the retina artificially. In an embodiment the light is or comprises visible light. In an embodiment the light is white light. Preferably “white light” as used herein is a combination of lights of different wavelengths, preferably a polychromatic light having a spectrum of at least 100 nm, preferably at least 200 nm in particular at least 300 nm wide in the range of at least 350 nm and up to 800 nm. Particular ranges are defined in the Brief description of the invention an in the appended claims.

A “pharmaceutical composition” of the invention is a composition of matter which comprises at least one compound of the invention comprising an active agent and at least one further substance. Preferably the compound of the invention is present in an effective amount. Compositions may also comprise further biologically active substances useful e.g. in a combination therapy. Furthermore, the compositions may comprise biologically acceptable carriers, formulation agents, excipients etc. which may be known in the art.

The term “effective amount” qualifies the amount of a compound required to exert the effect of the active agent in a composition. A “therapeutically effective amount” is sufficient to relieve or prevent (or prevent worsening of) one or more of the symptoms or characteristic parameters of a condition, e.g. a disorder or disease.

A “moiety” is used herein as a part of a molecule which can be derived in principle by removing another part, even a hydrogen atom or a group or any part thereof.

An “active agent moiety” as used herein is a part of the compound of the invention which carries the biological effect of said compound and which is capable of specifically binding to its biological target molecule. The active agent moiety carries this biological effect even if present in a separate (non-azidated) molecule. The said biological effect includes partial or full inhibition and partial or full activation of the biological target molecule.

A “conjugated moiety” is a part of the compound of the invention which carries a conjugated system even without the azido group.

In an embodiment the active agent moiety is the conjugated moiety itself i.e. “conjugated active agent moiety”.

In an embodiment the conjugated moiety is bound to an active agent moiety. For example it may serve as a linker between the azide moiety and the active agent moiety.

A “parental molecule” as used herein is a molecule from which the compound of the invention can be derived by azidation; in particular the parental molecule is a non-azidated counterpart of the compound of the invention. In an embodiment parental molecule is formed by or consists of the active agent moiety and the conjugated moiety. In an embodiment parental molecule is formed by or consists of the conjugated active agent moiety, or the active agent moiety.

The term “PMO” is used herein to refer to a molecular substructure “pyrrol-methylidene-oxindole”, formally named 3-[(1H-pyrrol-2-yl)methylidene]-1,3-dihydro-2H-indol-2-one that is a common element of numerous receptor tyrosine kinase inhibitors such as semaxanib, sunitnib, toceranib, vorolanib, famitinib.

A “prodrug” according to the invention is a compound which is a derivative of an active agent, e.g. biologically active molecule, e.g. a medicament, which can be administered to a subject and which is metabolized to an active agent in the subject. Typically a prodrug comprises a functional group that renders the active agent inactive and the said functional group can be cleaved off to release the original active agent or can be metabolized into the original functional group in the body of the subject, the chemical derivatization moiety being characterized as a “metabolizable group”.

As used herein, the term “alkyl” alone or in combinations means a straight or branched-chain (if appropriate) hydrocarbon group containing preferably 1 to 15, 1 to 10 or 1 to 8 carbon atom(s) or in particular 1 to 6 or 1 to 4, 1 to 3 or 1 to 2 carbon atom(s) [i.e. “C₁₋₁₅”, “C₁₋₁₀”, “C_{1_8}”, “C₁₋₆” or in particular “C₁₋₆”, “C₁₋₄”, “C₁₋₃” or “C₁₋₂” alkyl groups, or lower alkyl, respectively], such as particularly preferably methyl, ethyl, propyl or isopropyl groups.

As used herein, the term “alkoxy” means an alkyl-O— group in which the alkyl group is as previously described. The bond to the rest of the molecule or complex, i.e. the parent moiety is through the oxygen (if to a carbon atom, ether oxygen).

The term “alkoxy alkyl” means an alkyl group which is substituted by an alkoxy group, i.e. an alkyl-O— group as previously described. The bond to the alkyl moiety is through the oxygen, i.e. it is an ether oxygen.

As used herein, the terms “carbonyl”, “alkyl-carbonyl”, “alkenyl-carbonyl” and “alkynyl-carbonyl” mean a moiety having carbonyl group optionally substituted with an alkyl group, alkenyl group and alkynyl group, respectively. In a wider sense the group can be connected by either the alkyl, alkenyl or alkynyl or via the carbonyl group. In a preferred embodiment, i.e. narrower sense, the group bond to the parent moiety is through the carbon of the carbonyl group. In a preferred embodiment the “alkyl-carbonyl”, “alkenyl-carbonyl” and “alkynyl-carbonyl” is alkanoyl, alkenoyl and alkynoyl, respectively.

This definition of wider sense and narrower sense pertains to any analogous groups with a functional group used herein even if not defined separately.

As used herein, the terms “carboxyl”, “alkyl-carboxyl”, “alkenyl-carboxyl” and “alkynyl-carboxyl” are defined to mean a moiety having carboxyl group optionally substituted with an alkyl group, alkenyl group and alkynyl group, respectively, wherein bond to the parent moiety is through the carboxyl group. The group can be connected by either the alkyl, alkenyl or alkynyl or via the carboxyl group (in the latter case being an esther).

An “alkenyl” as used herein, alone or in combinations, means a straight or branched-chain unsaturated hydrocarbon group containing at least one carbon-carbon double bond, said hydrocarbon group containing preferably from 2 to 20, preferably 2 to 15, 2 to 10 or 2 to 8 carbon atoms or 2 to 6, 2 to 4, 2 to 3 or 2 carbon atoms [i.e. “C₂₋₂₀”, “C₂₋₁₅”, “C₂₋₁₀”, “C₂₋₈”, “C₂₋₆” or “C₂₋₄”, “C₂₋₃” or “C₂” alkyl groups, respectively or in particular “C₂₋₆”, “C₂₋₄”, “C₂₋₃” or “C₂” alkenyl groups or lower alkenyl, respectively].

An “alkynyl” as used herein is defined analogously to alkenyl mutatis mutandis.

A “heterocyclic” ring as used herein is a cyclic moiety that has, besides carbon atom(s), atoms of at least one non-carbon element as member(s) of its ring(s). A heterocycle may comprise multiple rings, e.g. it may comprise an aromatic heterocycle and, fused to the aromatic heterocycle another ring which may or may not be aromatic; i.e. if it is not aromatic it may form a cyclic substituent of the aromatic heterocycle. In a preferred embodiment, if the heteroaryl comprises multiple, in particular two fused rings, both rings are aromatic. Preferably the ring(s) of the heterocyclic moiety is/are 5 to 6 membered ring(s).

A “heterocyclyl” group is a group comprising a heterocyclic moiety, preferably one or more, e.g. one or two “heterocyclic” ring, which may be substituted or unsubstituted; is substituted, without limitation, it may be substituted with a functional group, e.g. an oxo, hydroxyl, amino, halogen, nitro, carboxyl, lower alkyl, alkenyl or alkyinil, etc.

The term “heterocycloalkyl” refers to a “heterocyclic” ring which is derivable from cycloalkyl group as defined above, wherein at least one of the carbon atoms of the ring is replaced with a heteroatom such as, but not limited to, nitrogen or oxygen.

An “aromatic” moiety as used herein can be described as a planar cyclic moiety (a ring) wherein the single bonds (called σ-bonds) between the ring-forming atoms are formed from overlap of hybridized atomic sp²-orbitals in line between the carbon nuclei, wherein a system of delocalized π-bonds are formed from overlap of atomic p-orbitals of each of the ring forming atoms above and below the plane of the ring and wherein the number of π electrons, which is provided by the ring-forming atoms, participates in according to molecular orbital theory, must be equal to 4n+2 (Hückel's rule), in which n=1, 2, 3, etc., preferably 1 or 2, for a single ring with six π electrons, n=1. The ring-forming atoms typically provide one or two π electrons to the delocalized π electron system.

A “conjugated system” as used herein can be described as a planar moiety having a carbon and/or heteroatom skeleton wherein the single bonds (called σ-bonds) are formed from overlap of hybridized atomic sp²-orbitals in line between skeleton carbon or heteroatom nuclei of the system, wherein a system of delocalized π-electrons are formed from overlap of atomic p-orbitals of each of the skeleton atoms above and below the plane of the ring from π electrons in one or more double bond(s), non-binding (lone) pair(s) (and in some cases radical electron(s) or electrons of carbenium ion(s)) to form an interrelated delocalized π electron system. Preferably the conjugated system comprises an aromatic system like that of an aryl moiety, to which preferably the azide moiety is linked.

In a conjugated π-system, electrons are able to capture photons. Typically, the more extended the conjugated π-system is, the longer the wavelength of photon can be captured.

Preferably a conjugated system of the invention comprises at least 3, preferably 4, more preferably at least 5 non-sigma electron pairs without (excluding) the π electron pair of the azide.

The term “heteroaryl” is defined herein as a group or molecule that contains an aromatic heterocycle, preferably a moiety that has at least one heteroatom, as “member”, incorporated within an aromatic ring. Examples of heteroatoms include nitrogen, oxygen and sulfur, preferably nitrogen and oxygen. In an embodiment a heteroaryl may comprise an aromatic heterocycle and, fused to the aromatic heterocycle another ring which may or may not be aromatic; i.e. if it is not aromatic it may form a cyclic substituent of the aromatic heterocycle. In a preferred embodiment, if the heteroaryl comprises multiple, in particular two fused rings, both rings are aromatic. Members of a heteroaryl relate to the ring-forming atoms, either carbon atom(s) or heteroatom(s).

The term “aryl” as used herein is a group that contains any carbon-based aromatic ring which is preferably a mono- or bicyclic group, wherein the bicyclic group preferably comprises two fused rings. In a preferred embodiment the aryl group consists of carbon as ring atoms, i.e. “members” only. In a broader meaning the term aryl also includes optionally “heteroaryl”. Optionally, the term “aryl” is limited to non-heteroaryl which is also included into the term aryl and defines a group that contains an aromatic group that does not contain a heteroatom.

An aryl group may be substituted or unsubstituted (i.e. optionally substituted). If the aryl group is substituted it may be substituted with any substituent, and examples of the substituent include C₁₋₄ alkyl, C₂₋₄ alkenyl, C₁₋₃ alkyloxy, C₁₋₃ alkanoyl, C₁₋₃ alkylamine, C₁₋₃ alkylamide, halogen, etc.

The term “aralkyl” as used herein refers to an aryl alkyl group which is linked to the parent molecule through the alkyl group, which may be further optionally substituted with one or more, preferably one to three or one to two alkyl substituents. Thus, the aryl group may be substituted with an alkyl substituent, preferably each substituent being not larger than a C₁₋₄ alkyl.

“Aryl” or “heteroaryl” may comprise a monocyclic ring, a condensed ring, or a polycyclic ring in which a single ring is bounded by a single bond, preferably a monocyclic or bicyclic ring.

As used herein, the term “fused ring” means that the ring is fused with at least one other ring to form a group of a compound which comprises two or more rings wherein a single bond between two member atoms of the rings is, together with said two members, common in, i.e. shared by the two rings. An example of fused rings is a polycyclic aryl. A polycyclic aryl is understood herein as a group that contains multiple rings of a carbon-based group among which at least one ring is an aryl and which optionally may also comprise a cycloalkyl and/or a heterocycloalkyl.

A “substituted” moiety comprises a substituent selected from the groups and moieties as defined herein; however, a substituent is preferably smaller, i.e. shorter, i.e. consists of not more, preferably less atoms than the moiety which is/are substituted thereby. In the present invention, “optionally substituted”, i.e. “unsubstituted or substituted” means that it may be substituted with any substituent.

In general formulae of the description H atoms are typically not shown, however, a skilled person is able to understand said formulae and recognize the full structure.

The singular forms “a”, “an” and “the”, or at least “a”, “an”, include plural reference unless the context clearly dictates otherwise.

The term “comprises” or “comprising” or “including” are to be construed here as having a non-exhaustive meaning and allow the addition or involvement of further features or method steps or components to anything which comprises the listed features or method steps or components. “Comprising” can be substituted by “including” if the practice of a given language variant so requires or can be limited to “consisting essentially of” if other members or components are not essential to reduce the invention to practice.

### BRIEF DESCRIPTION OF THE DRAWINGS/AZ ÁBRÁK RÖVID LEÍRÁSA

FIG. 1.: Illustration of the principle of the invention

FIG. 1a: The optical system of the eye processing the incoming ambient light

FIG. 1b: A magnified portion of the FIG. 1 illustrating the transient binding of the azidated compound to the receptor in the retina, activation of the compound by ambient light and covalent binding of the compound to the receptor it was transiently associated to

FIG. 2.: UV-VIS spectrum of 5-defluoro-5-azido-sunitinib

FIG. 3.: UV-VIS spectrum of 5-defluoro-6-azido-sunitinib

FIG. 4.: UV-VIS spectrum of 6-azido-sunitinib

FIG. 5.: UV-VIS spectrum of 5-defluoro-5-azido-6-fluoro-sunitinib

FIG. 6.: UV-VIS spectrum of 5-defluoro-5-azido-6-chloro-sunitinib

FIG. 7.: UV-VIS spectrum of 5-defluoro-5-azido-6-bromo-sunitinib

FIG. 8.: UV-VIS spectrum of 5-defluoro-6-azido-vorolanib

FIG. 9.: UV-VIS spectrum of 5-defluoro-5-azido-6-chloro-vorolanib

FIG. 10.: Inhibitory effect of the parental compound sunitinib on VEGFR2-HEK cells

FIG. 11.: Inhibitory effect of 5-defluoro-5-azido-sunitinib on VEGFR2-HEK cells

FIG. 12.: Inhibitory effect of 5-defluoro-6-azido-sunitinib on VEGFR2-HEK cells

FIG. 13.: Inhibitory effect of 6-azido-sunitinib on VEGFR2-HEK cells

FIG. 14.: Inhibitory effect of 5-defluoro-5-azido-6-fluoro-sunitinib on VEGFR2-HEK cells

FIG. 15.: Inhibitory effect of 5-defluoro-5-azido-6-chloro-sunitinib on VEGFR2-HEK cells

FIG. 16.: Inhibitory effect of 5-defluoro-5-azido-6-bromo-sunitinib on VEGFR2-HEK cells

FIG. 17.: Inhibitory effect of 5-defluoro-6-azido-vorolanib on HRMEC cells

FIG. 18.: Inhibitory effect of 5-defluoro-5-azido-6-chloro-vorolanib on HRMEC cells

FIG. 19.: Inhibitory effect of 5-defluoro-5-azido-sunitinib on HRMEC cells

FIG. 20.: Inhibitory effect of 5-defluoro-6-azido-sunitinib on HRMEC cells

FIG. 21.: Inhibitory effect of 6-azido-sunitinib on HRMEC cells

FIG. 22.: Inhibitory effect of 5-defluoro-5-azido-6-chloro-sunitinib on HRMEC cells

FIG. 23.: Inhibitory effect of 5-defluoro-5-azido-6-chloro-vorolanib on HRMEC cells

FIG. 24.: Network of HRMEC cells in the presence of 5-defluoro-5-azido-sunitinib with and without light

FIG. 25.: Network of HRMEC cells in the presence of 5-defluoro-6-azido-sunitinib with and without light

FIG. 26.: Network of HRMEC cells in the presence of 6-azido-sunitinib with and without light

FIG. 27.: Network of HRMEC cells in the presence of 5-defluoro-5-azido-6-chloro-sunitinib with and without light

FIG. 28.: Network of HRMEC cells in the presence of 5-defluoro-5-azido-6-chloro-vorolanib with and without light

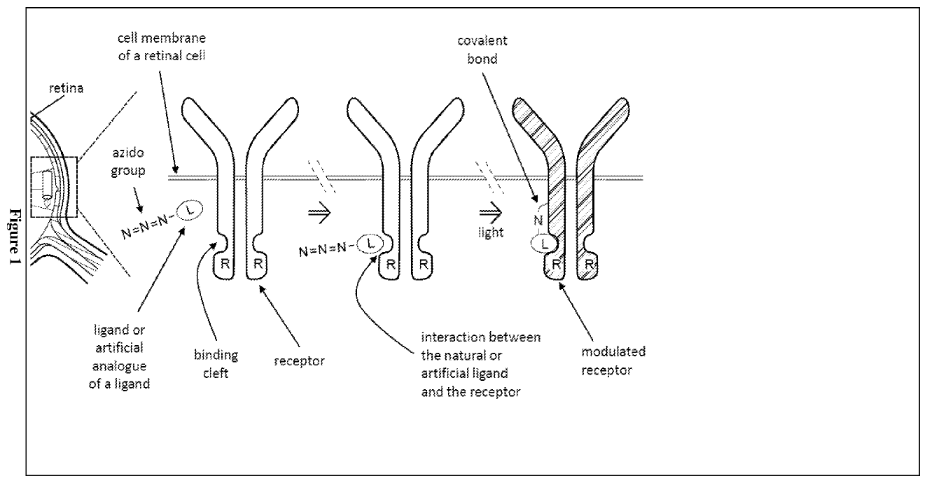

### DETAILED DESCRIPTION OF THE INVENTION

The idea of the present invention is related to a new way of targeting drug molecules (new chemical entities) to the tissues of the eye (especially the retina) that are reached by natural light seen by the patient or animal to be treated. The optical system of the eye consists of the cornea, the crystalline lens and the iris [Lombardo et al., 2013] and has evolved to focus the light onto the retina. The present invention capitalizes on the fact that through this mechanism the density of photons is higher within the retina than in any other internal, non-exposed tissues of a biological organism (animal or human being).

Using proof-of-concept experiments, the present inventors have found that azidation, a chemical intervention consisting of binding an azido group (N₃) to a chemical structure is a suitable modification to render the given parental molecule sensitive to light. Once the azidated molecule reaches the eye, in particular the retina, the molecule can undergo photoactivation by the ambient light seen by the patient to be treated. Light is known to convert azidated molecules into a reactive nitrene radical or a reactive cyclic ketene-imine (or equivalent reactive intermediate) that can then form a covalent bond with several functional groups of nearby biomolecules. Therefore, an azidated substrate or any azidated substrate analogue can be covalently linked to its cognate ocular binding partner, e.g. enzyme by natural light seen by the patient. Via the same mechanism, any azidated ligand or any azidated ligand analogue can be covalently linked to any cognate ocular receptor by natural light seen by the patient.

According to the concept of the invention, the covalent binding of the photoactivated azidated molecules gradually extracts such molecules from the circulation and enrich them in the eye, preferentially the retina, given the fact that from the plasma new, unactivated molecules can diffuse towards their retinal target, occupy their binding cleft on the target molecule and subsequently become photoactivated and covalently bound. Therefore, the application of the azidation is specifically contemplated in the present invention, especially the azidation of any chemical structure having the appropriate conjugated system that permits the natural in situ photoactivation (cleavage of an N₂ molecule from the azido group) in ocular tissues which are illuminated by ambient light (including but not limited to the retina) when its used with the aim of lowering the plasma concentration to avoid the unwanted side effects of a previously known therapeutic parental molecule that is otherwise effective against a particular ocular disease.

Vorolanib (X-82, CM082), an orally available VEGFR2 inhibitor, initially developed against tumor angiogenesis, was effective in a phase I clinical trial against neovascular AMD [Jackson et al., 2017]. Importantly, the subsequent phase II trial investigating whether the number of standard intravitreal anti-VEGF injections in neovascular AMD can be decreased upon oral vorolanib administration has been prematurely stopped, due to hepatobiliary and gastrointestinal adverse effects [Cohen et al., 2020]. Increasing specific interactions between the VEGFR2 (KDR) and its small molecule inhibitors (especially vorolanib) to replace intravitreal injections in the treatment of AMD and PDR patients would also allow reducing their dose and in turn their side effects. Therefore increasing the strength of the said interaction and turning it into a stable covalent bond addresses an unmet medical need.

The experiments presented herein demonstrate the capability of the azidated derivatives of vorolanib and sunitinib to inhibit VEGF triggered signaling via the VEGFR2 receptor. Sunitinib has been chosen on the basis that it is a well-described VEGFR2 inhibitor frequently used in anti-tumor therapy [Gan et al., 2009]. The present inventors have demonstrated in proof-of-concept experiments presented herein that irradiation of both the azidated sunitinib molecules and the azidated vorolanib molecules bound to their receptors strongly increases the inhibition that they exert on VEGFR2.

Currently the inhibition of VEGF signaling by intravitreal injection is the standard medical treatment of AMD and PDR. Per os taken azidated inhibitors of VEGFR2, as prototype molecules, have the potential to replace the conventional treatment of AMD and PDR. Therefore, the invention relates to the use of the azidation of any chemical structure in combination with the subsequent natural in situ photoactivation in ocular tissues (including but not limited to the retina) with the purpose of inhibiting VEGF signaling in such ocular tissues to medically treat patients suffering from AMD and PDR.

A high number of inhibitors of VEGF signaling, in particular small molecule VEGFR2 inhibitors are known in the art.

For illustration and by way of an example, Peng, [Peng, Fan-Wei'et al., 2017] and Farghaly [Farghaly, T A et al. 2021] report a high number of small-molecule inhibitors of and antibodies against VEGFR2 and their potential use as therapeutics against several types of cancers, angiogenesis-related disorders, and Parkinson's and Alzheimer's diseases in the patent literature of the period 2012 to the end of 2020.

Furthermore, Khanwelkar, Rahul R. et al. report on the synthesis and structure-activity relationship of 6-arylureido-3-pyrrol-2-ylmethylideneindolin-2-one derivatives as potent receptor tyrosine kinase inhibitors [Khanwelkar, R. R, 0.2010]. Yang T-H et al. [Yang T-H et al. 2017A and 2017B] and describe the synthesis and evaluation of novel 2-pyrrolidone-fused (2-oxoindolin-3-ylidene) methylpyrrole derivatives as potential multi-target tyrosine kinase receptor inhibitors.

From these and other publications it can be seen that there have been a number of VEGFR2 inhibitors present in the art which plausibly can be applied as an active agent moiety in the present invention, once azidated and optionally completed with a linker between the active agent moiety and the naturally photoactivable azide, in a plausible embodiment.

### Azidation

According to the invention azidation is a suitable chemical modification for in vivo retinal targeting of therapeutic molecules because it consists of the addition of minimal number of new atoms to a biologically active therapeutic molecule, therefore this modification has the smallest chance to interfere with the binding of the said therapeutic molecule to its biological target in the retinal tissue. The results of proof-of-concept experiments provided herein show that azidated versions of previously known substrate analogues (e.g. azidated versions of parental molecules) can inhibit their target biomolecule stronger in ambient light, e.g. in white light, than in dark.

The requirement of suitability for natural photoactivation within the eye imposes constraints onto the chemical structure of the azidated molecules that are to be used as drugs. Typical aryl-azide molecules can be photoactivated with light having a wavelength of 350 nm [Keana et al., 1990] that is not suitable for in situ photoactivation in the human retina by natural light since the lense of the human eye absorbs the light with wavelength under 400 nm [Kessel et al., 2010]. Furthermore, the transparency of the lense drastically decreases with an increasing age, and significant amount of light with wavelength over 450 nm is absorbed in the eye of people over 60 years before it could reach the retina [Kessel et al., 2010]. It has been shown that an increase of wavelength of maximal light absorption of aryl-azides also increases the efficiency of photoactivation [Keana et al., 1990]. Consequently, molecules having an absorption maximum of at least at 400 nm are suitable ones for azidation to obtain naturally photoactivable drug candidates. Therefore, in the prototype application presented in this document, the present inventors used biologically active molecules that can be azidated on a benzene ring that is part of a larger delocalized electron system (r-system) and correspondingly absorb light also at wavelengths higher than 400 nm. In each of the prototype molecule presented here, the azido group (N₃) was coupled to a carbon atom that is part of an extended conjugated 7-system so that the azido group can directly harness the energy of the light absorbed by such 7-system.

### Inhibition of Retinal VEGF Signaling

Given the extraordinary clinical importance of AMD and PDR, furthermore considering the fact that currently the anti-VEGF therapy is the preponderant, almost exclusive therapy of these conditions we have opted for the inhibition of retinal VEGF signaling as an exemplary application. Therefore, for the purpose of the present invention the exemplary molecules were chosen to be tyrosine-kinase inhibitors that can inhibit the activity of VEGFR2. It is demonstrated herein that the azidated versions of the said small molecule VEGFR2 inhibitors still bind to VEGF2 and inhibit its activity while azidation renders them photoactivable. As a result of the photoactivation, the specific inhibitory potential of these molecules increases.

### Exemplary Scaffolds to Prepare Photoactivable Azido-Compounds

Sunitinib (3), vorolanib (4) and famitinib (5) are all small molecule tyrosine kinase inhibitors capable of exerting inhibitory effect on VEGFR2 receptor and they all share a common structural element consisting of a 3-[(1H-pyrrol-2-yl)methylidene]-1,3-dihydro-2H-indol-2-one moiety (hereinafter referred to as “PMO”, as an abbreviation for the simplified name “pyrrol-methylidene-oxindole” for the purpose of the present patent, see general formula (I) wherein the substituents are defined in the Brief Description of the invention as well as in the appended claims).

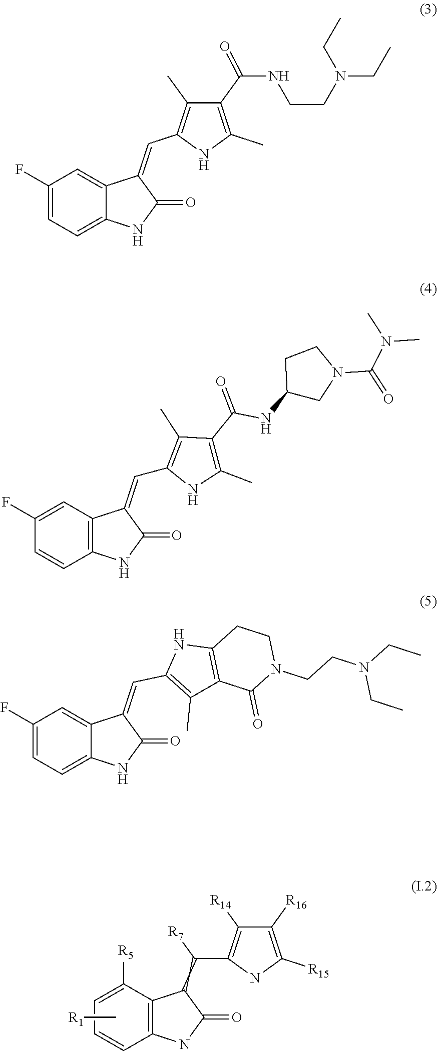

The PMO is on one hand side necessary for the binding of these three molecules to the VEGFR2 receptor and contains on the other hand a large delocalized electron system (7-system). The PMO moiety is present in an unchanged form in sunitinib, vorolanib and famitinib, is responsible for the absorption of visible light, and confers a yellow color to these molecules. We therefore decided to place an azido group onto the benzene ring of the PMO to achieve a wavelength of photoactivation higher than 400 nm. This wavelength is required based on the spectral properties of the natural light that can penetrate into the human eye via the cornea and the lens [Kessel et al., 2010]. The exemplary prototype molecules presented here are based either on sunitinib or on vorolanib. For all of the prototype molecules presented here, the azido group (N₃) was placed in different positions of benzene ring. Correspondingly, in some of the said prototype molecules, the fluorine atom (F) was omitted.

In further exemplary prototype molecules both the fluorine atom and the azido group was present, or, instead of fluorine, other halogen substituents like chlorine atom and bromine atom have been used, as shown in the Examples, see formulae (6) to (8), wherein the azido group is in the 5 while the halogen atom is in the 6 position. In other examples the 5 and 6 positions of the azide and the other substituent may be the opposite (5-halogen 6-azide derivatives).

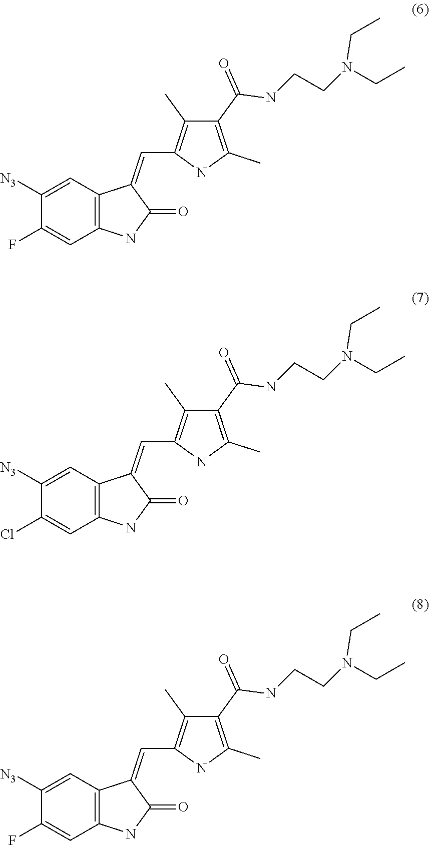 [SMILES: CCN(CC)CCNC(=O)c1c(C)[nH]c(/C=C2\C(=O)Nc3cc(F)c(N=[N+]=[N-])cc32)c1C ; CCN(CC)CCNC(=O)c1c(C)[nH]c(/C=C2\C(=O)Nc3cc(Cl)c(N=[N+]=[N-])cc32)c1C ; CCN(CC)CCNC(=O)c1c(C)[nH]c(/C=C2\C(=O)Nc3cc(F)c(N=[N+]=[N-])cc32)c1C]

Formula (I.2) illustrates this concept of the invention by the example of the scaffold of sunitinib, vorolanib and famitinib and is derivable therefrom e.g. by substitution of F by an azido group R₁. It is understood that the newly introduced azido group (R₁) can be in any position of the aryl ring of the oxindole moiety, as shown by formula I.2. Further groups R₇ and R₁₄, R₁₅ and R₁₆ are as defined herein, particularly in the Brief description of the invention or in the appended Claims. It is also known by a person skilled in the art that the double bond adjacent to the oxindole moiety can be either cis or trans position as shown in formula (I.2)

However, it is known and within the skills of a person skilled in the art to use a compound wherein the aryl ring of the oxindole moiety is further substituted and the double bond adjacent to the oxindole moiety can be either cis or trans position, for example as shown in formula (I.1):

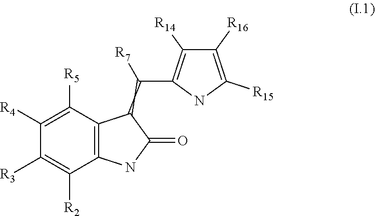

wherein at least one of substituents R₂, R₃, R₄, R₅ is an azide and the other substituents, as well as R₇ and R₁₄, R₁₅, and R₁₆ are as defined herein, particularly in the Brief description of the invention or in the appended Claims.

To demonstrate the inhibitory effect of the prototype molecules, the inventors have conducted experiments in a biological system designed to report the strength of VEGF signaling. Inventors have used human embryonic kidney (HEK cells) harboring two exogenous genetic constructs, the first one expressing the VEGFR2 targeted to the cell surface, the second one encoding the luciferase gene under the control of a specific promoter (NFAT promoter) that is responsive to the intracellular signaling events triggered by the receptor. This cell culture based system was validated by adding exogenous VEGF with and without well-known previously characterized inhibitors of VEGFR2 and the validation provided proof that the light generated by the luciferase enzyme is suitable to measure the strength of VEGF signaling. Using the validated cell-culture based system, it has been demonstrated that the azidated prototype molecules can bind to their target similarly as their previously known parental molecules. By comparing the results of experiments conducted in the dark and those conducted in illuminated conditions, it has also been demonstrated that light can photoactivate the prototype molecules and can potentiate their biological effect.

To further demonstrate the inhibitory effect of the prototype molecules, the inventors have conducted experiments using commercially available human retinal microvascular endothelial cells (hereinafter referred to as “HRMEC”) isolated post mortem from human donors. These cells preserve their endothelial nature, and when cultured on an appropriate extracellular matrix, they spontaneously form a network that corresponds to a network of capillaries. Images of such networks taken with a microscope can be quantified, and several parameters (such as the number of segments, number of closed loops, number of junction points) of the network can be quantified and used as a parameter describing the angiogenesis in vitro [Staton et al., 2009].

The inventors have applied their proprietary azidated inhibitors described in the present patent in an ascending series of concentrations. For all the said azidated inhibitors, the in vitro angiogenesis experiments were carried out both in complete darkness and in defined illumination conditions. The number of closed loops (number of meshes) was identified using an image recognition algorithm, and an IC50 curve was fitted onto the number of meshes plotted against substance concentration. The inventors have found that for several candidate molecules presented here, the IC50 values in the light significantly differed from the IC50 values in the dark. These experiments thus provided further proof, that light increases the inhibitory potential of the novel azidated compounds described in the present patent. Taken together, the experiments presented here demonstrate that azidated small molecule inhibitors can bind to their cognate target, and the newly added azido groups do not prohibit the receptor-inhibitor interaction and do not abrogate the functional consequences of such interaction. Furthermore, the experiments presented here also demonstrate that small molecule inhibitors that are transiently, non-covalently bound to their targets are photoactivated by light and such photoactivation can trigger a covalent binding to the biological target which the molecules are already associated to. Finally, the experiments presented here also demonstrate that light-triggered covalent binding between the receptor and the small molecule inhibitor has functional consequences and enhances the inhibitory potential of the azidated small molecules.

### Concept of Prodrugs

The compounds of the invention may be administered in the form of prodrugs. The concept of prodrugs involves the derivatization of a functional group of the compound that renders the molecule inactive, whereas the prodrug is converted, via metabolic processes to the useful active agent in vivo, e.g. in the patient. Typically, the derivatized functional group is metabolized to the original structure.

As a preferred example, in a preferred embodiment, a —CH₂—OH moiety is attached to the nitrogen atom of the indol-2-one structure, and a suitable further moiety that is recognized by an active transporter expressed in the ocular target cells is attached via forming an ester bond with the said —CH₂—OH. Such modification of the molecules offers the advantage of (i) actively taken up by transporter molecules expressed in the cells of the eye and (ii) hidrolyzed by estherase enzymes once the molecule is in the intracellular compartment, this way the molecule becomes active. Examples of such products include those described by Wang et al. [Wang et al., EP3252048A4] and by Buchy E., et al. [Buchy E., et al. 2015].

### Preparation of Azido Compounds

Various methods for the synthesis of aryl-azides are well known to a person skilled in the art.

Without providing a full review we provide the following summary and refer to the following exemplary methods herein.

Aryl azides are traditionally prepared by treatment of diazonium salts with an azide anion, however, several other methods exist. Typically nucleophilic displacement by an azide anion can be accomplished only if the aromatic ring is activated. Mild conditions are reported in the literature to avoid e.g. nitrogen loss or decomposition under harsh conditions [D'Anna et al. 2008]

Aromatic azides can be prepared by various methods including substitution of halogens in activated aryls by the azide anion; interaction of azides (e.g. Me₃Si—N₃, Tos-N₃ or NaN₃,) with organometallic aryl reagent (eg. Grignard or aryllithium reagents); diazotization of aryl hydrazines or by reacting aromatic amines with TfN₃ or other reagents, using hydrogen azide reagent on nitrosoarenes; base induced decomposition of triazenes. Direct methods of introduction of azides to arenes also exist and rely on using NaICl₂ and NaN₃ [Griganov et al. 2016].

A relatively general method is to prepare aryl-azides from aryl-halides. As a pseudohalide, azide readily displaces many leaving groups, e.g. Br⁻, I⁻, sulfonate, and others to yield the azido compound. The azide source is most often sodium azide (NaN₃), although lithium azide (LiN₃) is also applicable.

As an example, Andersen, J. et al describe a rapid synthesis of aryl-azides from the corresponding aryl-halides catalyzed by CuI/diamine using sodium ascorbate as a stabilizer of the catalyst system under very mild conditions generally with high yields. [Andersen, J. et al. 2005]

Alternatively, Hajipour A. R. et al. have developed a method via the reaction of aryl-halides with sodium azide under Cu₂O/tetraethylammonium prolinate catalysis. [Hajipour et al. 2014]

Preferred compounds of the invention depicted in Formula I.1. can be synthesized according to the following Scheme 1:

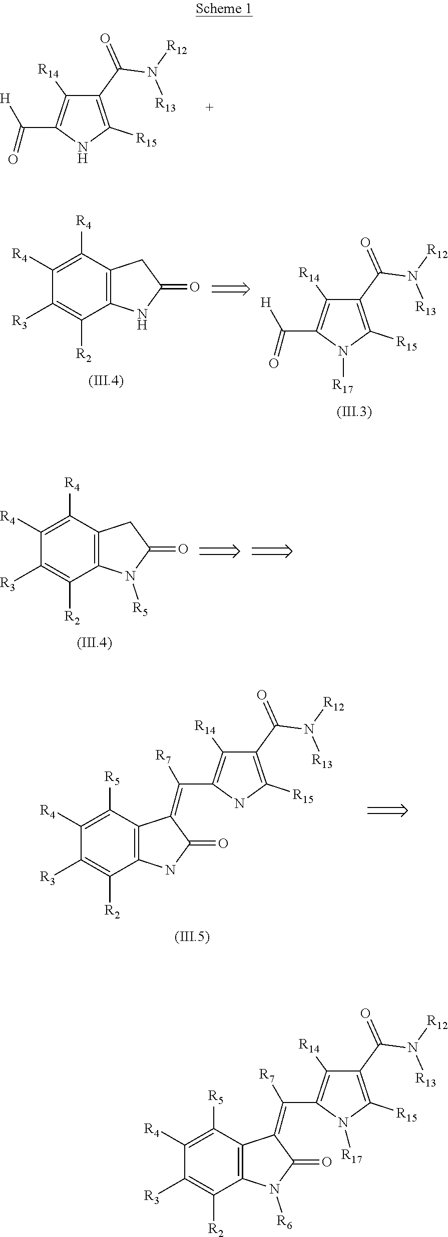

The key intermediate (III.3) for preparation of target compounds can be synthesized according to the method as described in Yang et al. 2017.

Compound (III.3) can be dissolved e.g. in EtOH and a solution of the oxindole derivative carrying the azido group is added in the same solvent e.g. in EtOH in stochiometric amount. The reaction is carried out in the presence of minor amount of piperidine. After stirring, the compound is obtained as a precipitate, which is filtrated, washed and purified. The yield is typically between 40 to 70%. The reaction must be carried out with continuous protection from light to avoid activation of the azide.

In an embodiment the synthesis of (III.3) may start from compound (III.1) (2-tert-butyl 4-ethyl 3,5-R₁₄,R₁₅-1H-pyrrole-2,4-dicarboxylate),

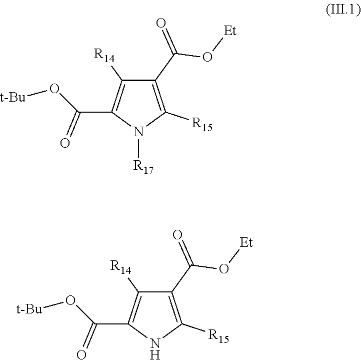

which can be prepared via the Paal-Knorr pyrrole synthesis [Kennedy et al. 2009]. R₁₅ and R₁₄ may be functional group(s). Provided that R₁₅ and R₁₄ are alkyl, e.g. methyl, formyl functionality can be provided by selective oxidation (e.g. with ceric ammonium nitrate (CAN) at room temperature).

In case R₁₅ and R₁₃ should not form a ring the OEt of the 4 carboxylate can be converted into corresponding amide in any known way for amidation to provide —C(O)NR₁₂,R₁₃. Converting esters into amides are well known in the art, see e.g. [Montalbetti et al., 2005; Valeur et al., 2009; Millera et al, 2015 and references cited therein].

In case R₁₅ and R₁₃ should form a ring, for example, a carboxyl (e.g. a formyl) functionality is to be provided, which is then converted into a secondary amine which in turn forms the amide with the 4 ester of the pyrrole ring as described in [Yang et al 2017A; Yang et al. 2017B].

The azidated oxindole compounds to be coupled with the aldehyde (III.3.) can be prepared e.g. via a oxindole derivative substituted with halogenide (e.g. I⁻) wherein said halogenide is subsequently replaced by azide, given the fact that azide is a pseudohalogenide. The oxindole derivative substituted with halogenide is prepared—in one particular embodiment—from 3-iodoaniline or 4-iodoaniline (to be decided on the basis of where the azido group in the final product is intended to be), then preparing the (2E)-N-(4-iodo)-2-(hydroxiamino)acetamide by a condensation reaction, subjecting the product to ring closure to obtain the appropriate isatin derivative, and finally reducing the isatin to produce the oxidole-derivative containing an iodine atom in the required position.

It is known by a skilled person that alternative methods can also be applied.

### Pharmaceutical Compositions

In the present invention the compounds used are typically rather hydrophobic, light sensitive compounds. Thus, when formulating them into pharmaceutical compositions these problems should be considered.

Poor solubility resulting in limited drug loading can occur as a problem which has to be solved. To increase the solubility, cyclodextrins can be used.

(In case of sunitinib, marketed under the trade name Sutent, a malate salt is applied.)

Typical excipients may include the following categories and examples:

Disintegrants, like crosslinked polymers, polyvinylpyrrolidone (crospovidone), crosslinked sodium carboxymethyl cellulose (croscarmellose sodium), in particular the latter.

Binders, including for example
saccharides e.g. disaccharides (lactose, saccharose); polysaccharides (e.g. cellulose, starches etc.), modified polysaccharides such as microcrystalline cellulose, cellulose ethers etc.;
sugar alcohols such as xylitol, sorbitol or mannitol; in particular mannitol (E421)
protein type binder, like gelatin; (in particular in light gelatin capsule).

Lubricants, like magnesium stearate, or other stearate derivative (or talc or silica etc.).

Polymers having the role of a stabilizer, surfactant thickening agent, solubility enhancer, e.g. Povidone (polyvinylpyrrolidone, PVP) or other synthetic polymers, like polyethylene glycol (PEG), preferably Povidone.

A preferred formulation is capsule, e.g. light gelatin capsule or hard capsule wherein the active agent is protected from light. In an embodiment the package should protect the azidated compounds from light.

Non-transparent capsules are preferred for oral administration, the said capsules are to be designed with optical properties suitable to protect the azidated molecules from photoactivation.

An example is Sutent gelatine capsules comprising Gelatin, red iron oxide (CI 77491) (E172) and titanium dioxide (CI 77891) (E171) arranged in a way to protect the active agent from light.

Another example is Lonza Capsugel of TiO₂-free light-protected capsules (See [Lonza Press Release “Lonza Expands its Capsugel® Capsule Offering to Include Titanium Dioxide-Free White Hard Gelatin Capsules” May 9, 2022, Basel, Switzerland]).

Methods for encapsulation or incorporation into polymeric matrices, including nano- and micro-particles, with increased loading are also known, see e.g. WO2016100392A1 [Fu J. et al., 2016]. and related compound is disclosed as a self-nanoemulsifying formulation [Nazari-Vanani et al., 2017].

In a further embodiment, the formulation is an eyedrop. In this case, given the hydrophobic nature of the PMO moiety present in all the compounds, a solubilizing agents, such as encapsulation methods, (e.g. by cyclodextrines) or vesicular systems (e.g. liposomes) are needed. These methods are reviewed by Ioele et al. [Ioele et al., 2017]. Further encapsulation methods may be provided by microparticles surrounded by a coating material. Lipid nanoparticles or polymeric nanoparticles are also useful technologies in providing light protection. Alternative methods may be provided by nanoemulsions which may be oil-in-water (O/W) or water-in-oil (W/O) emulsions. Several of these methods are reviewed by Coelho L et al [Coelho L et al., 2018].

These formulations can also be made photoprotective which is of key importance for the photolabile azidated molecules described in the present patent.

The present invention is further illustrated by way of non-limiting examples.

### EXAMPLES

### Azido Derivatives of PMO-Containing Molecules

### A) Sunitinib-Based Compounds

### Preparation of Azide-Substituted Sunitinib Derivatives of the Present Invention:

First, iodine-substituted indol-2-ones are synthesized starting from para-iodo-aniline and meta-iodo-aniline carrying the appropriate substituents, by the corresponding amidation with reaction to form (2E)-N-(4-iodo)-2-(hydroxyimino)acetamide and (2E)-N-(3-iodo)-2-(hydroxyimino)acetamide, respectively, from which the respective product is obtained by ring closure.

Subsequently, to prepare the portion of the molecule that is to be coupled to the 3^{rd} carbon atom of the appropriately substituted indole-2-one structure, 2-tert-butyl 4-ethyl 3,5-dimethyl-1H-pyrrole-2,4-dicarboxylate (6) is synthesized via the Paal-Knorr pyrrole synthesis [Kennedy et al. 2009]. The 4-ethyl-carboxylate group is amidated with amino-trietylamine (8) in the presence of group (IV) metal alkoxide complexes or lanthanum trifluoromethanesulfonate or lithium-hydroxide as catalists [Millera et al., 2015 and references cited therein].

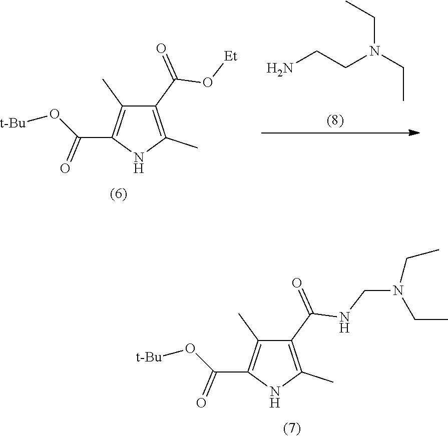

To each stirred solution containing compound 7 in EtOH is added, dropwise, a solution of appropriately substituted indole-2-one and then piperidine. After stirring at room temperature for 6 h, the precipitate formed is filtrated, washed with EtOH, and purified by column chromatography (silica gel, 90:10:1 EtOAc-MeOH-TEA) to form the required iodinated sunitinib intermedier.

Finally, the azido functionality is built up by replacing iodine on the sunitinib scaffold by using CuI/diamine catalyst, sodium ascorbate as a stabilizer of the catalyst and 2 equivalents of NaN₃ in a 3:7 medium of EtOH and H₂O [Andersen, J. et al. 2005].

The formulae of exemplary molecules having sunitinib scaffold that we have used for the experiments described in this patent are shown below as formulae (1) to (6), respectively.

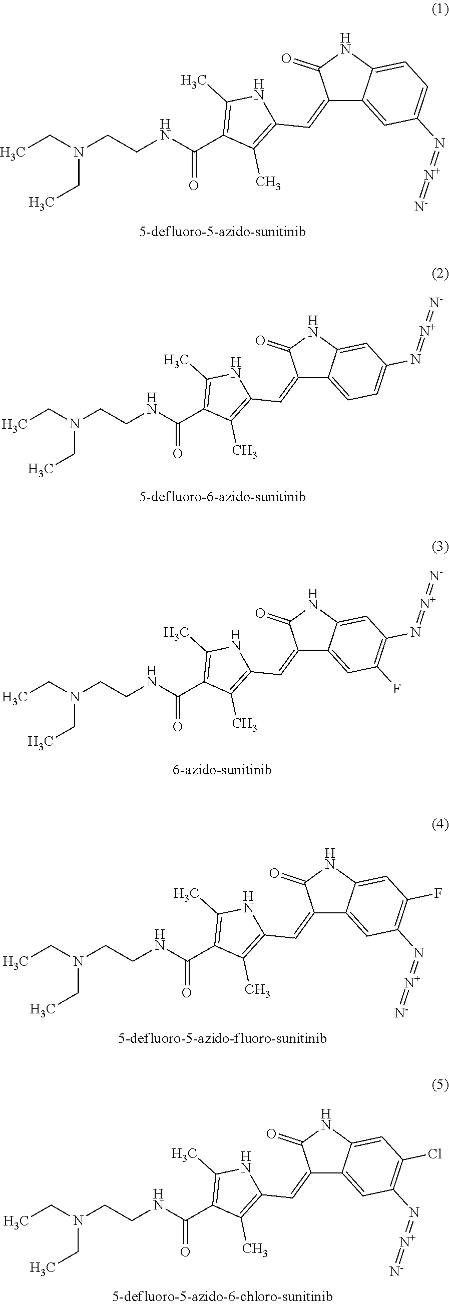 [SMILES: CCN(CC)CCNC(=O)c1c(C)[nH]c(/C=C2\C(=O)Nc3ccc(N=[N+]=[N-])cc32)c1C ; CCN(CC)CCNC(=O)c1c(C)[nH]c(/C=C2\C(=O)Nc3cc(N=[N+]=[N-])ccc32)c1C ; CCN(CC)CCNC(=O)c1c(C)[nH]c(/C=C2\C(=O)Nc3cc(N=[N+]=[N-])c(F)cc32)c1C ; CCN(CC)CCNC(=O)c1c(C)[nH]c(/C=C2\C(=O)Nc3cc(F)c(N=[N+]=[N-])cc32)c1C ; CCN(CC)CCNC(=O)c1c(C)[nH]c(/C=C2\C(=O)Nc3cc(Cl)c(N=[N+]=[N-])cc32)c1C]

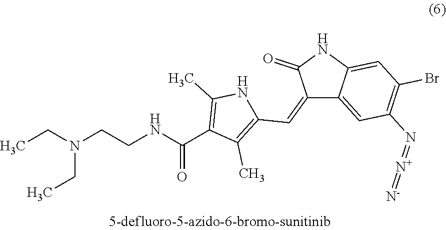 [SMILES: CCN(CC)CCNC(=O)c1c(C)[nH]c(/C=C2\C(=O)Nc3cc(Br)c(N=[N+]=[N-])cc32)c1C]

In the sunitinib-based molecules presented above, the covalently attached azido group (N₃) becomes part of an extended conjugated π electron system.

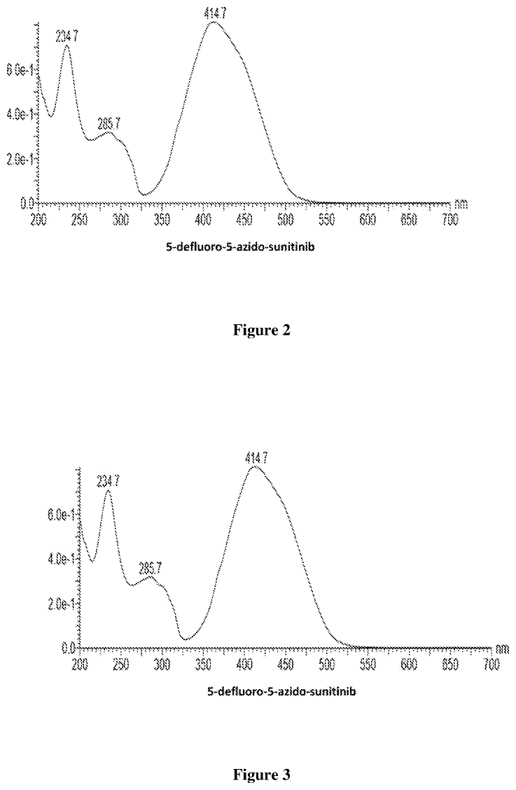

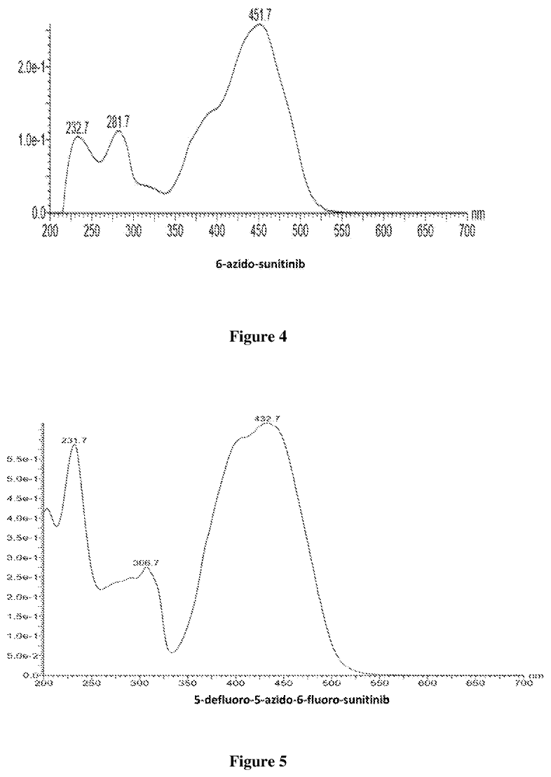

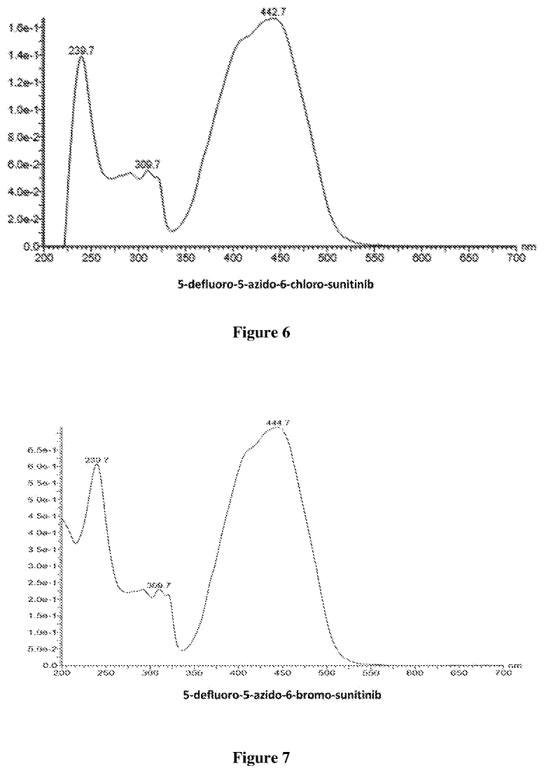

Sunitinib has an absorbance maximum at 430-431 nm which can be shifted even higher in the azidated versions as shown by FIGS. 2-7.

### B) Vorolanib-Based Compounds

### Preparation of Azide-Substituted Vorolanib Derivatives of the Present Invention:

The indole-2-one moiety of these molecules were prepared in an identical manner as described for the molecules based on the sunitinib scaffold. Subsequently 2-acetaldehydo-4-carboxy-3,5-dimethyl-1H-pyrrole is coupled to the appropriate indole-2-one in ethanol, and in the presence of hexafluorophosphate-azabenzotriazole-tetramethyl-uranium (HATU), N,N-diisopropylethylamine (DIPEA) and dimethylformamide (DMF) the required amide [Carpino, 1993] is formed using N′-pyrrolidino-N,N-dimethyl-urea as the cyclic amine to build up the vorolanib scaffold. Finally, the azido functionality is introduced into the molecule by replacing iodine on the indole-2-one moiety of the vorolanib scaffold using CuI/diamine catalyst, sodium ascorbate as a stabilizer of the catalyst and 2 equivalents of NaN₃ in a 3:7 medium of EtOH and H₂O [Andersen, J. et al. 2005].

The formulae of exemplary molecules having vorolanib scaffold that we have used for the experiments described in this patent are shown below as formulae (7) and (8), respectively.

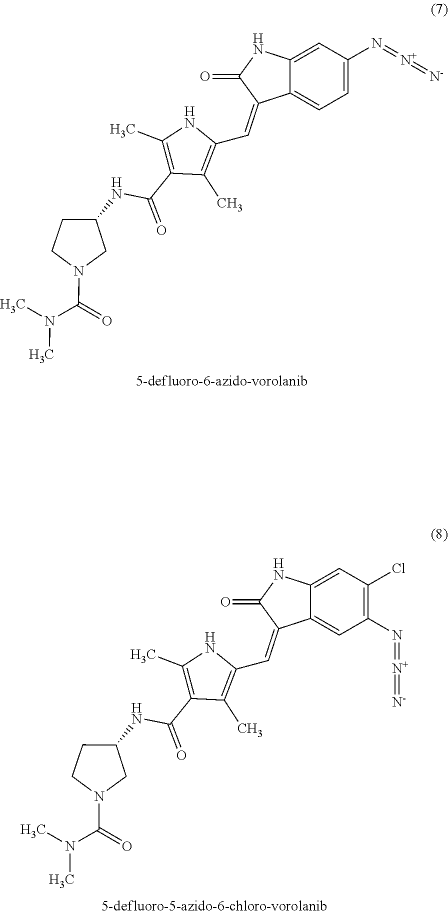 [SMILES: Cc1[nH]c(/C=C2\C(=O)Nc3cc(N=[N+]=[N-])ccc32)c(C)c1C(=O)N[C@H]1CCN(C(=O)N(C)C)C1 ; Cc1[nH]c(/C=C2\C(=O)Nc3cc(Cl)c(N=[N+]=[N-])cc32)c(C)c1C(=O)N[C@H]1CCN(C(=O)N(C)C)C1]

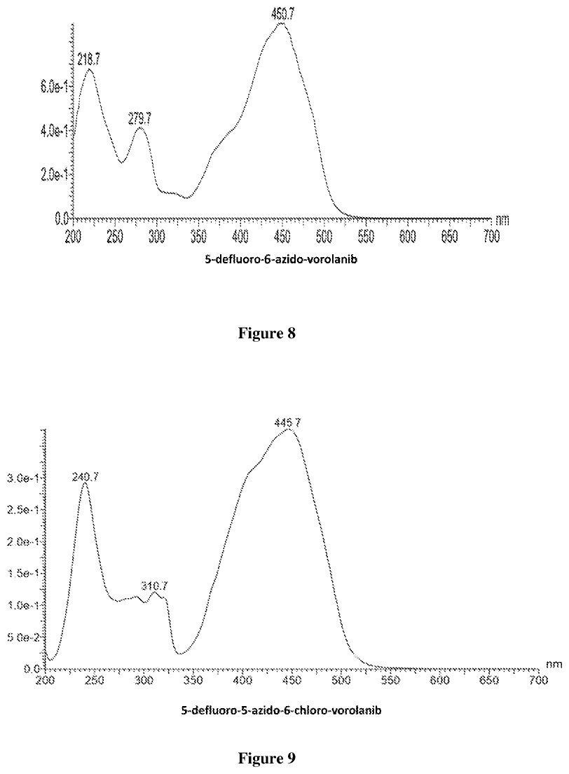

As evident for a person skilled in the art from the formulae above, the covalently attached azido group (N₃) becomes part of an extended conjugated π electron system, therefore the energy of the visible light harvested by the PMO of vorolanib can be transferred to the azido group to propel N₂ extrusion. The UV-visible spectra of the prepared vorolanib-based molecules above are shown in FIGS. 8.-9.

### Azidated Versions of PMO-Containing Molecules are Capable of Specifically Inhibiting VEGFR2 in a Light-Potentiated Way

The following experiments demonstrated that the azidated versions of sunitinib and vorolanib could bind to the VEGFR2 (KDR) receptor, inhibit its function, and light could potentiate this inhibitory effect.

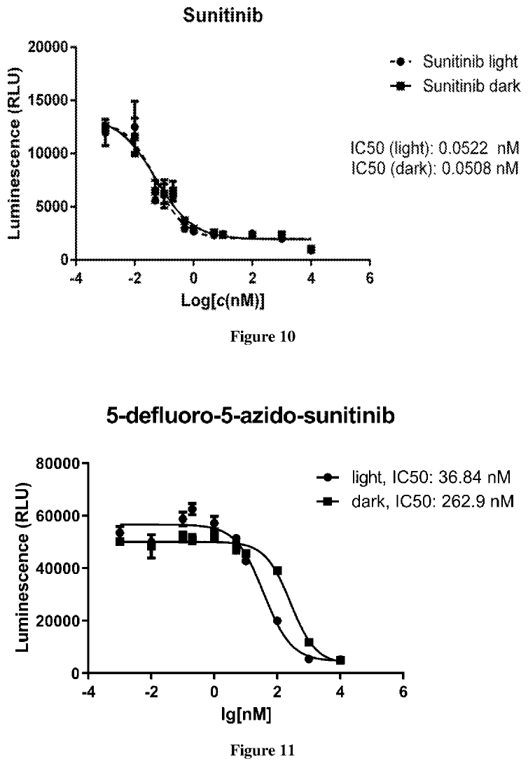

FIG. 10 shows the inhibitory effect of sunitinib in the presence of ambient light and without light, in the dark. Luminescence is plotted as a function of the logarithm of concentration in the light or in the dark whereby IC50 (i.e. concentrations at which the inhibition is half of the maximal inhibition) values can be calculated. Essentially the two curves run together and the IC50 values are nearly identical. Thus, binding properties and inhibition are independent of the ambient light.

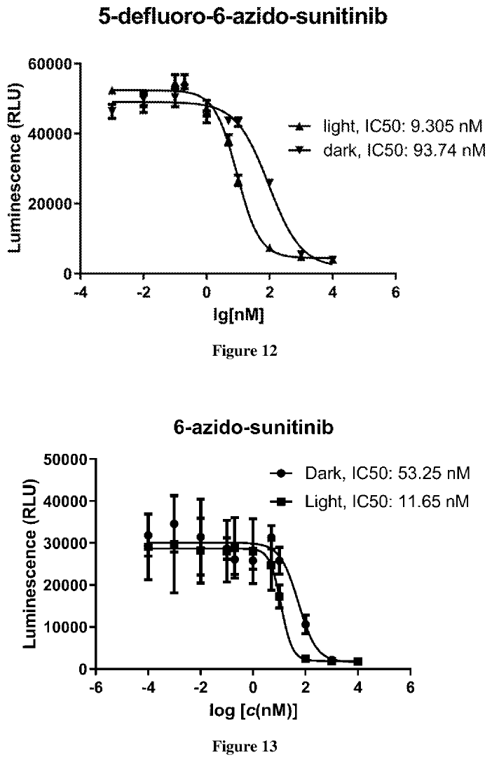

In FIG. 11 the inhibitory effect of 5-defluoro-5-azido-sunitinib is shown in the presence of light and in the absence of light. Ambient light induced photoactivation of this molecule is clearly seen on the plot. Upon illumination, the IC50 value for 5-defluoro-5-azido-sunitinib is nearly an order of magnitude lower than in the dark. The IC50 (light) is 36.84 nM whereas IC50 in the dark is 262.9 nM. The effect of the light is even more pronounced in the case of 5-defluoro-6-azido-sunitinib (FIG. 12) where the azido group occupies a different position of the indole-2-one scaffold, as evidenced by the fact that the IC50 value is more than ten times lower in the light (9.305 nM) than in the dark (93.74 nM).

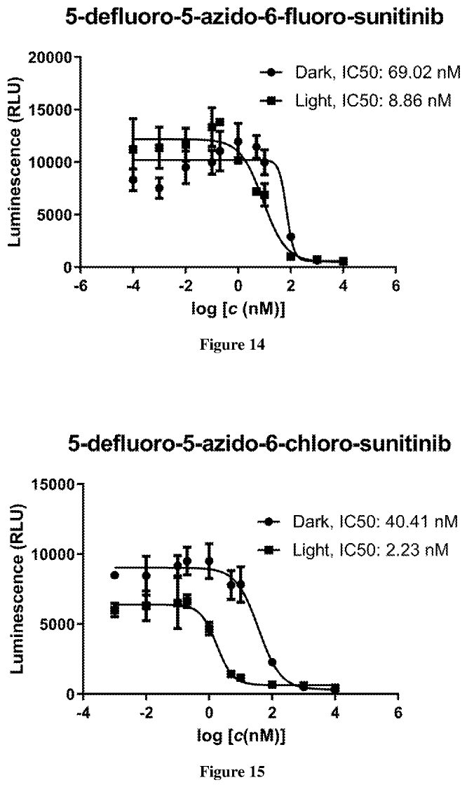

To study the effects of halogen atoms attached to the azido-indole-2-one moiety, 2 fluorine-containing variants have been synthetized: (i) sunitinib azidated in the 6^{th} position of the indol-2-one moiety displaying an IC50 of 53.25 nM (dark) and of 11.65 nM (light) as shown in FIG. 13, and (ii) the same molecule but with the fluorine atom and the azido-group swapped displaying an IC50 of 69.02 nM (dark) and of 8.86 nM (light), as shown in FIG. 14.

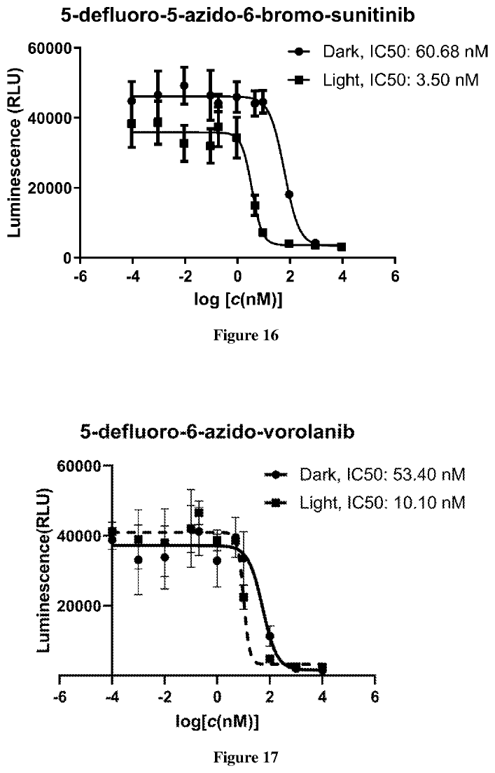

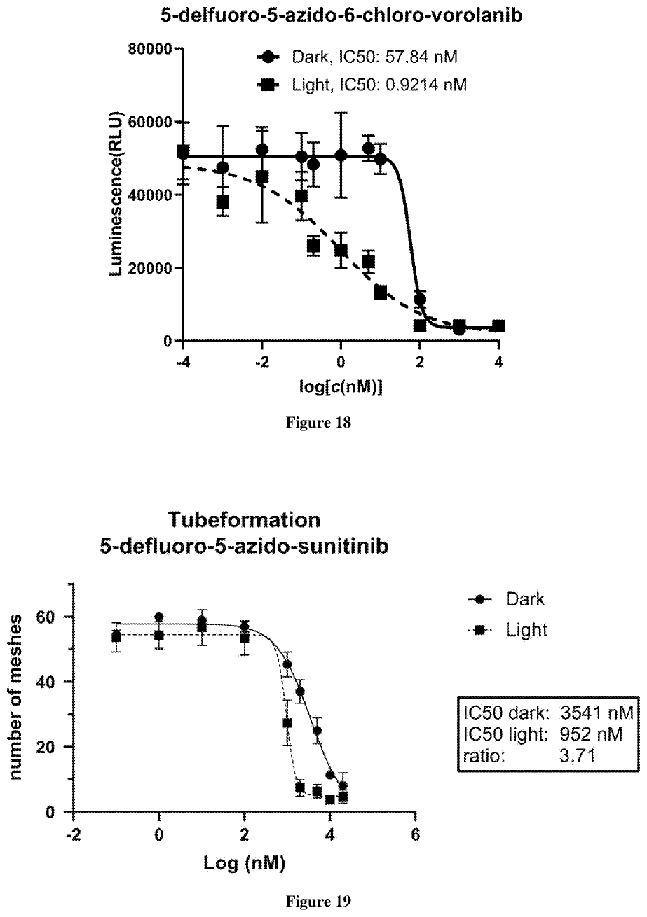

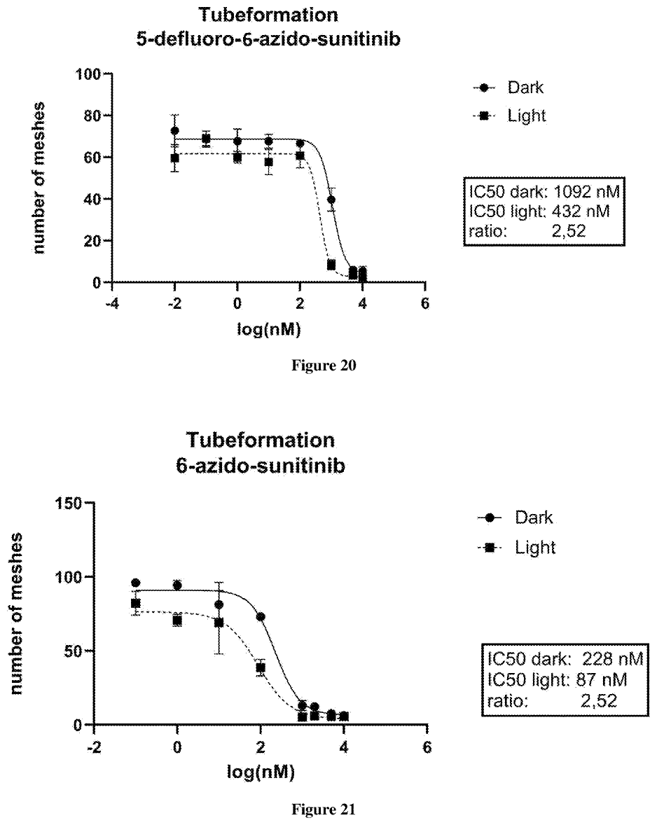

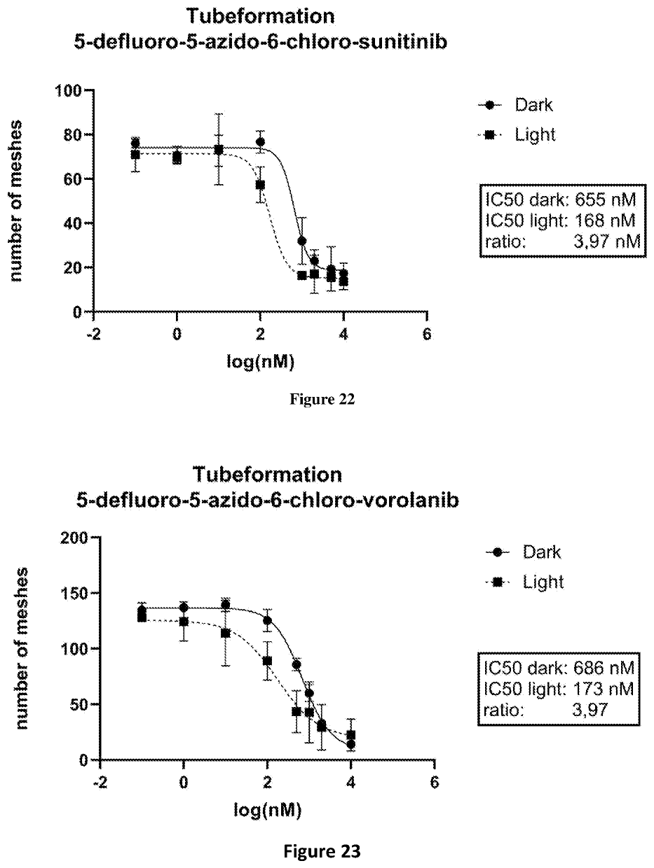

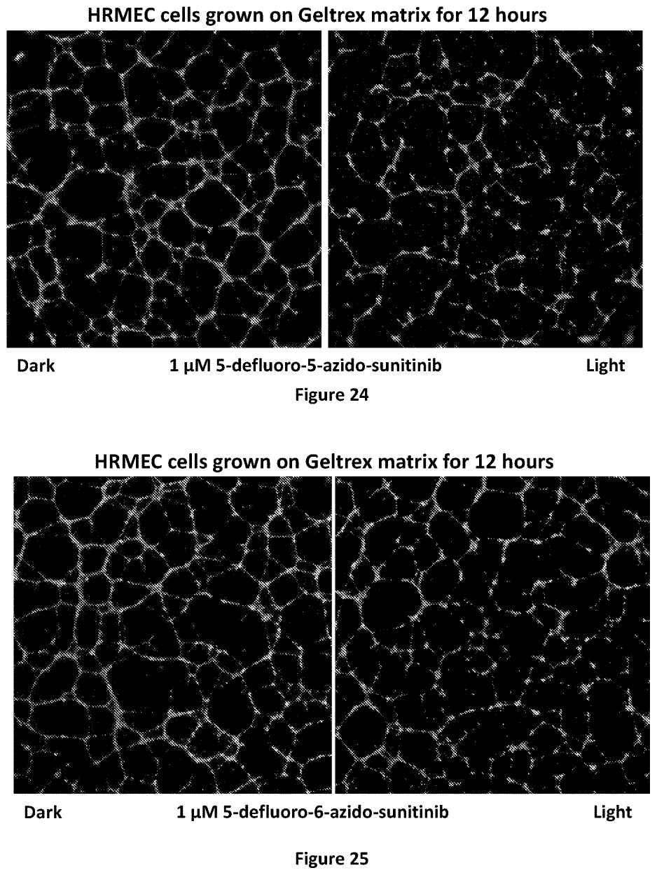

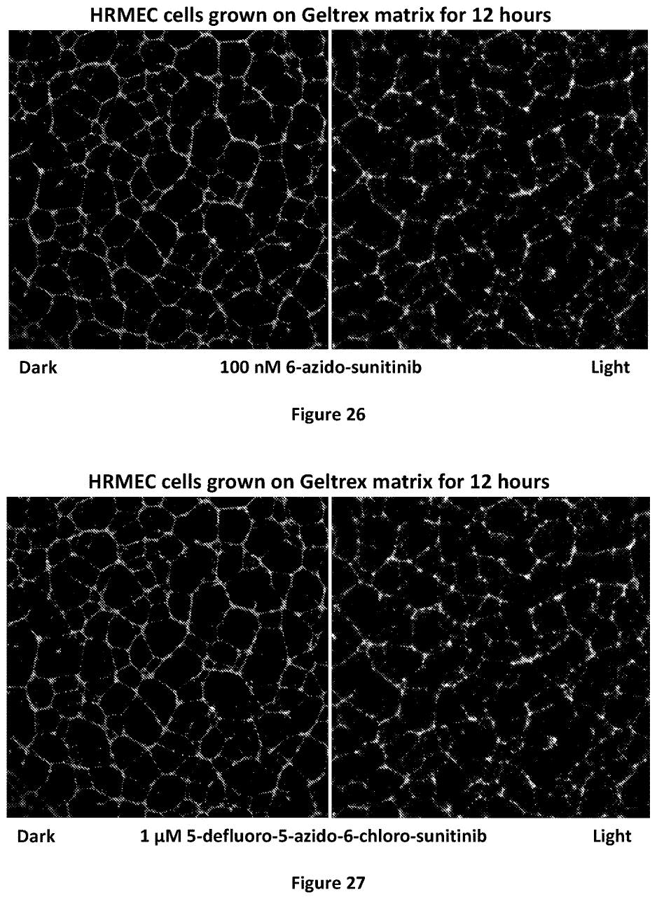

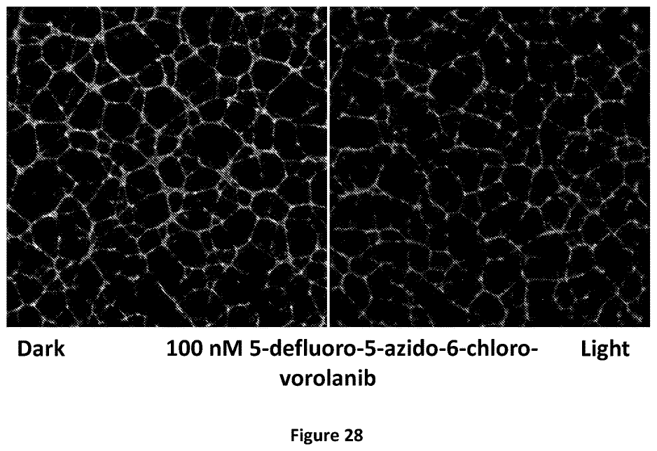

Using halogen atoms of larger molecular weight is supposed (i) to change the polarity of the structure via an inductive effect thereby improving the penetration into the cells and (ii) to change the π-electron density of the indole-2-one moiety via the mesomeric effect. Therefore the inventors have synthetized two derivatives with heavier halogen atoms: (i) 5-defluoro-5-azido-6-chloro-sunitinib displaying an IC50 of 40.41 nM (dark) and 2.23 nM (light) as shown in FIG. 15 and (ii) 5-defluoro-5-azido-6-bromo-sunitinib displaying an IC50 of 60.38 nM (dark) and 3.50 nM (light) as shown in FIG. 16. Taken together it can be concluded that chlorine and bromine atoms greatly enhanced the responsiveness of the molecules to light and increased their suitability for the purpose described in the present patent.

Importantly, in addition to sunitinib, a further scaffold, namely vorolanib could be successfully modified to include a photoactivable azido group. By removing the fluorine atom from the 5^{th} position of the indole-2-one moiety, and introducing an azido group into the 6^{th} position, the inventors obtained a molecule having an IC50 of 53.4 nM in the dark and 10.1 nM when illuminated with a cold white LED lamp (Table 1, FIG. 17.). Of note, orally administered vorolanib has already been investigated in human clinical trials against age-related macular degeneration [Jackson et al., 2017, Cohen et al., 2020] that were prematurely stopped in spite of the promising therapeutic efficacy, due to the intolerable levels of side effects. Therefore, modification of vorolanib according to the invention described herein would particularly be useful to lower the concentration of the medication in the plasma of the patients and thereby reducing the unwanted effects. Motivated by this fact, the inventors have introduced a chlorine substituent into the 6^{th} position of the oxindole moiety of vorolanib, given that the congruent modification proved to enhance the light-mediated activation of sunitinib. This way the inventors obtained 5-defluoro-5-azido-6-chloro-vorolanib the IC50 of which was 57.84 nM in the dark and 0.921 nM in the light (Table 1, FIG. 18.) displaying therefore a 62.80-fold light effect which qualifies this molecule to be the most efficient example among the compounds listed in Table 1.

Summary of IC50 values in VEGFR2 reporter HEK cell based assay system is shown in Table 1.

TABLE 1
IC50 values measured on VEGFR2-HEK cells

| Inhibitor (number of formula) | FIG. | Dark | Light | Dark/Light |
|---|---|---|---|---|
| sunitinib | 10 | 0.05082 nM | 0.05218 nM | 0.974 |
| 5-defluoro-5-azido-sunitinib (1) | 11 | 262.9 nM | 36.84 nM | 7.136 |
| 5-defluoro-6-azido-sunitinib (2) | 12 | 93.74 nM | 9.305 nM | 10.074 |
| 6-azido-sunitinib (3) | 13 | 53.25 nM | 11.65 nM | 4.57 |
| 5-defluoro-5-azido-6-fluoro-sunitinib (4) | 14 | 69.02 nM | 8.86 nM | 7.79 |
| 5-defluoro-5-azido-6-chloro-sunitinib (5) | 15 | 40.41 nM | 2.23 nM | 18.12 |
| 5-defluoro-5-azido-6-bromo-sunitinib (6) | 16 | 60.68 nM | 3.50 nM | 17.25 |
| 5-defluoro-6-azido-vorolanib (7) | 17 | 53.40 nM | 10.10 nM | 5.29 |
| 5-defluoro-5-azido-6-chloro-vorolanib (8) | 18 | 57.84 nM | 0.921 nM | 62.80 |

### Azidated Versions of PMO-Containing Molecules Inhibit In Vitro Angiogenesis and Such Inhibition is Potentiated by Light

In vitro angiogenesis (hereinafter referred to as “tubulogenesis”) mimicks several features of angiogenetic processes observed in vivo in vertebrate animals [Staton et al., 2009] and is based on the VEGF-dependent self-organizing capacity of endothelial cells via which tube-like structures emerge that share many properties with in vivo observed blood vessels. Therefore, the inhibitory potential of the newly synthetized molecules of the present invention was also assessed in tubulogenesis assay using human retinal microvascular endothelial cells (HRMEC) derived from post mortem human donors. When seeded onto a collagen and laminin containing matrix, HRMEC spontaneously form a two-dimensional blood-vessel-like network displaying several quantifiable features that can be used to measure angiogenic potential [Staton et al., 2009]. The newly synthetized, PMO-containing VEGFR inhibitors were added to the culture medium in increasing concentrations to test whether they can inhibit the spontaneous formation of the network from HRMEC. The increasing concentrations allowed the inventors to calculate the IC50 values for each inhibitor molecule. The results of these experiments (table 2.) have shown that the new, PMO-containing azidated inhibitors were capable of inhibiting the tubulogenesis. Furthermore, in the presence of light, the inhibition was stronger than in the dark, providing evidence for the photoactivation and the concomitant covalent binding of the azidated inhibitors to their target receptors.

The non-halogenated exemplary compounds tested in the tubulogenesis assay showed weaker inhibition, in line with their lower inhibitory potential in the VEGFR2-HEK-based system. Nonetheless the inhibition could clearly be demonstrated, and the effect of the light was straightforward: the illumination potentiated the inhibition by 5-defluoro-5-azido-sunitinib 3.72-fold (Table 2) and the inhibition by 5-defluoro-6-azido-sunitinib 2.52-fold (Table 2). Similarly to the tests performed with transgenic HEK cells, the halogenated versions of the molecules showed stronger baseline inhibition, as evidenced by the stronger decrease of the number of meshes (closed loops) in the network. The effect of the light was similar for these molecules: the inhibition by 6-azido-sunitinib increased 2.62-fold (Table 2) while the inhibition by 5-defluoro-5-azido-6-chloro-sunitinib increased 3.48-fold (Table 2) upon illumination. Finally the inventors could demonstrate that the azidated version of a different scaffold, namely vorolanib, could also inhibit tubulogenesis strongly in the dark (IC50=268 nM, Table 2), and this effect was further potentiated by light (IC50=135 nM, Table 2). For all of these experiments, 5-defluoro-5-azido-6-chloro-vorolanib was used, and 500 nM of Ko-143 (Sigma Aldrich, K2144) was added to the medium since it is known in the art that the efflux transporter ABCG2 is strongly expressed on microvascular endothelial cells and expels vorolanib from the intracellular space. Ko-143 was applied in all conditions (ie. with and without light), and the authors have proven in control experiments that Ko-143 itself has no effect on tubulogenesis. Ko-143 was not added for testing any azidated molecule based on the sunitinib scaffold.

TABLE 2
IC50 values measured on HRMEC-cells in tubulogenesis experiments

| | Figure number | | | |
|---|---|---|---|---|
| Inhibitor (number of formula) | (IC50, network) | Dark | Light | Dark/Light |
| 5-defluoro-5-azido-sunitinib (1) | 19, 24 | 3541 nM | 951 nM | 3.72 |
| 5-defluoro-6-azido-sunitinib (2) | 20, 25 | 1092 nM | 432 nM | 2.52 |
| 6-azido-sunitinib (3) | 21, 26 | 228 nM | 87 nM | 2.62 |
| 5-defluoro-5-azido-6-chloro-sunitinib (5) | 22, 27 | 655 nM | 168 nM | 3.48 |
| 5-defluoro-5-azido-6-chloro-vorolanib (8) | 23, 28 | 686 nM | 173 nM | 3.97 |

### Materials and Methods

To test the effect of light on the inhibitory activity that these compounds exert on VEGF signaling in biological conditions we used a commercially available (BPS BioScience Inc., Cat. No.: 79387) VEGFR2/NFAT Reporter—HEK293 recombinant cell line that increases its luciferase production upon an increase in VEGF signaling. Thus adding exogenous VEGF to the cell culture increases the luminescence produced by the cells while inhibiting VEGFR2 (the receptor of VEGF molecule) results in a decrease in luminescence allowing measurement of inhibitory effect. We have cultured the cells and conducted the measurements according to the manufacturer's instructions.

On the day preceding the measurement, 40 000 recombinant HEK cells were seeded into each well of a 96 well plate in a way that the experiments could be conducted in triplicates. On the day of the measurement, the growth medium (BPS BioScience Inc., Cat. No.: 79528) was replaced with assay medium (BPS BioScience Inc., Cat. No.: 60187-1) and then the cells were pre-incubated with the appropriate inhibitors for one hour (sunitinib, 5-azido-sunitinib, 6-azido-sunitinib). For the conditions requiring irradiation, the plate was placed under a cold white LED light source within the incubator (37° C., 5% CO₂) for the first 10 minutes of the 1-hour-long incubation with the inhibitors to imitate day-light, while the other plate was kept in the dark (also at 37° C., 5% CO₂). Following the pre-incubation with inhibitors we treated the cells for 4 hours with VEGF without removing the inhibitors. For this purpose we used 20 ng/ml of commercially available human VEGF165 (Sf9 derived, from BPS BioScience Inc., Cat. No.: 91001-1) added directly into the cell-culture medium. All the experiments presented in the figures below included a pre-incubation with the appropriate inhibitors (sunitinib, or the indicated azidated molecules containing the PMO) and a subsequent main incubation of 4 hours with VEGF together with the same inhibitors.

After completion of all the incubation steps, the luciferase activity was measured using the commercially available “ONE-step luciferase assay system” (BPS BioScience Inc., Cat. No.: 60690-2) and a CLARIOstar (BMG Labtech) plate reader that quantified luminescence originating from each well of the 96 well plate. The IC50 values were calculated using the GraphPad Prism software.

To test the inhibitory effect of the compounds with light and without light on tubulogenesis [Staton et al., 2009], we have used human retinal microvascular endothelial cells (HRMEC) from Cell Systems (Kirkland, WA 98033, cat. N^{o}: ACBRI 181). VEGF165 (Invitrogen) was only used as a positive control in these experiments, otherwise tubulogenesis relied on endogenous VEGF produced by the culture.

Assays were performed in a 96-well cell culture plate (Biologix). The basement membrane matrix Geltrex (Invitrogen) was thawed on ice overnight before use. 50 μl Geltrex was added to each well with a chilled pipette, then the plate was centrifuged at 2000 rpm for 10 min at 4° C. Subsequently the matrix was let solidify in a humidified incubator at 37° C. for at least 30 minutes. Before seeding, HRMEC cells were treated with compounds (0.1 nM-10 μM) in EBM-2 medium (Lonza) in the dark. After 5 minutes, 100 μl of suspension of treated cells was seeded into each Geltrex-coated well of the plate at a density of 10³ cells/well and then exposed to light treatment (cold white LED light) for 10 min at 37° C. and 5% CO₂ or kept in the dark (control plate) in the incubator during light treatment. Subsequently both the irradiated and the control plates were left in the incubator for 12 hours so that the tubulogenesis can proceed. Thereafter cells were stained with Calcein AM at a concentration of 1.6 μM. Next, the plates were imaged using a Nikon Ti2 inverted microscope applying 4× and 10× objectives and a FITC filter set for Calcein AM. All images were analyzed using the NIS Elements (Nikon) software. A series of images were acquired spanning 120 μm range along the z axis and the built-in algorithm called Extended Depth of Field (EDF) projected structures to create one two-dimensional all-in-focus image. All EDF images were analyzed using the freely customizable NIS-Elements General Analysis 3 module. We set up the experiment routine to segment and identify tubular and nodular structures and extract total and individual tube lengths, tube number, node number and the number of meshes. In all the figures shown in the present patent, the number of meshes is indicated and the change of this particular parameter is shown upon changing the concentration of the inhibitor molecules. IC50 values for tubulogenesis experiments were calculated using such plots and the GraphPad Prism software.

### INDUSTRIAL APPLICATION

The pharmaceutical industry is the major field that can exploit the invention presented in this patent. Azidated molecules can be taken per os and can be targeted to the retina by natural photoactivation for any therapeutic purpose.

The present invention has embodiments that rely on blocking the signal transduction via the VEGFR2 receptor and can be targeted to the retina by the ambient light seen by the patient to be treated. Inhibition of VEGF signaling is currently the main treatment of DR. At present there are close to 500 million diabetic patients worldwide and their number do not stop increasing [Mansour et al., 2020]. Given its much earlier onset, as compared to AMD, DR is the most frequent reason why working adults show visual impairments in developed nations [Heng et al., 2012]. Out of the 93 million people diagnosed globally with DR, 17 million are in the proliferative stage where retinal neovascularization occurs, 21 million suffer from diabetic macular oedema, and 28 million have DR that threatens their eyesight [Heng et al., 2012].

Nonetheless, AMD is a more frequent cause of blindness in the elderly population than DR, and currently used treatments remedying AMD patients also rely on the inhibition of VEGF signaling. This points to a further prominent industrial application of the prototype molecules of the present invention.

Further therapeutic or diagnostic molecules acting on further ocular receptors or enzymes can be developed by capitalizing on the present invention and using already known retinal target molecules, or even retinal target molecules that are to be discovered later. Therefore further diseases of the eye (such as glaucoma) may potentially be cured by exploiting the natural, azidation-based ocular targeting of photoprotected active product ingredients of orally taken medications or eyedrops. The extent of industrial applications and the foreseeable financial benefit of such further embodiments of the present invention will be assessable once the further target biomolecules will be defined in the eye.

### REFERENCES

Andersen, Jacob; Ulf Madsenb; Fredrik Björklinga; Xifu Liang: “Rapid Synthesis of Aryl Azides from Aryl Halides under Mild Conditions” Synlett 2005(14): 2209-2213 DOI: 10.1055/s-2005-872248
Buchy E., et al. Synthesis and Cytotoxic Activity of Self-Assembling Squalene Conjugates of 3-[(Pyrrol-2-yl)methylidene]-2,3-dihydro-1H-indol-2-one Anticancer Agents, European Journal of Organic Chemistry, Volume 2015, Issue 1, 2015, Pages 202-212, https://doi.org/10.1002/ejoc.201403088.
Carpino, Louis A: “1-Hydroxy-7-azabenzotriazole. An efficient peptide coupling additive”. Journal of the American Chemical Society, 1993, 115 (10): 4397-4398. doi:10.1021/ja00063a082
Chen-Rei Wan; Leroy Muya; Viral Kansara; Thomas A Ciulla: “Suprachoroidal Delivery of Small Molecules, Nanoparticles, Gene and Cell Therapies for Ocular Diseases” Pharmaceutics 2021 Feb. 22; 13(2):288. doi: 10.3390/pharmaceutics13020288.
Cohen, Michael N.; Denis O'Shaughnessy; Kate Fisher; Jennifer Cerami; Carl C. Awh; Daniel E Salazar; Philip Rosenfeld; Jeffrey S Heier: “APEX: a phase II randomised clinical trial evaluating the safety and preliminary efficacy of oral X-82 to treat exudative age-related macular degeneration” Br J Ophthalmol. 2021 May; 105(5):716-722. doi: 10.1136/bjophthalmol-2020-316511.
D'Anna, Francesca; Salvatore Marullo; Renato Noto: “Ionic Liquids/[bmim][N3] Mixtures: Promising Media for the Synthesis of Aryl Azides by SNAr.” J. Org. Chem. 2008, 73, 6224-6228 https://doi.org/10.1021/jo800676d
Fagan X. J.; Al-Qureshi S.: “Intravitreal injections: a review of the evidence for best practice.” Clin Exp Ophthalmol. 2013 July; 41(5):500-7. doi: 10.1111/ceo.12026.
Farghaly, Thoraya A., Al-Hasani, Wedian A. & Abdulwahab, Hanan Gaber (2021): An updated patent review of VEGFR-2 inhibitors (2017-present), Expert Opinion on Therapeutic Patents, DOI: 10.1080/13543776.2021.1935872
Fahrenholz, F.; Töth, G., Crause, P.; Eggena, P.; Schwartz, I. L.: “[1,6-alpha-aminosuberic acid, 3-(p-azidophenylalanine), 8-arginine]vasopressin: a new photoaffinity label for hydroosmotic hormone receptors. Characterization of the ligand and irreversible stimulation of hydroosmotic water flow in toad bladder by photoaffinity labeling” J Biol Chem. 1983 Dec. 25; 258(24):14861-7.
Grass G. M.; Robinson J. R. “Mechanisms of corneal drug penetration. I: In vivo and in vitro kinetics” J Pharm Sci. 1988 January; 77(1):3-14. doi: 10.1002/jps.2600770103.
Gribanov, Pavel S.; Maxim A. Topchiy; Yulia D. Golenko; Yana I. Lichtenstein; Artur V. Eshtukov; Vladimir E. Terekhov; Andrey F. Asachenkoa; Mikhail S. Nechaev: “An unprecedentedly simple method of synthesis of aryl azides and 3-hydroxytriazenes” Green Chem., 2016, 18, 5984.
Hajipour, Abdol R. and Fatemeh Mohammadsaleh: “Synthesis of aryl azides from aryl halides promoted by Cu2O/tetraethylammonium prolinate” Tetrahedron Letters, Volume 55, Issue 50, 10 Dec. 2014, Pages 6799-6802. https://doi.org/10.1016/j.tetlet.2014.10.045.
Heng, L. Z.; Comyn O.; Peto T.; Tadros, C.; Ng, E.; Sivaprasad, S; Hykin, P. G.: “Diabetic retinopathy: pathogenesis, clinical grading, management and future developments” Diabet Med. 2013 June; 30(6):640-50. doi: 10.1111/dme.12089.
Ioele G, De Luca M, Garofalo A, Ragno G. Photosensitive drugs: a review on their photoprotection by liposomes and cyclodextrins. Drug Deliv. 2017 December; 24(sup1):33-44. doi: 10.1080/10717544.2017.1386733.
Ioele G, Tavano L, Luca M, Muzzalupo R, Mancuso A, Ragno G. Light-sensitive drugs in topical formulations: stability indicating methods and photostabilization strategies. Future Med Chem. 2017 October; 9(15):1795-1808. doi: 10.4155/fmc-2017-0105.
L. Coelho, I. F. Almeida, J. M. Sousa Lobo, J. P. Sousa e Silva, Photostabilization strategies of photosensitive drugs, International Journal of Pharmaceutics, Volume 541, Issues 1-2, 2018, Pages 19-25, https://doi.org/10.1016/j.ijpharm.2018.02.012.
Jackson, Timothy L.; David Boyer; David M. Brown; Nauman Chaudhry; Michael Elman; Chris Liang; Denis O'Shaughnessy; Edward C. Parsons; Sunil Patel; Jason S. Slakter; Philip J. Rosenfeld: “Oral Tyrosine Kinase Inhibitor for Neovascular Age-Related Macular Degeneration: A Phase 1 Dose-Escalation Study” JAMA Ophthalmol. 2017 Jul. 1; 135(7):761-767.doi: 10.1001/jamaophthalmol.2017.1571.
Keana, John F. W. and Sui Xiong Cai: “New reagents for photoaffinity labeling: synthesis and photolysis of functionalized perfluorophenyl azides” J. Org. Chem. 1990, 55, 11, 3640-3647 https://doi.org/10.1021/jo00298a048
Kennedy, D. P.; Kormos, C. M.; Burdette, S. C. Ferribright: A rationally designed fluorescent probe for redox active metals. J. Am. Chem. Soc. 2009, 131, 8578-8586.
Kessel, Line; Jesper Holm Lundeman; Kristina Herbst; Thomas Vestergaard Andersen; Michael Larsen: “Age-related changes in the transmission properties of the human lens and their relevance to circadian entrainment” J Cataract Refract Surg. 2010 February; 36(2):308-12. doi: 10.1016/j.jcrs.2009.08.035.
Khanwelkar, Rahul R., Chen, Grace Shiahuy, Wanga, Hsiao-Chun et al., Synthesis and structure-activity relationship of 6-arylureido-3-pyrrol-2-ylmethylideneindolin-2-one derivatives as potent receptor tyrosine kinase inhibitors. Bioorganic & Medicinal Chemistry 18 (2010) 4674-4686
Khoo, Chloe; Erin Flynn; Preet Sohal; Rheem Al Shabeeb; Baha El Khatib; Marena Patronas: “Submacular Hemorrhage Following Aflibercept Intravitreal Injection: A Report of Two Cases” Cureus. 2022 Jul. 25; 14(7):e27255. doi: 10.7759/cureus.27255. eCollection 2022 July.
Lombardo M.; Serrao S.; Devaney N.; Parravano M.; Lombardo G.: “Adaptive optics technology for high-resolution retinal imaging.” Sensors (Basel). 2012 Dec. 27; 13(1):334-66. doi: 10.3390/s130100334.
Lonza Press Release “Lonza Expands its Capsugel® Capsule Offering to Include Titanium Dioxide-Free White Hard Gelatin Capsules” May 9, 2022, Basel, Switzerland
Millera, Shelli A. and Leadbeater, Nicholas E. “Direct, rapid, solvent-free conversion of unactivated esters to amides using lithium hydroxide as a catalyst” RSC Adv., 2015, 5, 93248-93251
Montalbetti, C. A. G. N. and Falque, V. “Amide bond formation and peptide coupling”, Tetrahedron, 61(2005), pp. 10827-10852
Nazari-Vanani, R. et al., A novel self-nanoemulsifying formulation for sunitinib: Evaluation of anticancer efficacy. Colloids and Surfaces B: Biointerfaces 160 (2017) 65-72
Peng, Fan-Wei, Liu, Da-Ke, Zhang, Qing-Wen, Xu, Yun-Gen & Shi, Lei (2017) VEGFR-2 inhibitors and the therapeutic applications thereof: a patent review (2012-2016), Expert Opinion on Therapeutic Patents, 27:9, 987-1004, DOI: 10.1080/13543776.2017.1344215
Rajagopalan, R.: “Azide derivatives for phototherapy” WO/2011/084571
Seong, Hyo Jin Seong; Young Min Park; Jiwon Kim; Kang Ju Son; Eun Jee Chung: “Incidence of Acute Endophthalmitis after Intravitreal Anti-Vascular Endothelial Growth Factor Injection in Age-Related Macular Degeneration” Korean J Ophthalmol. 2022 Aug. 19. doi: 10.3341/kjo.2022.0088.
Staton, Carolyn A, Reed, Malcolm W R, Brown, Nicola J: “A critical analysis of current in vitro and in vivo angiogenesis assays” Int J Exp Pathol. 2009 June; 90(3): 195-221. Valeur, E. and Bradley, M. Chem. Soc. Rev., 38 (2009), pp. 606-631
Wormald R.; Evans J.; Smeeth L.; Henshaw K.: “Photodynamic therapy for neovascular age-related macular degeneration.” Cochrane Database Syst Rev. 2005 Oct. 19; (4):CD002030. doi: 10.1002/14651858.CD002030.pub2.
Zhong Wang, Qing Li: “Sunitinib prodrug and pharmaceutical composition” EP3252048A4.
Yamada, Norihiro; Timothy W Olsen: “Routes for Drug Delivery to the Retina: Topical, Transscleral, Suprachoroidal and Intravitreal Gas Phase Delivery” Dev Ophthalmol. 2016; 55:71-83. doi: 10.1159/000431193. Epub 2015 Oct. 26.
Yang T-H et al. “Structural optimization and evaluation of novel 2-pyrrolidone-fused (2-oxoindolin-3-ylidene)methylpyrrole derivatives as potential VEGFR-2/PDGFRβ inhibitors” Chemistry Central Journal, 2017, 11:72 (2017A)
Yang T-H et al., “Synthesis and Evaluation of Novel 2-Pyrrolidone-Fused (2-Oxoindolin-3-ylidene)methylpyrrole Derivatives as Potential Multi-Target Tyrosine Kinase Receptor Inhibitors” Molecules 2017, 22, 913 (2017B)
Yingqian Peng; Luosheng Tang; Yedi Zhou: “Subretinal Injection: A Review on the Novel Route of Therapeutic Delivery for Vitreoretinal Diseases” Ophthalmic Res. 2017; 58(4):217-226. doi: 10.1159/000479157. Epub 2017 Sep. 1.

1-9. (canceled)

10. A method of treating a patient in need of ophthalmic treatment, said patient having an ocular disease, said method comprising administering a compound to said patient, said compound having an azide (N3) moiety comprising an azido group,
wherein said compound is an aryl-azide compound, wherein the azido group forms a reactive radical upon illumination by light in the eye via contacting ambient light,
whereby the active agent moiety can be bound to the binding site of the biological target, the azide moiety can be photoactivated,
whereby the compound modulates the said biological target in the eye of the subject,
wherein said compound has general formula (I.1) 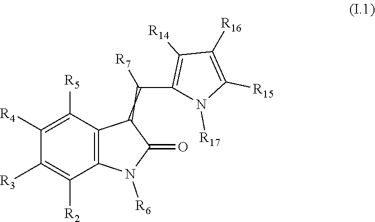
wherein in the formula
at least one of R₂, R₃, R₄ and R₅ is an azido group (N₃);
wherein any one of R₂, R₃, R₄ and R₅ which is different from an azido group, is defined in claim 8,
wherein preferably R₂, R₃, R₄ and R₅, when being different from azido, are, independently, selected from a
H, Me, halogenide, pseudohalogenide, —OH, —SH, —OMe, —OEt, —NO₂, —NH₂, —NHMe, —COOH, CONH₂—CF₃; preferably H, halogenide, pseudohalogenide, —OMe, —OH, —SH,
substituted or unsubstituted C1-C4 alkyl, C1-C4 alkenyl, C1-C4 alkoxy, C1-C4 alkylcarbonyl, C1-C4 alkenylcarbonyl, C6 aryl, C7-C8 alkylaryl (aralkyl), 5 to 6 membered heteroaryl, 6-8 membered alkyl-heteroaryl, said substituent, if any, being selected from halogenide, pseudohalogenide, —OH, —SH, —OMe, —NO₂, —NH₂, —NHMe,
NR₂₁R₂₂, wherein R₂₁ and R₂₂ are, selected from H, methanesulfonyl, ethanesulfonyl, phenylsulfonyl, substituted or unsubstituted C1-C4 alkyl and C1-C4 alkoxy, said substituent, if any, preferably being selected from halogenide, pseudohalogenide, —OH, —SH, —OMe, —NO₂, —NH₂, —NHMe, more preferably halogenide, wherein preferably at least one of R₂₁ and R₂₂ is H, Me or Et,
SO₂NR₂₃R₂₄, wherein R₂₃ and R₂₄ are, independently selected from H, substituted or unsubstituted C1-C4, C6 aryl, C7-C8 alkylaryl (aralkyl), 5 to 6 membered heteroaryl, 6-8 membered alkyl-heteroaryl, said substituent, if any, preferably being selected from halogenide, pseudohalogenide, —OH, —SH, —OMe, —NO₂, —NH₂, —NHMe, more preferably halogenide, wherein preferably at least one of R₂₃ and R₂₄ is H, Me or Et,
ureido, preferably aryl-ureido or heteroaryl-ureido, preferably phenyl-ureido optionally substituted with (preferably in para position) C1-C3 alkyl, C2-C4 alkenyl, C2-C4 alkynyl, C1-C4 alkoxy, halogenide, pseudohalogenide, —OH, —SH, —OMe, —NO₂, —NH₂, —NHMe, in particular C1-C2 alkoxy, halogenide, methyl or unsubstituted, highly preferably (para-metoxy-phenyl)-ureido;
R₆ is as define in claim 8, in particular in i) or in ii),
R₁₄ is selected from H and a C1-C3 alkyl or C2-C3 alkenyl preferably wherein the π electron pair is conjugated with the π electron system of the pyrrole ring, said C1-C3 alkyl or C2-C3 alkenyl being optionally substituted with a group selected from a halo, a C6-C10 aryl or a 5-10 membered heteroaryl, R₁₄ is preferably selected from H and methyl,
R₁₅ is selected from H and a C1-C3 alkyl or C2-C3 alkenyl preferably wherein the π electron pair is conjugated with the π electron system of the pyrrole ring, said C1-C3 alkyl or C2-C3 alkenyl being optionally substituted with a group selected from a halo, a C6-C10 aryl or a 5-10 membered heteroaryl, R₁₅ is preferably selected from H and methyl,
R₁₆ is selected from
H, substituted or unsubstituted C1-C4 alkyl, C2-C4 alkenyl, C2-C4 alkynyl, C1-C4 alkoxy, C1-C4 alkylcarbonyl, C1-C4 alkenylcarbonyl, C1-C4 alkynylcarbonyl, C1-C4 alkylamide, C1-C4 amide, C1-C4 alkylcarboxyl, a C2-C4 alkenylcarboxyl, a C2-C4 alkynylcarboxyl, and a C2-C4 carboxylate ester said substituent, if any, being selected from halogenide, pseudohalogenide, —OH, —SH, —OMe, —NO₂, —NH₂, —NHMe, and
preferably, a substituted amine or amide wherein preferably said amid is bound via the carbonyl to the pyrrole ring thereby the π electrons of the oxo group forming part of the conjugated system,
said amine or amide substituent being selected from
substituent S₁ being a C1-C4 alkyl (preferably C1-C4 or C2-C3 alkyl) preferably substituted with a substituent selected from
an amine; said amine being optionally substituted with one or two C1-C4 or C2-C3 alkyl or a group as defined as substituent S₃ below, optionally a cyclic polyether forming a tertiary amine, (i.e. said amine being a secondary or tertiary amine), and
a group as defined as substituent S₂ below,
substituent S₂ being a 5 to 10 membered (preferably 5 to 6 membered) heterocycle, preferably heteroaryl and a C6-C10 aryl, said heterocycle or aryl being optionally substituted with a group having the formula X—R₁₀ wherein X is selected from NH, NR₁₁, R₁₁ being selected from C1-C3 alkyl, C1-C3 alkenyl and C1-C3 alkoxy), O, S, C1-C3 alkyl and C1-C3 alkenyl, and R₁₀ is selected from a 5 to 10 membered heterocycle or a C6-C10 aryl, optionally further substituted with 1 to 4 membered group selected from alkyl, alkenyl, amide, carboxyl alkylcarbonyl, alkoxy and halogenide,
substituent S₃ being a polyether, preferably a polyethylene glycol, wherein the number ether —O— is 2 to 12, preferably 3 to 9, (or in an alternative embodiment R₁₆ is a group as defined for R₁₅
R₇ is H or a C1-C4 alkyl, a C6-C10 aryl or a 5 to 6 membered heterocycle, preferably heteroaryl, H, amine, amide and C1-C3 alkenyl, a C1-C4 alkylcarbonyl, C1-C4 alkenylcarbonyl, C1-C4 alkynylcarbonyl, a C1-C4 alkylcarboxyl, C1-C4 alkenylcarboxyl, C1-C4 alkynylcarboxyl or C1-C4 carboxylate ester, wherein if any of R₇ and R₈ is substituted, said substituent, if any, being selected from halogenide, pseudohalogenide, —OH, —SH, —OMe, —NO₂, —NH₂, —NHMe,
R17 is selected from H and optionally an in vivo metabolizable group whereby the compound is a prodrug; preferably an intracellularly metabolizable group; preferably R₁₇, once present, is i) a substituted or unsubstituted C1-C4 alkyloxy group linked via a carbon to the nitrogene atom of the oxindole structure (in particular a C1-alcoxy, preferably a CH₂—O— moiety) preferably acylated to be an ester by an —C(O)—R₄, group, wherein R₄₁ is selected from the group consisting of OR₄₂, SR₄₂, and N(R₄₂)₂; and
R₄₁ or R₄₂ is selected from the group consisting of
H, unsubstituted or substituted C1-C30 alkyl, preferably a C1-C12 alkyl, more preferably a C1-C8 or a C1-C6 alkyl, in particular a C1-C4 alkyl, C2-C30 alkenyl preferably a C1-C12 alkenyl, more preferably a C1-C8 or a C1-C4 alkenyl, in particular a C1-C6 alkenyl, C2-C30 alkynyl, preferably a C1-C12 alkynyl, more preferably a C1-C8 or a C1-C6 alkynyl, in particular a C1-C4 alkynyl; C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group; or wherein the said alkyl, alkenyl or alkyl group has a substitutent (is substituted) by said cycloalkyl, aryl, heterocyclyl, heteroaryl, hydroxylalkyl, carboxylalkyl or alkylamido group,
said substituent of the C1-C4 alkyloxy group if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe,
ii) or R₁₇ is a or a C(O)—R₃₃ group forming an amide bond with the ring N, wherein R₃₃ group is selected from C1-C30 alkyl, preferably a C1-C12 alkyl, more preferably a C1-C8 or a C1-C6 alkyl, in particular a C1-C4 alkyl, C2-C30 alkenyl, preferably a C1-C12 alkenyl, more preferably a C1-C8 or a C1-C6 alkenyl, in particular a C1-C4 alkenyl, C2-C30 alkynyl, preferably a C1-C12 alkynyl, more preferably a C1-C8 or a C1-C6 alkynyl, in particular a C1-C4 alkynyl; C3-C8 cycloalkyl, C6-C10 aryl, 4-15 membered heterocyclyl, 5-15 membered heteroaryl, hydroxyl C1-C6 alkyl, carboxyl C1-C6 alkyl, C1-C6 alkyl amido and phosphate group; or wherein the said alkyl, alkenyl or alkyl group has a substitutent (is substituted) by said cycloalkyl, aryl, heterocyclyl, heteroaryl, hydroxylalkyl, carboxylalkyl or alkylamido group, wherein in a particular embodiment said R₃₃ group is selected from a C1-C8 alkyl, in particular a C1-C4 alkyl, wherein the said alkyl, has a substituent (is substituted by a group selected from) a 4-15 membered heterocyclyl,
in a particularly preferred embodiment R₁₇ comprises a biotinyl group with or without a linker,
iii) or in an alternative embodiment R₁₇ is selected from a substituted or unsubstituted C1-C4 alkyl, C2-C4 alkenyl, C2-C4 alkynyl, C2-C8 alkylester, C3-C8 alkenylester or C3-C8 alkynylester, said substituent, if any, being selected from halogenide, pseudohalogenide, —OH, —SH, —OMe, —NO₂, —NH₂, —NHMe,
iv) or R₁₇ is a substituted or unsubstituted C1-C4 alkyloxy group linked via the alkyloxy oxygen to the ring N forming an N—O bond, said substituent on the C1-C4 alkyloxy group (in particular a C1-alcoxy, preferably a CH₂—O— moiety), if any, being selected from the group consisting of H, C1-C6 alkyl, C2-C6 alkenyl, C2-C6 alkynyl, C3-C8 cycloalkyl, C6-C10 aryl, 4-15 membered heterocyclyl and 5-15 membered heteroaryl; as well as halogenide, pseudohalogenide, —OH, —SH, —OMe, —NO₂, —NH₂, and —NHMe;
in particular as defined in i) or in ii).

11. The method for use according claim 10, wherein said compound has general formula (II) 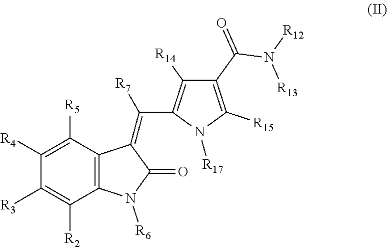
wherein at least one of R₂, R₃, R₄ and R₅ is an azido group (N₃);
wherein any of R₂, R₃, R₄ and R₅ which is different from an azido group, is selected from the group as defined in claim 8, preferably as defined in claim 10, or more preferably is selected from
H, Me, halogenide, pseudohalogenide, —OH, —SH, —OMe, —OEt, —NO₂, —NH₂, —NHMe, —COOH, CONH₂—CF₃; preferably H, halogenide, pseudohalogenide, —OMe, —OH, —SH, preferably —H, halogenide, pseudohalogenide, —OMe, —OH, —SH, in particular H or halogenide,
substituted or unsubstituted C1-C4 alkyl, C1-C4 alkenyl, C1-C4 alkoxy, C1-C4 alkylcarbonyl, C1-C4 alkenylcarbonyl, C6 aryl, C7-C8 alkylaryl (aralkyl), 5 to 6 membered heteroaryl, 6-8 membered alkyl-heteroaryl, said substituent, if any, being selected from halogenide, pseudohalogenide, —OH, —SH, —OMe, —NO₂, —NH₂, —NHMe, preferably H and halogenide, —NR₂₁R₂₂, wherein R₂₁ and R₂₂ are, selected from H, methanesulfonyl, ethanesulfonyl, phenylsulfonyl, substituted or unsubstituted C1-C3 alkyl and C1-C3 alkoxy, said substituent, if any, preferably being selected from halogenide, pseudohalogenide, —OH, —SH, —OMe, —NO₂, —NH₂, —NHMe, preferably —OH, —OMe, —NH₂, —NHMe and halogenide, more preferably halogenide, wherein preferably at least one of R₂₁ and R₂₂ is H, Me or Et, preferably H or Me,
SO₂NR₂₃R₂₄, wherein R₂₃ and R₂₄ are, independently selected from H, substituted or unsubstituted C1-C4, C6 aryl, C7-C8 alkylaryl (aralkyl), 5 to 6 membered heteroaryl, 6-8 membered alkyl-heteroaryl, said substituent, if any, preferably being selected from halogenide, pseudohalogenide,
OH, —SH, —OMe, —NO₂, —NH₂, —NHMe, more preferably halogenide, wherein preferably at least one of R₂₃ and R₂₄ is H, Me or Et,
phenyl-ureido optionally substituted in para position with C1-C3 alkyl, C2-C4 alkenyl, C2-C4 alkynyl, C1-C4 alkoxy, halogenide, pseudohalogenide, —OH, —SH, —OMe, —NO2, —NH2, —NHMe, in particular C1-C2 alkoxy, halogenide, methyl or unsubstituted, highly preferably (para-metoxy-phenyl)-ureido;
wherein R₆ and R₁₇ are as defined above, preferably R₆ being a metabolizable group and preferably R₁₇ being H,
wherein R₁₄, R₁₅ and R₇ is as defined in claim 8, preferably as defined claim 10, more preferably
R₁₂ and R₁₃ are independently selected from H, C1-C8 alkyl, preferably C1-C4 or C2-C3 alkyl, preferably substituted with a substituent selected from one or two C1-C4 or C2-C3 alkyl or a polyether, preferably a polyethylene glycol, wherein the number ether —O— is 2 to 12, preferably 3 to 9, optionally a cyclic polyether forming a tertiary amine, wherein optionally R₁₅ and R₁₃ or R₁₄, together with the backbone atoms, form a 5 to 8 membered heterocycle, preferably a 5 or 6 to 7 membered, preferably a 5 or 6 membered, in particular a 6 membered heterocycle; or amine; said amine being optionally substituted with one or two C1-C4 or C2-C3 alkyl or a group as defined as substituent S₃ below, optionally a cyclic polyether forming a tertiary amine, (i.e. said amine being a secondary or tertiary amine).

12. The method for use according to claim 11 for use in the treatment of an ocular disease that involves a targetable endogenous biomolecule.

13. The method for use according to claim 11 for use in the treatment of an ocular disease selected from the group consisting of
macular degeneration, in particular age-related macular degeneration (AMD),
retinopathies, in particular diabetic retinopathies, proliferative retinopathies, e.g proliferative diabetic retinopathy (PDR),
macular oedema, in particular diabetic macular oedema (DME),
retinal vein occlusion (RVO),
open angle glaucoma (OAG),
angle closure glaucoma (ACG),
congenital glaucoma (CoG), and
any other disease where ocular neovascularization is part of the pathomechanism.

14. The method for use according to claim 11, said compound having general formula selected from the group consisting of general formulae (V.1), (VI.1), (VII.1) and (VIII.1): 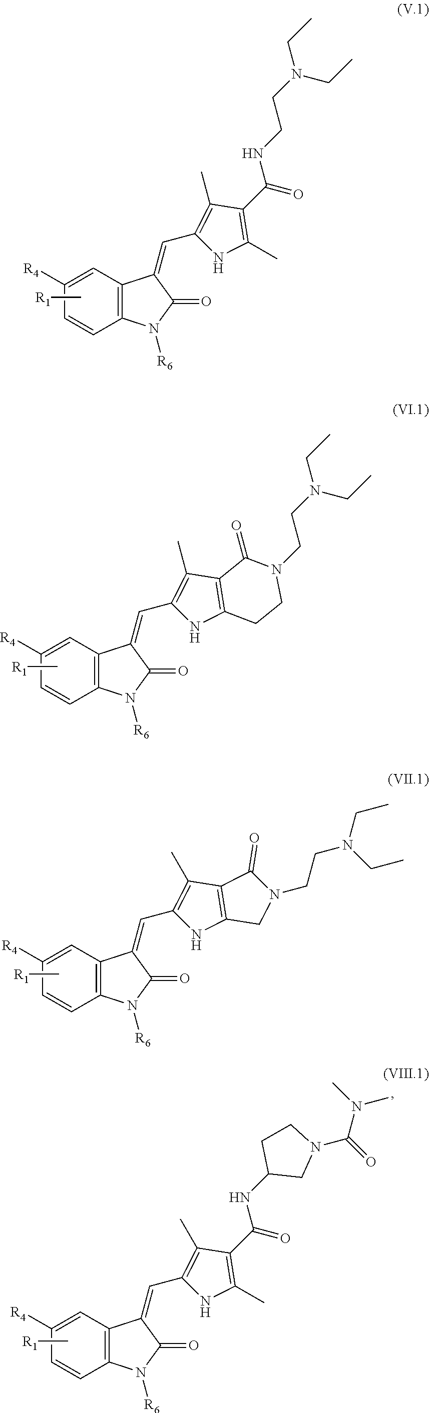
or said compound having general formula selected from the group consisting of general formulae (V), (VI), (VII) and (VIII): 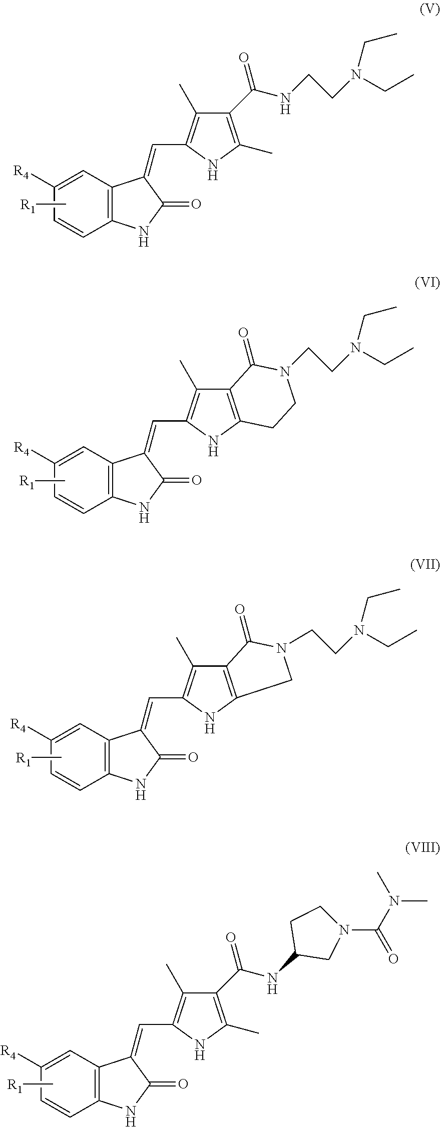
wherein R₁ is an azido group connected to carbon 4, 6 or 7 of the indole-2-one moiety, and
R₄ is selected from the group consisting of H, Me, halogenide, pseudohalogenide, —OH, —SH, —OMe, —OEt, —NO₂, —NH₂, —NHMe, —COOH, CONH₂—CF₃; preferably H, halogenide, pseudohalogenide, —OMe, —OH, —SH, in particular halogenide, highly preferably F or Cl
wherein R₆ group on the nitrogen atom of the indoline-2-one moiety, if present, is as defined in claim 8, 10 or 11.

15. The method for use according to claim 11, said compound having general formula selected from the group consisting of general formulae (V.1), (VI.1), (VII.1) (VIII.1) (V), (VI), (VII) and (VIII):
wherein R₁ is connected to carbon 4, 6 or 7 of the indole-2-one moiety and is selected from the group consisting of H, Me, halogenide, pseudohalogenide, —OH, —SH, —OMe, —NO₂, —NH₂, —NHMe, in particular H and halogenide, highly preferably F or Cl and
R₄ is an azido group,
wherein R₆ group on the nitrogen atom of the indoline-2-one moiety, if present, is as defined in claim 8, 10 or 11.

16. The method for use according to claim 15, and said compound being selected from the following compounds:

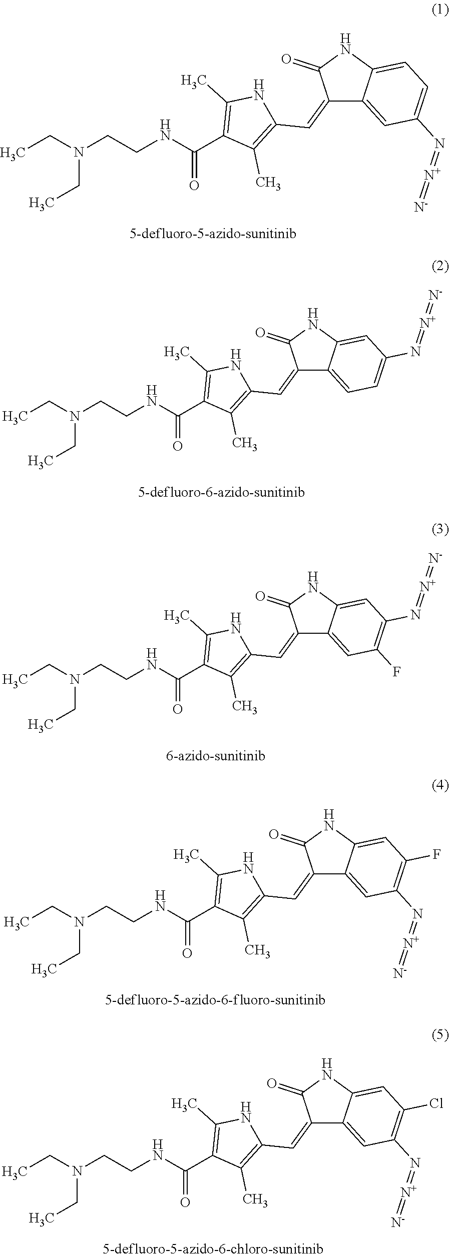 [SMILES: CCN(CC)CCNC(=O)c1c(C)[nH]c(/C=C2\C(=O)Nc3ccc(N=[N+]=[N-])cc32)c1C ; CCN(CC)CCNC(=O)c1c(C)[nH]c(/C=C2\C(=O)Nc3cc(N=[N+]=[N-])ccc32)c1C ; CCN(CC)CCNC(=O)c1c(C)[nH]c(/C=C2\C(=O)Nc3cc(N=[N+]=[N-])c(F)cc32)c1C ; CCN(CC)CCNC(=O)c1c(C)[nH]c(/C=C2\C(=O)Nc3cc(F)c(N=[N+]=[N-])cc32)c1C ; CCN(CC)CCNC(=O)c1c(C)[nH]c(/C=C2\C(=O)Nc3cc(Cl)c(N=[N+]=[N-])cc32)c1C]

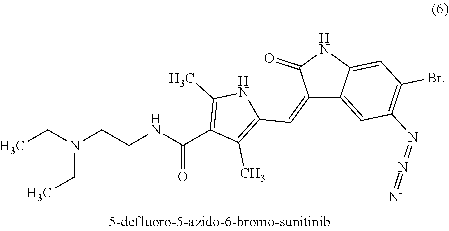 [SMILES: CCN(CC)CCNC(=O)c1c(C)[nH]c(/C=C2\C(=O)Nc3cc(Br)c(N=[N+]=[N-])cc32)c1C]

17. The method for use according to claim 15, said compound being selected from the following compounds.

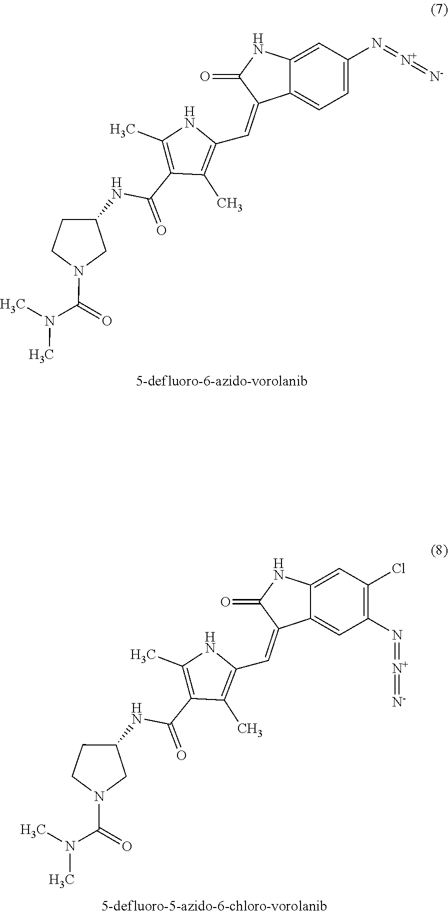 [SMILES: Cc1[nH]c(/C=C2\C(=O)Nc3cc(N=[N+]=[N-])ccc32)c(C)c1C(=O)N[C@H]1CCN(C(=O)N(C)C)C1 ; Cc1[nH]c(/C=C2\C(=O)Nc3cc(Cl)c(N=[N+]=[N-])cc32)c(C)c1C(=O)N[C@H]1CCN(C(=O)N(C)C)C1]

18. The method for use according to claim 10, said compound binding to the biological target, said biological target being a receptor or an enzyme, in an assay.

19. The method for use according to claim 18, said compound inhibiting VEGFR2 in a VEGFR2 inhibition assay, preferably in vitro.

20. A pharmaceutical composition for ophthalmic use, said pharmaceutical composition comprising a compound as defined in claim 10, and a pharmaceutically acceptable excipient.

21. The pharmaceutical composition according to claim 20, which is an oral pharmaceutical composition for ophthalmic use, wherein said composition is formulated to protect the compound from light and/or to prevent light-induced excitation, unwanted photoactivation or decomposition of the azido group.

22. The pharmaceutical composition according to claim 20, which is an eye-drop formulation for ophthalmic use, wherein said composition is formulated to protect the compound from light and/or to prevent light-induced excitation, unwanted photoactivation or decomposition of the azido group.

23. A compound having general formula (I.1) 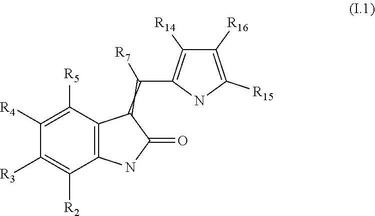
wherein in the formula R₂, R₃, R₄, R₅ R₇, R₁₄, R₁₅ and R₁₆ are as defined in claim 10,
preferably, where appropriate, as defined in claim 11.

24. The compound according to claim 23, wherein said compound has general formula (II) 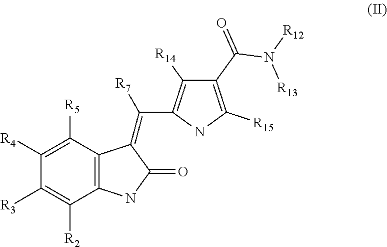
wherein R₂, R₃, R₄, R₅ R₇, R₁₂, R₁₃, R₁₄, R₁₅ and R₁₆ are as defined in claim 11.

25. The compound according to claim 24, said compound having general formula selected from the group consisting of general formulae (V.1), (VI.1), (VII.1) and (VIII.1): 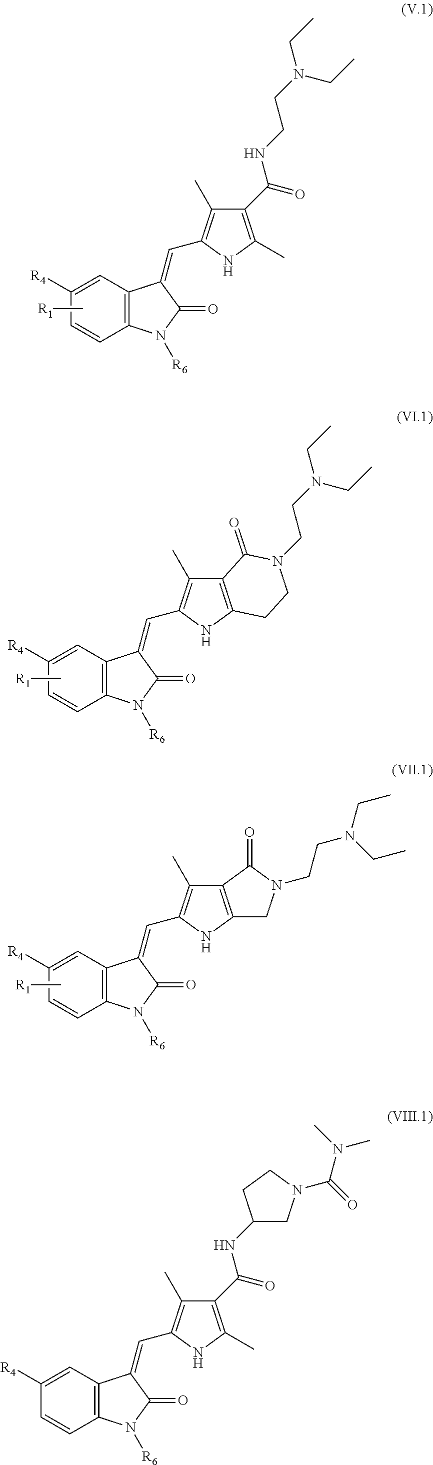
or
said compound having general formula selected from the group consisting of general formulae (V), (VI), (VII) and (VIII): 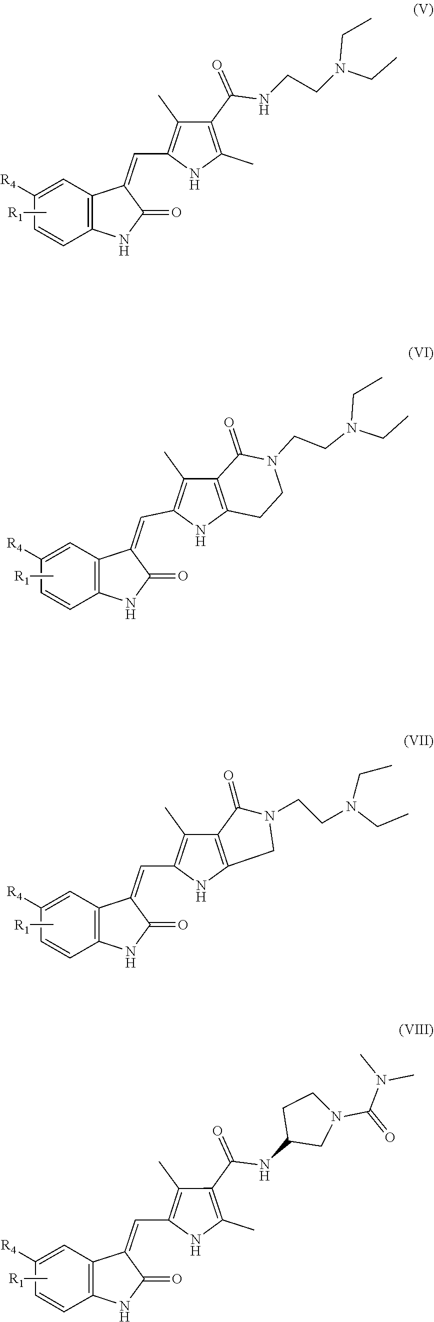
wherein R₁ is an azido group connected to carbon 4, 6 or 7 of the indole-2-one moiety, and
R₄ is selected from the group consisting of H, Me, halogenide, pseudohalogenide, —OH, —SH, —OMe, —OEt, —NO₂, —NH₂, —NHMe, —COOH, CONH₂—CF₃; preferably H, halogenide, pseudohalogenide, —OMe, —OH, —SH, in particular halogenide, highly preferably F or Cl, wherein R₆ group on the nitrogen atom of the indoline-2-one moiety, if present, is as defined in claim 8.

26. The compound according to claim 24, said compound having general formula selected from the group consisting of general formulae (V.1), (VI.1), (VII.1) (VIII.1) (V), (VI), (VII) and (VIII):
wherein R₁ is connected to carbon 4, 6 or 7 of the indole-2-one moiety and is selected from the group consisting of H, Me, halogenide, pseudohalogenide, —OH, —SH, —OMe, —NO₂, —NH₂, —NHMe, in particular H and halogenide, highly preferably F or Cl and
R₄ is an azido group,
wherein R₆ group on the nitrogen atom of the indoline-2-one moiety, if present, is as defined in claim 8.

27. The compound according to claim 24, said compound being selected from the following compounds:

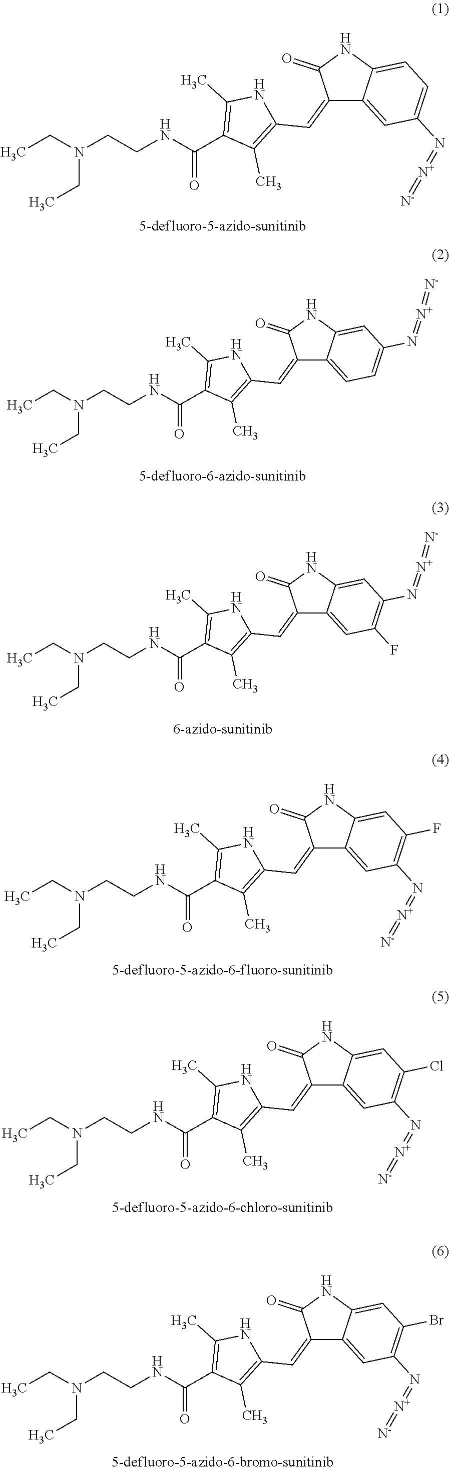 [SMILES: CCN(CC)CCNC(=O)c1c(C)[nH]c(/C=C2\C(=O)Nc3ccc(N=[N+]=[N-])cc32)c1C ; CCN(CC)CCNC(=O)c1c(C)[nH]c(/C=C2\C(=O)Nc3cc(N=[N+]=[N-])ccc32)c1C ; CCN(CC)CCNC(=O)c1c(C)[nH]c(/C=C2\C(=O)Nc3cc(N=[N+]=[N-])c(F)cc32)c1C ; CCN(CC)CCNC(=O)c1c(C)[nH]c(/C=C2\C(=O)Nc3cc(F)c(N=[N+]=[N-])cc32)c1C ; CCN(CC)CCNC(=O)c1c(C)[nH]c(/C=C2\C(=O)Nc3cc(Cl)c(N=[N+]=[N-])cc32)c1C ; CCN(CC)CCNC(=O)c1c(C)[nH]c(/C=C2\C(=O)Nc3cc(Br)c(N=[N+]=[N-])cc32)c1C]

28. The compound according to claim 24, said compound being selected from the following compounds:

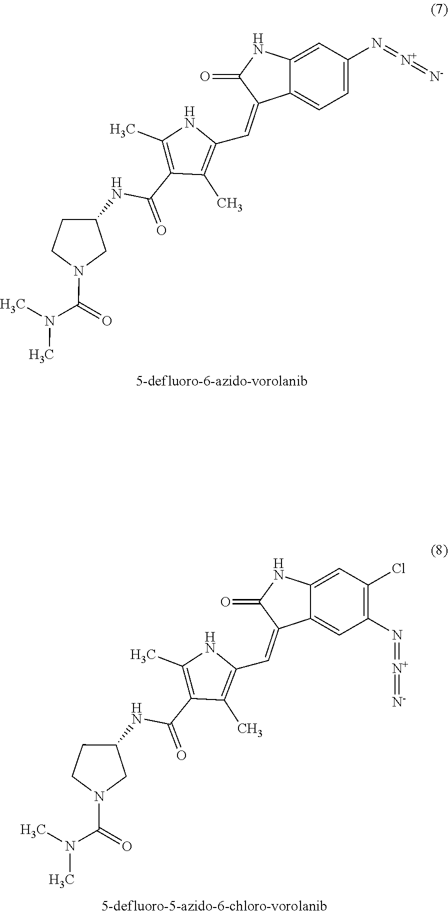 [SMILES: Cc1[nH]c(/C=C2\C(=O)Nc3cc(N=[N+]=[N-])ccc32)c(C)c1C(=O)N[C@H]1CCN(C(=O)N(C)C)C1 ; Cc1[nH]c(/C=C2\C(=O)Nc3cc(Cl)c(N=[N+]=[N-])cc32)c(C)c1C(=O)N[C@H]1CCN(C(=O)N(C)C)C1]

29. (canceled)

30. The method according to claim 10 wherein said compound is a VEGF or PDGF signaling inhibitor, preferably a VEGF-receptor inhibitor, in particular a VEGFR inhibitor selected from the group consisting of VEGFR1, VEGFR2 and VEGFR3 inhibitors, preferentially a VEGFR2 inhibitor.
